(19) United States
(12) Patent Application Publication
(10) Pub. No.: US 2017/0273308 A1
(43) Pub. Date: Sep. 28, 2017

(54) NOVEL BACTERIAL ENDOPHYTE WITH ANTIFUNGAL ACTIVITY

(71) Applicant: University of Guelph, Guelph (CA)

(72) Inventors: Manish N. Raizada, Milton (CA); Walaa Mousa, Guelph (CA); Charles Schearer, Guelph (CA)

(73) Assignee: University of Guelph, Guelph (CA)

(21) Appl. No.: 15/514,241

(22) PCT Filed: Sep. 28, 2015
(86) PCT No.: PCT/CA2015/050972

§ 371 Date: Mar. 24, 2017

Related U.S. Application Data
(60) Provisional application No. 62/056,012, filed on Sep. 26, 2014.

(51) Int. Cl. A01N 63/00; C07K 14/265; C12N 9/16; C12N 9/42; C12N 9/12; C12N 9/02; C12N 15/82; C12N 9/00

(52) U.S. Cl. CPC A01N 63/00; C12N 15/8282; C07K 14/265; C12N 9/93; C12N 9/2442; C12N 9/1241; C12N 9/0071; C12N 9/16; C12Y 602/01003; C12Y 207/07065; C12Y 302/01014; C12Y 301/04; C12Y 114/14009

(57) ABSTRACT

An Enterobacter species isolated from finger millet, characterized by 16S rRNA gene analysis and the identification of genes that prevent or inhibit the growth of fungal plant pathogens, is disclosed for use with agricultural plants.

### RELATED APPLICATIONS

This application claims the benefit of priority of U.S. Provisional Patent Application Ser. No. 62/056,012 filed on Sep. 26, 2014 which is hereby incorporated by reference in its entirety.

### INCORPORATION OF SEQUENCE LISTING

A computer readable form of the Sequence Listing “6580-P47034PC00_SequenceListing.txt” (87.1 KB), submitted by ePCT and created on Sep. 28, 2015, is herein incorporated by reference.

### FIELD

The present disclosure relates to a novel bacterial endophyte with antifungal activity isolated from finger millet.

### BACKGROUND

Finger millet [Eleusine coracana (L.) Gaertn.] is a crop that tolerates stress conditions and that resists diverse pathogens [1]. Though limited scientific research has been conducted on this crop, comparative analysis data from tropical Africa (Burundi) showed that whereas 92-94 identifiable fungal mould species were found in maize grain and 97-99 in sorghum, only 4 were found in finger millet grain [3]. Furthermore, whereas 295-327 non-identified mould colonies were found in maize grain, and 508-512 in sorghum, only 4 were found in finger millet grain [3].

Fusarium is a widespread pathogen of cereal crops, including F. verticillioides in tropical maize which is associated with the production of carcinogenic mycotoxins, and F. graminearum, the causal agents of Gibberella ear rot in maize and Fusarium head blight in wheat; the latter diseases are associated with the mycotoxin deoxynivalenol (DON) [4]. Multiple studies have reported the presence of Fusarium in finger millet in India [5,6] and Africa [7], including F. graminearum in India [8,9] and Africa [5,10]. However, despite its prevalence as a disease-causing agent across cereals, Fusarium is not considered to be an important pathogen of finger millet, suggesting this crop has tolerance to this family of pathogenic fungi. Moreover, in the above African study [3], no Fusarium species were identified in finger millet grain, compared to 28-40 Fusarium species in maize grain, 11-25 in sorghum, 25-51 in common bean, 4-16 in peanut and 29-43 in mung bean.

The resistance of finger millet grain to mould has been attributed to abundant polyphenols [11,12]. However, an emerging body of literature suggests that microbes that reside in plants without themselves causing disease, defined as endophytes, may contribute to host resistance against fungal pathogens [13,14]. The mechanisms involved in endophyte-mediated disease resistance include competition for nutrients and space [15], induction of host resistance genes [16], improvement of host nutrient status [17], and/or production of anti-pathogenic natural compounds [14]. It was hypothesized that endophytes might contribute to the resistance of finger millet to Fusarium reported by local farmers.

Fusarium are ancient fungal species, dated to at least 8.8 millions of years ago, and their diversification appears to have co-occurred with that of the C4 grasses (which includes finger millet), certainly pre-dating finger millet domestication in Africa [18]. A diversity of F. verticillioides (synonym F. moniliforme) has been observed in finger millet in Africa and it has been suggested that the species evolved there [5]. These observations suggest the possibility of co-evolution within finger millet between Fusarium and competitive fungal endophytes. Reports of endophytes isolated from finger millet have not been found.

### SUMMARY

The inventors isolated a bacterial endophyte from finger millet with anti-Fusarium activity. Sequencing the 16S rRNA gene of the isolated bacterial endophyte revealed that it is a novel Enterobacter species.

Accordingly, one aspect of the disclosure provides an isolated bacteria having a 16S rRNA gene comprising a nucleotide sequence that has at least 96% sequence identity to the sequence set forth in SEQ ID NO:1, or its progeny, or mutants thereof. In one embodiment, the bacteria is a bacterial endophyte.

In one embodiment, the bacteria is an Enterobacter species.

In another embodiment, the bacteria inhibits the growth of at least one fungal pathogen.

In one embodiment, fungal pathogen belongs to class Eurotiomycetes or to class Sordariomycetes. In one embodiment, the fungal pathogen belongs to order Eurotiales, Hypocreales, or Trichosphaeriales. In one embodiment, the fungal pathogen belongs to family Nectriaceae, Trichocomaceae or Hypocreaceae

In another embodiment, the fungal pathogen is Fusarium, optionally, Fusarium graminearum. In one embodiment, the fungal pathogen is Aspergillus.

In another embodiment, the fungal pathogen is selected from the group consisting of Aspergillus flavus, Aspergillus niger, Fusarium lateritium, Fusarium avenaceum, Fusarium sporotrichoides, Fusarium graminearum, Paraconiothyrium brasiliense, Penicillium expansum Nigrospora oryzae and Trichoderma hamatum.

In one embodiment, the bacteria comprises at least one gene that is induced as a result of contact with Fusarium mycelium. In one embodiment, the gene has at least 80% sequence identity with a nucleic acid sequence selected from any one of SEQ ID NOs: 2-11 or comprises a nucleic acid sequence selected from any one of SEQ ID NOs: 2-11.

In another aspect, the disclosure provides a composition comprising the bacteria described herein, and optionally a carrier.

In one embodiment, the composition is in a fluid form suitable for spray application or for coating seeds.

In another aspect, the disclosure provides a synthetic combination of the bacteria as described herein in association with a plant. In one embodiment, the bacteria resides within the seeds, roots, stems and/or leaves of the plant as an endophyte. In one embodiment, the bacterial endophyte are heterologous to the microbial population of the plant. In one embodiment, the plant is maize, wheat, sorghum or barley.

In one embodiment, there is provided a synthetic combination comprising a purified bacterial population in association with a plurality of seeds or seedlings of an agricultural plant, wherein the purified bacterial population comprises an endophyte that is heterologous to the seeds or seedlings and comprises a 16S rRNA nucleic acid sequence at least 96% sequence identical to the sequence set forth in SEQ ID NO:1 as described herein. In one embodiment, the endophyte is present in the synthetic combination in an amount effective to provide a benefit to the seeds or seedlings or plants derived from the seeds or seedlings.

In one embodiment, the benefit is selected from the group consisting of decreased ear rot, decreased kernel rot, decreased head blight, improved growth, increased mass, increased grain yield, and decreased levels of deoxynivalenol.

In one embodiment, the agricultural plant is a cereal, optionally maize, wheat, sorghum or barley.

In another aspect, the disclosure provides a method of preventing or inhibiting fungal growth on a plant, comprising inoculating a plant with the bacteria or composition described herein.

In one embodiment, the plant inoculated is an agricultural plant. In one embodiment, the agricultural plant is a monocot. In one embodiment, the agricultural plant belongs to the Poaceae family.

In another embodiment, the agricultural plant is a cereal. Optionally, the cereal is maize, wheat, sorghum or barley.

In another embodiment, inoculating a plant comprises coating the seeds of the plant and/or exposing the plant to a spray.

In another aspect, the disclosure provides a method of preventing or inhibiting fungal growth on a plant, comprising contacting the surface of a plurality of seeds or seedlings with a formulation comprising a purified bacterial population that comprises an endophyte that is heterologous to the seeds or seedlings and comprises a 16S rRNA nucleic acid sequence at least 96% sequence identical to the sequence set forth in SEQ ID NO:1 as described herein. In one embodiment, the endophyte is present in the synthetic combination in an amount effective to prevent or inhibit fungal growth on the plants derived from the seeds or seedlings.

In another aspect, the disclosure provides a method for preparing an agricultural seed composition, comprising contacting the surface of a plurality of seeds with a formulation comprising a purified bacterial population that comprises an endophyte that is heterologous to the seeds and comprises a 16S rRNA nucleic acid sequence at least 96% sequence identical to the sequence set forth in SEQ ID NO:1 as described herein. In one embodiment, the endophyte is present in the synthetic combination in an amount effective to provide a benefit to the seeds or the plants derived from the seeds. In one embodiment, the benefit is selected from the group consisting of decreased ear rot, decreased kernel rot, decreased head blight, improved growth, increased mass, increased grain yield, and decreased levels of deoxynivalenol.

The present inventors also identified a number of genes that are required for the antifungal activity of the isolated endophyte. Accordingly, the disclosure provides an isolated gene, wherein said gene comprises a nucleic acid sequence that has at least 80% sequence identity with a nucleic acid sequence selected from any one of SEQ ID NOs: 2-11, or the complement thereof. In one embodiment, the gene encodes for a protein that has at least 80% sequence identity with the amino acid sequence of any one of SEQ ID NOs: 12-21.

Also provided is a recombinant construct comprising an isolated gene that has at least 80% sequence identity with a nucleic acid sequence selected from any one of SEQ ID NOs: 2-11, or the complement thereof. In one embodiment, the gene is operably linked to a promoter and/or other regulatory sequence.

A further aspect of the disclosure is a transformed bacterial cell, plant cell, plant or plant part expressing a nucleic acid molecule comprising a nucleic acid sequence that has at least 80% sequence identity with a nucleic acid sequence selected from any one of SEQ ID NOs: 2-11, or the complement thereof. In one embodiment, the bacterial cell, plant cell, plant or plant part is resistant to fungal infection. In another embodiment, there is provided a transformed bacterial cell, plant cell, plant or plant part expressing a protein that has at least 80% sequence identity with any one of SEQ ID NOs: 12-21, wherein said bacterial cell, plant cell, plant or plant part is resistant to fungal infection. In one embodiment, the transformed bacterial cell, plant cell, plant or plant part is resistant to fungal infection by Fusarium, optionally F. graminearum.

Yet another aspect of the disclosure provides a method of increasing the resistance of a bacterial cell, plant cell, plant or plant part to a fungal pathogen comprising transforming the bacterial cell, plant cell, plant or plant part with an isolated gene or recombinant construct as described herein and expressing the transformed gene or nucleic acid in the bacterial cell, plant cell, plant or plant part. In one embodiment, the gene comprises a nucleic acid sequence that has at least 80% sequence identity with a nucleic acid sequence selected from any one of SEQ ID NOs: 2-11, or the complement thereof. In one embodiment, the fungal pathogen is Fusarium, optionally F. graminearum.

In another aspect, the disclosure provides a method for reducing the level of deoxynivalenol (DON) in a plant during storage, comprising inoculating the plant with a bacteria or composition as described herein. In one embodiment, the levels of DON in the plant are reduced relative to a plant that has not been inoculated with the bacterial endophyte or composition as described herein.

In another aspect, there is provided a synthetic combination of a bacteria as described herein in association with a plant. In one embodiment, the bacteria resides within the seeds, roots, stems and/or leaves of the plant as an endophyte. In one embodiment, the bacteria comprises a 16S rRNA nucleic acid sequence with at least 96% sequence identical to the sequence set forth in SEQ ID NO:1. In one embodiment, the plant is a seed or seedling. In one embodiment, the bacteria is heterologous to the microbial population of the plant. In one embodiment, the plant is an agricultural plant other than millet. In one embodiment, the agricultural plant is a cereal, optionally maize, wheat, sorghum or barley.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTIONS OF DRAWINGS

FIG. 1 depicts finger millet and anti-Fusarium bacterial endophytes isolated in this study. (A) depicts a picture of finger millet plant, (B) depicts a plate showing diverse endophytes in the root extract, (C) depicts a picture of the candidate endophyte, M6 and (D) depicts a plate showing the in vitro agar diffusion anti-Fusarium assay. (E) depicts quantification of the inhibitory effect of the endophytes or fungicide control (amphotericin B at concentrations of 5 μg/ml), on the growth of F. graminearum in vitro. For these experiments, n=3. The error bars indicate the standard error of the mean. The black asterisk indicates that the treatment means are significantly different from the fungicide Nystatin at p≦0.05. The grey asterisk (M1, M2, M3, M4, M5, M6 and M7) indicates that the treatment means are significantly different from the fungicide Amphotericin at p≦0.05. (F) depicts quantification of the inhibitory effect of the endophyte strain M6 or fungicide control (amphotericin B or nystatin at concentrations of 5 and 10 μg/ml, respectively), on the growth of diverse fungal pathogens in vitro. For these experiments, n=3. The error bars indicate the standard error of the mean. The black asterisk (all M6 series except Penicillium sp.) indicates that the treatment means are significantly different from the fungicide Nystatin at p≦0.05. The grey asterisk indicates that the treatment means are significantly different from the fungicide Amphotericin at p≦0.05

FIG. 2 depicts in vitro interactions between the M6 endophyte and F. graminearum. (A) is a cartoon representation of the experimental methodology where F. graminearum (pink) and each endophytic extract (orange) or the buffer control were co-incubated for 24 hours on microscope slides coated with PDA; F. graminearum hyphae were then stained with the vitality stain, neutral red and Evans blue. Shown are representative pictures (n=3) of the interactions of F. graminearum. (B)-(D) show Hyphae of F. graminearum stained with neutral red while (E)-(F) show Hyphae of F. graminearum stained with Evans blue. (C), (D) and (F) show disintegrated hyphae of F. graminearum adjacent to M6, (B) and (E) show Hyphae of F. graminearum when grown away from M6. Shown are representative pictures (n=3).

FIG. 3 depicts the methodology for corn greenhouse trials. (A) shows corn seeds, (B) shows growing seeds on wetted paper towel, (C) shows the randomized block design, and (D) shows a picture of the greenhouse.

FIG. 4 depicts suppression of Giberella ear rot in corn by comparing to Fusarium challenged plants and proline, a commercial fungicide. (A)-(C) correspond to greenhouse trial 1. (A) shows representative pictures of corn ears treated with Fusarium, Proline and M6, (B) is a graphical representation of percent of infection, (C) is a graphical representation of average yield in gram per ear. (D)-(F) correspond to greenhouse trial 2. (D) shows representative pictures of corn ears treated with Fusarium, Proline and M6, (E) is a graphical representation of percent of infection, and (F) is a graphical representation of average yield in gram per ear. (G) shows quantification of the effect of seed coating versus foliar spray on GER suppression in two greenhouse trials. (H) Quantification of the effect of each treatment on average grain yield per plant in two greenhouse trials. For all measurements, n=20 per treatment (n=10 for both controls). The whiskers indicate the range of data points. The black asterisk indicates that the treatment means were significantly different from the Fusarium only treatment at p≦0.05. The grey asterisk (M6) indicates that the treatment means were significantly different from prothioconazole fungicide (Proline) treatment at p≦0.05.

FIG. 5 depicts suppression of Fusarium Head blight in Wheat by comparing to Fusarium challenged plants and proline, a commercial fungicide. (A) is a picture of a healthy wheat seed compared to (B), which is a diseased seed showing wrinkled surface, pale colour and growth of Fusarium hyphae at the tip of the seed. (C) is a picture of the greenhouse showing wheat plants. (D) is a graphical representation of percent of infection, (E) is a graphical representation of average yield in gram per plant for greenhouse trial 1, (F) is a graphical representation of percent of infection, and (G) is a graphical representation of average yield in gram per plant in greenhouse 2.

FIG. 6 depicts EZ::TN™ Transposon mutagenesis to discover M6 genes responsible for anti-Fungal Activity. (A) shows knockout mutants growing on Kanamycinmedia, (B) depicts agar diffusion anti-Fusarium screening showing some candidate knockout mutants that lost the anti-Fusarium activity and (C) shows E. coli transformed with the rescued candidate genes grown on Kanamycin. (D) depicts the quantification of the inhibitory effect of insertion mutants or wild type of strain M6 on the growth of F. graminearum in vitro. For these experiments, n=3. The error bars indicate the standard error of the mean. The black asterisk indicates that the treatment means are significantly different from the wild type at p≦0.05.

FIG. 7 depicts the validation of the candidate genes characterized in the in vitro random transposon mutagenesis in the greenhouse for suppression of Giberella ear rot in Corn comparing to the wild type M6 (Trial 1). Pictures from (A-G) show some representative ears from each treatment as indicated. (H) is a graphical representation of Giberella ear rot suppression by wild type and candidate knockout mutants.

FIG. 8 depicts the validation of the candidate genes characterized in the in vitro random transposon mutagenesis in the greenhouse for suppression of Giberella ear rot in Corn compared to the wild type M6 (Trial 2). Pictures from (A-G) show some representative ears from each treatment as indicated. (H) is a graphical representation of Giberella ear rot suppression by wild type and candidate knockout mutants.

FIG. 9 compares suppression of Giberella ear rot in corn by M6 to four potential candidate bacterial endophytes isolated from diverse corn genotypes. (A) and (B) correspond to greenhouse trial 1. (A) is a graphical representation of percent of infection and (B) is a graphical representation of average yield in gram per ear. (C) and (D) correspond to greenhouse trial 2. (C) is a graphical representation of percent of infection and (D) is a graphical representation of average yield in gram per ear.

FIG. 10 compares suppression of Fusarium Head Blight in Wheat by M6 to four potential candidate bacterial endophytes isolated from diverse corn genotypes. (A) and (B) correspond to greenhouse trial 1. (A) is a graphical representation of percent of infection and (B) is a graphical representation of average yield in gram per plant. (C) and (D) correspond to greenhouse trial 2. (C) is a graphical representation of percent of infection and (D) is a graphical representation of average yield in gram per plant.

FIG. 11 shows in planta colonization by GFP-M6. In planta colonization by GFP-tagged M6 was visualized using Leica confocal software. (A)-(D) depict GFP-M6 inside the shoot of one week old corn seedlings and (E)-(G) depict GFP-M6 inside the shoot of one week old finger millet seedlings.

FIG. 12 shows electron microscopy images of strain M6. (A) A picture of M6 taken by electron scanning microscopy. (B) A picture of M6 taken by electron transmission microscopy.

FIG. 13 shows testing for the ability of endophyte strain M6 to reduce DON mycotoxin accumulation in maize and wheat grains during storage. DON measurements after storage of grains from: (A) maize greenhouse trials (summer 2012, 2013), and (B) wheat greenhouse trials (summer 2013). For all trials, n=3 pools of seeds. The black asterisk indicates that the treatment means were significantly different from the Fusarium only treatment at p≦0.05. The grey asterisk (M6, Trial 1) indicates that the treatment means were significantly different from the prothioconazole fungicide (Proline) treatment at p≦0.05.

FIG. 14 shows gene expression analysis using real time-PCR. (A-F) Quantification of the ratio of gene expression in each mutant as indicated. For this experiment, results were pooled from three independent replicates, n=3 in each trial. White bars represents controls (blank and chitin treatment) while black bars represent induction pattern with the addition of Fusarium mycelium. The error bars indicate the standard error of the mean. The black asterisk indicates that the treatment means are significantly different from the blank at p≦0.05.

FIG. 15 shows biochemical detection of the candidate anti-fungal compound, phenazines, in strain M6. (A-D) Combined ion chromatogram/mass spectrum for candidate phenazine derivatives detected in the active anti-Fusarium broth of strain M6 as indicated.

FIG. 16 shows real time-PCR analysis for genes that showed minimal induction by Fusarium mycelium. (A-G) Quantification of the ratio of gene expression in each mutant as indicated. For this experiment, results were pooled from three independent replicates, n=3 in each trial. White bars represents controls (blank and chitin treatment) while black bars represent induction pattern with the addition of Fusarium mycelium. The error bars indicate the standard error of the mean. The black asterisk indicates that the treatment means are significantly different from the blank at p≦0.05.

### DETAILED DESCRIPTION

The present disclosure relates to a previously unidentified bacterial endophyte that can be isolated from finger millet and which is capable of inhibiting growth of fungal pathogens including Fusarium graminearum. The isolated bacterial endophyte is a novel Enterobacter species.

The present disclosure also relates to novel genes identified in the bacterial endophyte that are required for its antifungal activity.

### I. Definitions

The term “endophyte” as used herein refers to a class of microbial symbionts that reside within host plant roots, stems and/or leaves.

The term “inoculating a plant” with an endophyte, for example, as used herein refers to applying, contacting or infecting a plant (including its roots, stem, leaves or seeds) with an endophyte or a composition comprising an endophyte. The term “inoculated plant” refers to a plant to which an endophyte or a composition comprising an endophyte has been applied or contacted.

The present invention contemplates the use of “isolated” endophyte. As used herein, an isolated endophyte is an endophyte that is isolated from its native environment, and carries with it an inference that the isolation was carried out by the hand of man. An isolated endophyte is one that has been separated from at least some of the components with which it was previously associated (whether in nature or in an experimental setting). The term “isolated Enterobacter species” as used herein refers to a bacterial endophyte isolated from finger millet and having anti-Fusarium activity.

The term “mutant of the isolated Enterobacter species” as used herein refers to a bacterial strain that has undergone a mutation in its genetic code as compared to the isolated Enterobacter species, such as might be artificially created to enhance plant growth-related capabilities, to track the strain in the plant, or to track the strain in the environment to ensure consistency and provenance.

In some embodiments, the invention uses endophytes that are heterologous to a seed or plant in making synthetic combinations or agricultural formulations. An endophyte is considered heterologous to the seed or plant if the seed or seedling that is unmodified (e.g., a seed or seedling that is not treated with a bacterial endophyte population described herein) does not contain detectable levels of the endophyte. For example, the invention contemplates the synthetic combinations of plants, seeds or seedlings of agricultural plants (e.g., agricultural grass plants) and an endophyte population, in which the endophyte population is heterologously disposed on the exterior surface of or within a tissue of the agricultural plant, seed or seedling in an amount effective to colonize the plant. An endophyte is considered heterologously disposed on the surface or within a plant (or tissue) when the endophyte is applied or disposed on the plant in a number that is not found on that plant before application of the endophyte. For example, a bacterial endophytic population that is disposed on an exterior surface or within the seed can be an endophytic bacterium that may be associated with the mature plant, but is not found on the surface of or within the seed. As such, an endophyte is deemed heterologous or heterologously disposed when applied on the plant that either does not naturally have the endophyte on its surface or within the particular tissue to which the endophyte is disposed, or does not naturally have the endophyte on its surface or within the particular tissue in the number that is being applied.

The term “progeny of the isolated Enterobacter species” as used herein refers to all cells deriving from the isolated Enterobacter species.

The term “plant” as used herein includes any member of the plant kingdom that can be colonized by a bacterial endophyte. In one embodiment, the plant is an agricultural plant including, without limitation, finger millet, maize, wheat, sorghum and barley. As used herein, the term “plant” includes parts of a plant such as roots, stems, leaves and/or seeds that can be colonized by a bacterial endophyte.

The term “inhibiting fungal growth in a plant” as used herein means decreasing amount of fungal growth on a plant, decreasing the speed of fungal growth on a plant, decreasing the severity of a fungal infection in a plant, decreasing the amount of diseased area of a plant, decreasing the percentage of infected seeds, decreasing the percentage of apparent fungal infection of a plant and/or treating or preventing fungal growth in a plant.

The term “yield” refers to biomass or seed or fruit weight, seed size, seed number per plant, seed number per unit area, bushels per acre, tons per acre, kilo per hectare, and/or carbohydrate yield.

The term “promoting plant growth” as used herein means that the plant or parts thereof (such as seeds and roots) have increased in size or mass compared to a control plant, or parts thereof, that has not been inoculated with the endophyte or as compared to a predetermined standard.

The term “symbiosis” and/or “symbiotic relationship” as used herein refer to a mutually beneficial interaction between two organisms including the interaction plants can have with bacteria. Similarly, the term “symbiont” as used herein refers to an organism in a symbiotic interaction.

The term “sequence identity” as used herein refers to the percentage of sequence identity between two nucleic acid and/or polypeptide sequences. To determine the sequence identity of two nucleic acid sequences, the sequences are aligned for optimal comparison purposes (e.g., gaps can be introduced in the sequence of a first nucleic acid sequence for optimal alignment with a second nucleic acid sequence). The nucleic acid residues at corresponding nucleic acid positions are then compared. When a position in the first sequence is occupied by the same nucleotide as the corresponding position in the second sequence, then the molecules are identical at that position. The sequence identity between the two sequences is a function of the number of identical positions shared by the sequences (i.e., % identity=number of identical overlapping positions/total number of positions×100%). In one embodiment, the two sequences are the same length. The determination of sequence identity between two sequences can also be accomplished using a mathematical algorithm.

A “synthetic combination” includes a combination of a plant, such as an agricultural plant, and an endophyte. The combination may be achieved, for example, by coating the surface of the seed of a plant, such as an agricultural plant, or plant tissues with an endophyte.

As used herein, the terms “a” or “an” in relation to an object mean a representative example from a collection of that object.

### II. Isolated Enterobacter Strain M6

Endophytes, microbes that live inside a plant without causing disease, can confer beneficial traits to their host such as promoting health or protecting against specific host pathogens. Bacterial endophyte cultures were isolated from samples of finger millet plants to identify endophytes that could act as biocontrols for the fungus Fusarium. Once isolated from the plants, endophyte bacteria were identified using 16S rRNA sequencing.

The inventors isolated one species that they determined to be from the bacterium Enterobacter. The isolated species is also referred to herein as endophyte M6 or strain M6 and has a 16S rRNA gene sequence that comprises the nucleotide sequence set forth in SEQ ID NO: 1.

Accordingly, in a first aspect, the disclosure provides an isolated Enterobacter species, and its progeny thereof, or an isolated culture thereof, or a mutant thereof having the ability to inhibit fungal growth. In another aspect, the disclosure provides an isolated Enterobacter species, and its progeny thereof, or an isolated culture thereof, or a mutant thereof having the ability to inhibit Fusarium, optionally Fusarium graminearum growth.

The 16S rRNA gene is widely used for the classification and identification of microbes. It is well known in the art that bacteria of the same species need not share 100% sequence identity in the 16S rRNA sequences. Accordingly, in one aspect of the disclosure, the isolated Enterobacter species has a 16S rRNA gene comprising a nucleotide sequence that has more than 96% sequence identity to the sequence set forth in SEQ ID NO:1. In another aspect, the isolated Enterobacter species has a 16S rRNA gene comprising a nucleotide sequence has more than 97%, 97.5%, 98%, 98.5%, 99%, 99.5% or 99.9% sequence identity to the sequence set forth in SEQ ID NO:1.

In another aspect, the isolated Enterobacter species has a 16S rRNA gene comprising at least 100, 200, 300, 400, 450, 500 or 525 consecutive nucleotides of the sequence set forth in SEQ ID NO: 1.

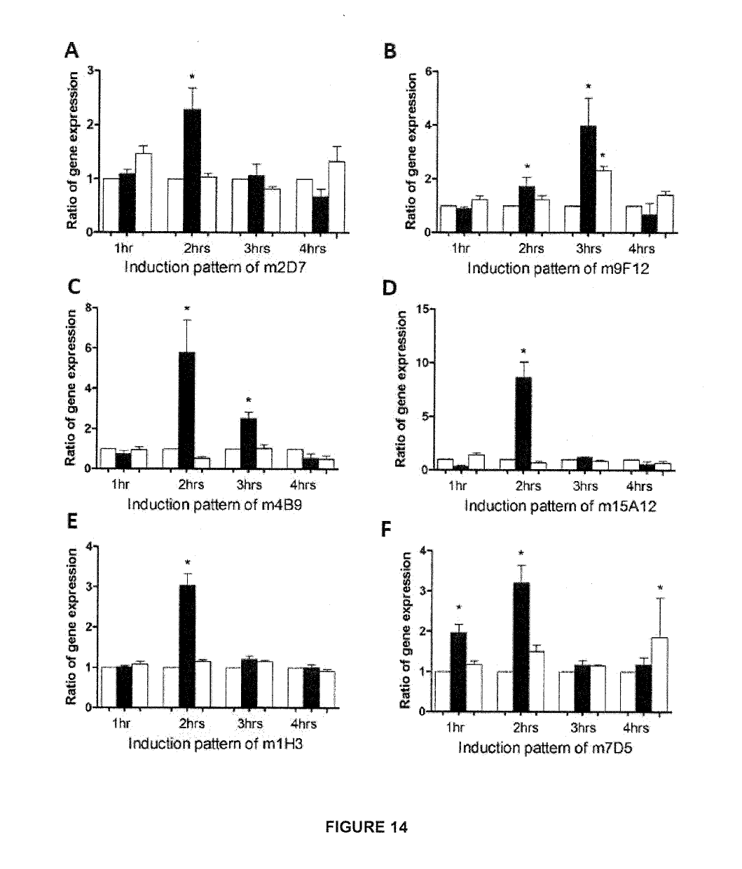

The endophyte M6 or strain M6 as described herein can readily be obtained from samples of finger millet plants such as by using the methods described in the Example 1. Confirmation of the identity of the bacterial endophyte can be performed by sequencing the 16S rRNA gene of the isolates and comparing the sequence to that of the sequence set forth in SEQ ID NO: 1. As shown in FIG. 12, the bacterial endophyte M6 described herein also exhibits a characteristic rod-like shape. In one embodiment, the bacterial endophyte M6 also exhibits a characteristic pattern of induction as a result of addition of Fusarium mycelium of one or more of genes m2D7, m9F12, m4B9, m115A12, m1H3 and m5D7 as shown in FIG. 14 and Example 3. In some embodiments, the induction is at least 1.5 fold. In some embodiments, the induction is at least 2 fold. In some embodiments, the induction is at least 2.5 fold.

### III. Compositions Comprising the Isolated Bacterial Endophyte

Compositions for inoculating the plants with the isolated bacterial endophyte described herein are also disclosed. In one aspect, the disclosure provides an inoculating composition, comprising an Enterobacter species having a 16S rRNA gene comprising a nucleotide sequence that has more than 96% sequence identity to the sequence set forth in SEQ ID NO:1 or its progeny, or mutants thereof, and optionally a carrier. The composition may be applied to any part of the plant including roots, leaves, stems or seeds.

As used herein, the term “carrier” refers to the means by which the bacterial endophyte is delivered to the target plant. Carriers that may be used in accordance with the present disclosure include oils, polymers, plastics, wood, gels, colloids, sprays, drenching means, emulsifiable concentrates and so forth. The selection of the carrier and the amount of carrier used in a composition or formulation may vary and depends on several factors including the specific use and the preferred mode of application.

The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in a composition of the invention. Water-in-oil emulsions can also be used to formulate a composition that includes the purified bacterial population (see, for example, U.S. Pat. No. 7,485,451, which is incorporated herein by reference in its entirety). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, plant parts, sugar cane bagasse, hulls or stalks from grain processing, ground plant material or wood from building site refuse, sawdust or small fibers from recycling of paper, fabric, or wood. Other suitable formulations will be known to those skilled in the art.

In one embodiment, the composition comprises a suspension of the isolated bacterial endophyte and a seed coating agent as carrier. Optionally, the seed coating agent is polyvinyl pyrrolidine (PVP). In one example, 500 μL of bacterial suspension, optionally 10 μl to 1 mL of bacterial suspension, is mixed with 10 ml of PVP, optionally 1 ml to 100 ml PVP.

In one embodiment, the composition includes at least one member selected from the group consisting of a tackifier, a microbial stabilizer, a fungicide, an antibacterial agent, an herbicide, a nematicide, an insecticide, a plant growth regulator, a rodenticide, a dessicant, and a nutrient.

In one embodiment, the formulation can include a tackifier or adherent. Such agents are useful for combining the bacterial population of the invention with carriers that can contain other compounds (e.g., control agents that are not biologic), to yield a coating composition. Such compositions help create coatings around the plant or seed to maintain contact between the microbe and other agents with the plant or plant part. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. Other examples of adherent compositions that can be used in the synthetic preparation include those described in EP 0818135, CA 1229497, WO 2013090628, EP 0192342, WO 2008103422 and CA 1041788, each of which is incorporated herein by reference in its entirety.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some cases, it is advantageous for the formulation to contain agents such as a fungicide, an antibacterial agent, an herbicide, a nematicide, an insecticide, a plant growth regulator, a rodenticide, or a nutrient. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

In one embodiment of the disclosure, the composition is in a fluid form suitable for spray application or seed coating. In the liquid form, for example, solutions or suspensions, the bacterial endophytic populations of the present invention can be mixed or suspended in water or in aqueous solutions. Suitable liquid diluents or carriers include water, aqueous solutions, petroleum distillates, or other liquid carriers.

In another embodiment, said composition is in a paste-like form. In still another embodiment, the composition is in a substantially dry and powdered form for dusting. Solid compositions can be prepared by dispersing the bacterial endophytic populations of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as nonionic, anionic, amphoteric, or cationic dispersing and emulsifying agents can be used.

The composition is optionally applied as a foliar spray. In another embodiment, the composition is applied to seeds, as a seed coating. In yet another embodiment, the composition is applied both as a foliar spray and a seed coating.

In another embodiment, the composition comprises a suspension of the bacterial endophyte in liquid broth media (for example, bacto-yeast, tryptone and sodium chloride) for spray application. The bacterial density of the spray is optionally OD600=0.5 or OD600=0.1 to 0.8.

The formulations comprising the bacterial endophytic population of the present invention typically contains between about 0.1 to 95% by weight, for example, between about 1% and 90%, between about 3% and 75%, between about 5% and 60%, between about 10% and 50% in wet weight of the bacterial population of the present invention. It is preferred that the formulation contains at least about 10³ CFU per ml of formulation, for example, at least about 10⁴, at least about 10⁵, at least about 10⁶, at least 10⁷ CFU, at least 10⁸ CFU per ml of formulation.

In one embodiment, the composition can inhibit fungal growth. One of skill in the art can readily determine the amount or concentration of the composition that can be applied to the plant or plant seed to inhibit fungal growth. For example, the bacterial density of the inoculate can range from an OD600 of 0.1 to 0.8. In one embodiment, the bacterial density of the inoculate at OD600 is 0.5, optionally approximately 0.5.

Also provided are synthetic combinations of the isolated bacterial endophyte described herein in association with a plant. In one embodiment, the bacterial endophyte reside within the seeds, roots, stems and/or leaves of the plant as an endophyte. Optionally, the plant may be a plant seed or seedling. In one embodiment, the bacterial endophyte is heterologous to the microbial population of the plant. For example, synthetic combination may be a bacterial strain having antifungal properties as described herein which has been artificially inoculated on a plant that does not naturally harbor or contain the bacterial endophyte. In one embodiment, the plant is an agricultural plant other than millet. In one embodiment, the agricultural plant is a cereal. In one embodiment, the cereal is maize, wheat, sorghum or barley.

### IV. Methods and Uses of Endophyte M6

### 1. Inhibiting Fungal Growth

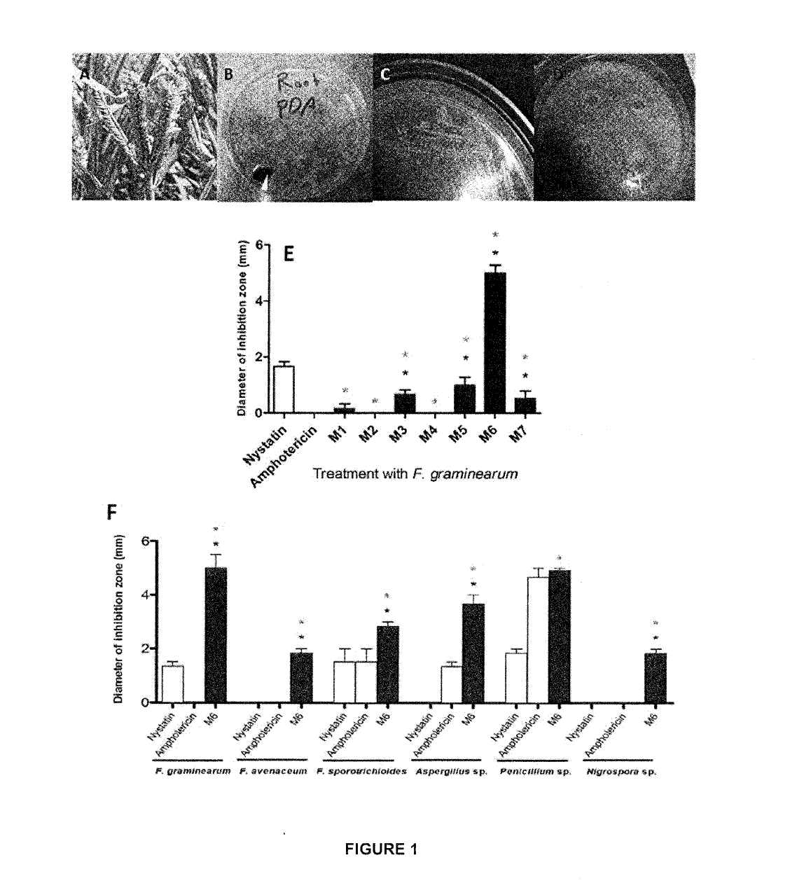

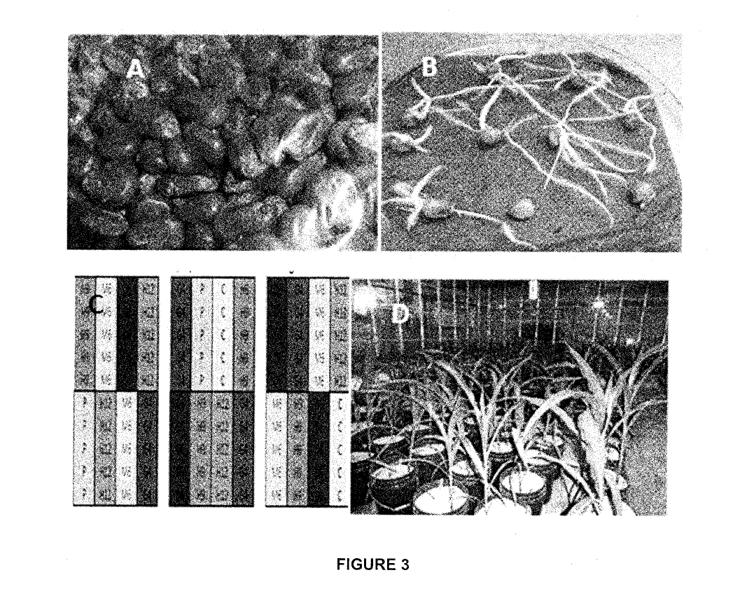

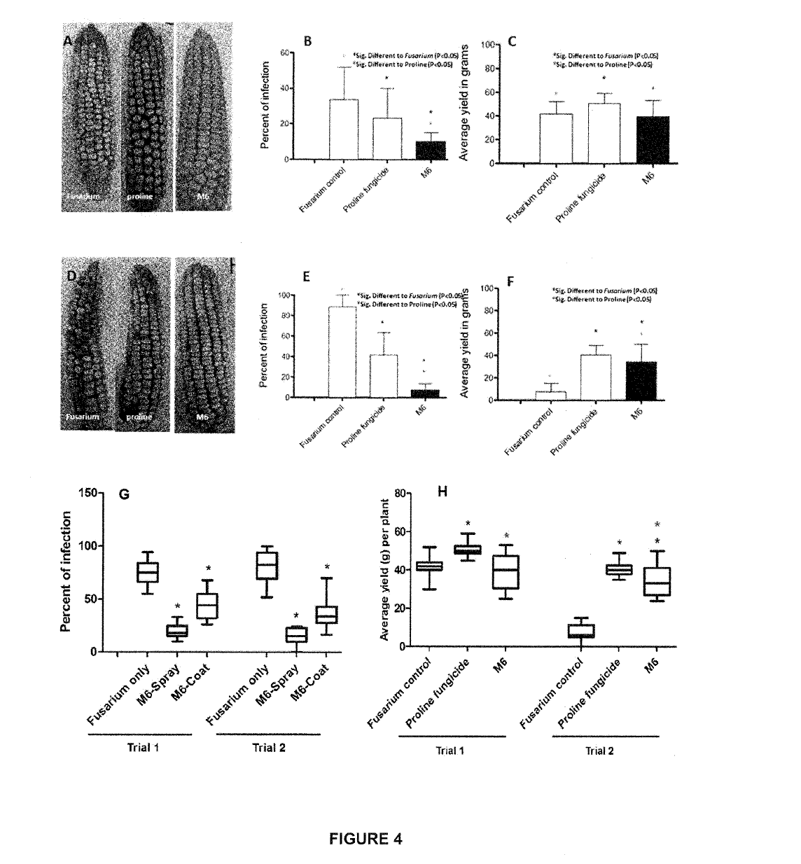

It is shown herein that bacterial endophyte M6 has antifungal activity. For example, bacterial endophyte M6 is able to suppress the growth of F. graminearum as shown in FIG. 1(D). M6 has also been shown to inhibit the growth of the following crop fungi: Aspergillus flavus, Aspergillus niger, Fusarium lateritium, Fusarium avenaceum, Fusarium sporotrichoides, Fusarium graminearum, Paraconiothyrium brasiliense, Penicillium expansum, Nigrospora oryzae and Trichoderma hamatum. Maize plants treated with M6 also showed a reduction in the severity of Gibberalla ear rot caused by F. graminearum (see for example FIGS. 3 and 4).

Therefore, in an aspect, the disclosure provides a method of preventing or inhibiting fungal growth on a plant, comprising inoculating a plant with the isolated bacterial endophyte described herein.

In another embodiment, the disclosure provides a use of the isolated bacterial endophyte to prevent or inhibit fungal growth on a plant.

Various fungi can be inhibited by the bacterial endophyte described herein. In one aspect of the disclosure, the fungus belongs to class Eurotiomycetes or to class Sordariomycetes. In another aspect of the disclosure, the fungus belongs to order Eurotiales, Hypocreales, or Trichosphaeriales. In yet another aspect of the disclosure, the fungus belongs to family Nectriaceae, Trichocomaceae or Hypocreaceae. In another aspect of the disclosure, the fungus belongs to one of the following genera: Fusarium and Aspergillus. In another aspect of the disclosure, the fungus is Aspergillus flavus, Aspergillus niger, Fusarium lateritium, Fusarium avenaceum, Fusarium sporotrichoides, Fusarium graminearum, Paraconiothyrium brasiliense, Penicillium expansum, Nigrospora oryzae and/or Trichoderma hamatum.

“Inhibiting fungal growth” includes, but is not limited to, decreasing the amount of fungal growth on a plant, decreasing the speed of fungal growth on a plant, decreasing the severity of a fungal infection on a plant, decreasing the amount of diseased area of a plant, decreasing the percentage of infected seeds and/or decreasing the percentage of apparent fungal infection of a plant. Any of the above criteria can be decreased by at least 5%, 7.5%, 10%, 15%, 20%, 25%, 30%, 40%, 50%, 75%, 100%, 150% or 200% in an inoculated plant compared to a non-inoculated plant.

“Inhibiting fungal growth” also includes preventing fungal growth.

“Inhibiting fungal growth on a plant” can result in improved growth of the inoculated plant.

Determining an improvement in plant growth using the bacterial endophyte described herein can be assessed in a number of ways. For example, the size or weight of the entire plant or a part thereof (such as seeds or roots) can be measured. In an embodiment, the average mass of an inoculated plant is increased at least 5%, 7.5%, 10%, 15%, 20%, 25%, 30%, 40%, 50%, 75%, 100%, 150% or 200% fresh weight or dry weight. In another embodiment, the average mass of the seeds of an inoculated plant is increased at least 5%, 7.5%, 10%, 15%, 20%, 25%, 30%, 40%, 50%, 75%, 100%, 150% or 200% fresh weight or dry weight. In still another embodiment, the endophyte-associated plant exhibits at least 4%, 5%, 6%, 7%, 8%, 9%, 10%, 15%, 20%, 25%, 30%, 40%, 50%, 75%, 100%, 150% or 200% or more fruit or grain yield, than the reference agricultural plant grown under the same conditions.

The bacterial endophyte described herein may also be used as prophylactic agent to decrease the chance of a fungal infection from occurring in a plant.

The bacterial endophyte described herein may also be used for treating and/or preventing ear and/or kernel rot diseases in corn. In one aspect of the disclosure, the bacterial endophyte described herein is used to treat Gibberalla ear rot caused by F. graminearum in corn. Treating and/or preventing ear and/or kernel rot diseases in corn includes, but is not limited to, decreasing the amount of ear and/or kernel rot, decreasing the severity of ear and/or kernel rot, decreasing the amount of diseased area of a plant, decreasing the percentage of infected seeds and/or decreasing the percentage of apparent ear and/or kernel rot. Any of the above criteria can be decreased by at least 5%, 7.5%, 10%, 15%, 20%, 25%, 30%, 40%, 50%, 75%, 100%, 150% or 200% in an inoculated plant compared to a non-inoculated plant.

Treating and/or preventing ear and/or kernel rot diseases in corn can also result in improved growth of the inoculated corn plant. Improved growth of the corn plant can be assessed in a number of ways. For example, the size or weight of the entire plant or a part thereof (such as kernels) can be measured. Improved growth can also be measured by an increase in the speed of growth of the plant.

In an embodiment, the average mass of an inoculated plant is increased at least 5%, 7.5%, 10%, 15%, 20%, 25%, 30%, 40%, 50%, 75%, 100%, 150% or 200% fresh weight or dry weight. In another embodiment, the average mass of the kernels of an inoculated corn plant is increased at least 5%, 7.5%, 10%, 15%, 20%, 25%, 30%, 40%, 50%, 75%, 100%, 150% or 200% fresh weight or dry weight. In still another embodiment, the endophyte-associated plant exhibits at least 4%, 5%, 6%, 7%, 8%, 9%, 10%, 15%, 20%, 25%, 30%, 40%, 50%, 75%, 100%, 150% or 200% or more grain yield, than the reference agricultural plant grown under the same conditions.

The bacterial endophyte described herein may also be used for treating and/or preventing head blight diseases in plants such as wheat or barley. In one aspect of the disclosure, the bacterial endophyte described herein is used to treat head blight in plants caused by F. graminearum. Treating and/or preventing head blight in a plant includes, but is not limited to, decreasing the amount of head blight, decreasing the severity of head blight, decreasing the amount of diseased area of a plant, decreasing the percentage of infected seeds and/or decreasing the percentage of apparent head blight. Any of the above criteria can be decreased by at least 5%, 10%, 25%, 50%, 75%, 100%, 150% and 200% in an inoculated plant compared to a non-inoculated plant.

Treating and/or preventing head blight diseases in plants such as wheat or barley can also result in improved growth of the inoculated plant. Improved growth of the wheat or barley plant can be assessed in a number of ways. For example, the size or weight of the entire plant or a part thereof can be measured. Improved growth can also be measured by an increase in the speed of growth of the plant.

In an embodiment, the average mass of an inoculated wheat or barley plant is increased at least 5%, 7.5%, 10%, 15%, 20%, 25%, 30%, 40%, 50%, 75%, 100%, 150% or 200% fresh weight or dry weight. In another embodiment, the percentage of infected seeds in an inoculated wheat or barley plant is increased at least 5%, 7.5%, 10%, 15%, 20%, 25%, 30%, 40%, 50%, 75%, 100%, 150% or 200%. In still another embodiment, the endophyte-associated plant exhibits at least 4%, 5%, 6%, 7%, 8%, 9%, 10%, 15%, 20%, 25%, 30%, 40%, 50%, 75%, 100%, 150% or 200% or more grain yield, than the reference agricultural plant grown under the same conditions.

In one embodiment, the disclosure also provides a method for reducing the levels of deoxynivalenol (DON) in a plant during storage, comprising inoculating the plant with the bacterial endophyte or composition described herein. In one embodiment, the levels of DON in the plant are reduced relative to a plant that has not been inoculated with the bacterial endophyte or composition as described herein. In one embodiment, the level of DON in an inoculated plant can be decreased by at least 5%, 7.5%, 10%, 15%, 20%, 25%, 30%, 40%, 50%, 75%, 100%, 150% or 200% compared to a non-inoculated plant. For example, in one embodiment levels of DON in corn or wheat seeds are reduced by at least 5%, 7.5%, 10%, 15%, 20%, 25%, 30%, 40%, 50%, 75%, 100%, 150% or 200% compared to a non-inoculated seeds stored for at least 1 month, 2 months, 3 months or 6 months. Also provided is the use of a bacterial strain or composition as described herein for reducing the levels of DON in a plant during storage.

### 2. Inoculation Methods for Plants

It should be understood that the methods and uses described herein for plant inoculation apply to all methods and uses of the disclosure described, for example, for preventing or inhibiting fungal growth on a plant.

The plant can be inoculated with the bacterial endophytes described herein or a composition comprising the bacterial endophytes described herein, using techniques known in the art. For example, the bacterial endophytes may be applied to the roots of the plant, or to young germinated seedlings, or to ungerminated or germinated seeds.

The methods described herein can be applied to any plant in need thereof. It is known that bacterial endophytes readily colonize a wide diversity of plant species and thus inoculation with strains described herein will colonize a variety of plant species. In one embodiment, the plant is an agricultural plant or crop.

It is shown herein that M6 endophyte colonization occurs in finger millet, as in Example 1, as well as corn/maize, and wheat (see FIGS. 3-5). Thus, in one embodiment, the plant is a monocotyledonous plant. In another embodiment, the plant belongs to the Poacoea family. In another embodiments, the plant is a domesticated Poacoea, for example a cereal plant. In another embodiment, the agricultural plant is a finger millet plant. In another embodiment, the agricultural plant is a corn plant, a wheat plant, sorghum or a barley plant.

In yet other embodiments, the plant is a cereal such as rice, sugarcane, oats, pearl millet, rye, triticale or tef or a grass such as creeping bentgrass, Kentucky bluegrass, tall fescue, Bermudagrass or ryegrass.

### V. Novel Genes and Uses Thereof

Ten novel genes are shown herein to be required for the antifungal activity of endophyte M6.

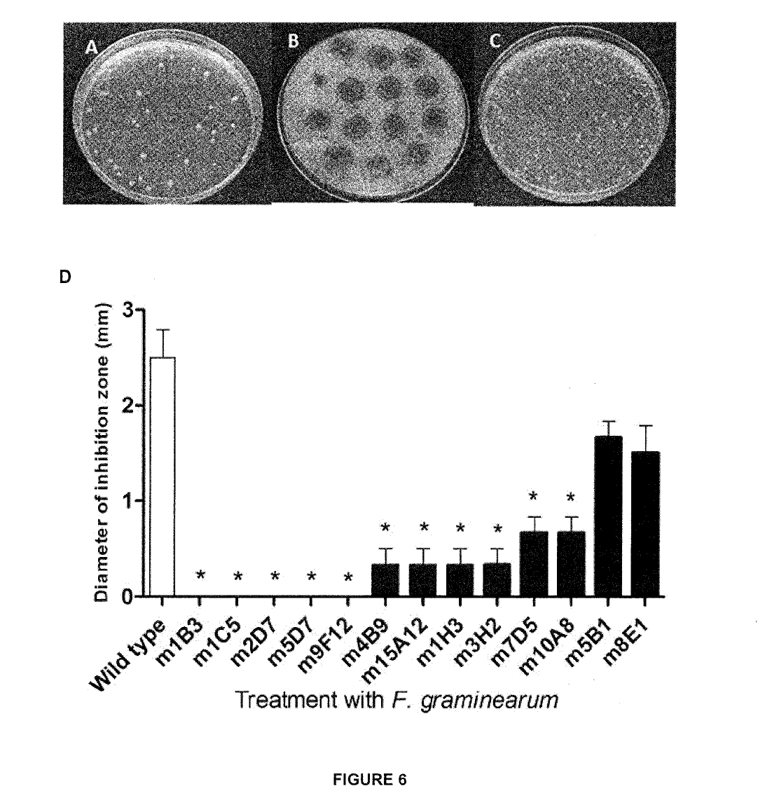

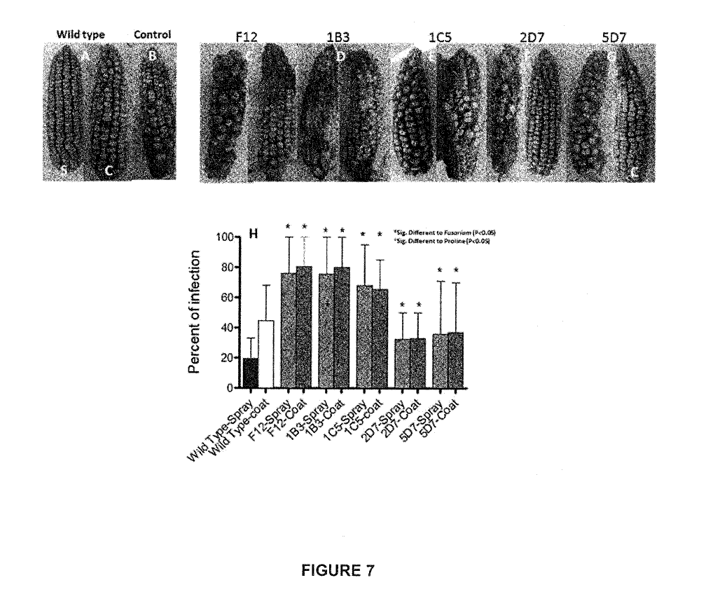

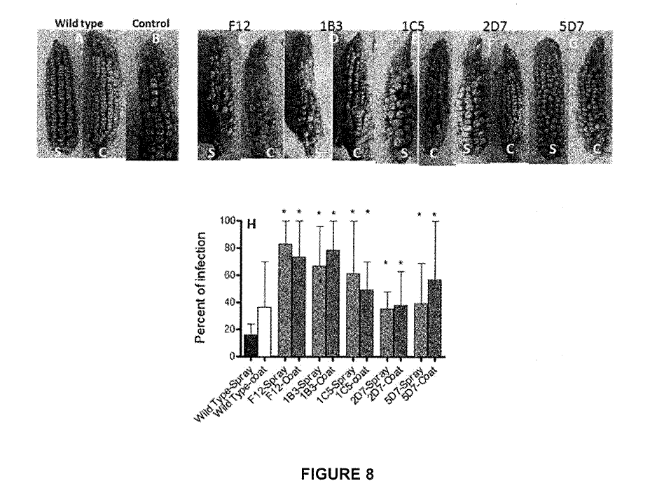

Using Tn5 transposon mutagenesis, ten mutants of endophyte M6 were identified that caused a loss of anti F. graminearum activity (see Table 1 and FIG. 6). Five of the mutants were tested in the greenhouse and were also shown to be necessary for suppression of Giberella ear rot in corn (see FIGS. 7 and 8).

TABLE 1
Genes involved in the antifungal activity of endophyte M6. Gene
products are predicted based on the best BLAST match against the M6
genome.

| | | Nucleotide | Amino Acid |
|---|---|---|---|
| Gene ID | Gene product | Sequence | Sequence |
| m1B3 | AraC transcriptional factor | SEQ ID NO: 2 | SEQ ID NO: 12 |
| m9F12 | long chain fatty acid ligase | SEQ ID NO: 3 | SEQ ID NO: 13 |
| m15A12 | Permease | SEQ ID NO: 4 | SEQ ID NO: 14 |
| m1C5 | outer membrane lipoprotein | SEQ ID NO: 5 | SEQ ID NO: 15 |
| m3H2 | Chitinase | SEQ ID NO: 6 | SEQ ID NO: 16 |
| m5D7 | LysR family transcriptional regulator YneJ | SEQ ID NO: 7 | SEQ ID NO: 17 |
| m4B9 | Colicin V production protein | SEQ ID NO: 8 | SEQ ID NO: 18 |
| m1H3 | Putative sensory histidine kinase YfhA | SEQ ID NO: 9 | SEQ ID NO: 19 |
| m10A8 | diguanylate cyclase/phosphodiesterase | SEQ ID NO: 10 | SEQ ID NO: 20 |
| | (GGDEF & EAL domains) with PAS/PAC | | |
| | sensor(s) | | |
| m22D7 | 4-hydroxyphenylacetate 3-monooxygenase | SEQ ID NO: 11 | SEQ ID NO: 21 |
| | (EC 1.14.13.3) | | |

Accordingly, in one aspect of the disclosure, an isolated gene comprising a nucleotide sequence that has at least 70%, 80%, 85%, 90%, 95%, 96%, 97%, 98%, or 99% sequence identity to any one of SEQ ID NOs:2-11, or the complement thereof, is provided. In another aspect of the disclosure, an isolated gene comprising or consisting of the nucleotide sequence set forth in any one of SEQ ID NOs:2-11, or the complement thereof, is provided. In one embodiment, the isolated gene encodes for a protein that is capable of conferring antifungal activity, optionally anti-Fusarium activity, to a bacteria and/or plant.

In another aspect of the disclosure, an isolated protein comprising an amino acid sequence that has at least 70%, 80%, 85%, 90%, 95%, 96%, 97%, 98%, or 99% sequence identity to any one of SEQ ID NOs:12-21 is provided. In another aspect of the disclosure, an isolated protein comprising or consisting of the amino acid sequence set forth in any one of SEQ ID NOs:12-21 is provided. In one embodiment, the protein that is capable of conferring antifungal activity, optionally anti-Fusarium activity, to a bacteria and/or plant.

In another aspect of the disclosure, a recombinant DNA construct comprising an isolated gene comprising a nucleotide sequence that has at least 70%, 80%, 85%, 90%, 95%, 96%, 97%, 98%, or 99% sequence identity to any one of SEQ ID NOs:2-11, or the complement thereof, is provided. In another aspect of the disclosure, a recombinant DNA construct comprising an isolated gene comprising or consisting of the nucleotide sequence set forth in any one of SEQ ID NOs:2-11, or the complement thereof, is provided.

In another aspect of the disclosure, a recombinant DNA construct comprising a nucleotide sequence encoding a protein that has at least 70%, 80%, 85%, 90%, 95%, 96%, 97%, 98%, or 99% sequence identity to any one of SEQ ID NOs:12-21 is provided. In another aspect of the disclosure, a recombinant DNA construct comprising a nucleotide sequence encoding a protein set forth in any one of SEQ ID NOs:12-21 is provided.

The novel genes described herein are useful for conferring antifungal activity to a bacteria and/or plant. In one embodiment, expression of at least one of the novel genes in a bacteria and/or plant cell results in increased antifungal activity of the bacteria and/or plant cell. In another embodiment, the increased antifungal activity is increased anti-Fusarium activity.

Accordingly, another aspect of the disclosure provides transformed plant cells, plants, and plant parts, comprising the nucleic acid molecules of the disclosure and methods of generating the transformed plant cells, plants, and plant parts. As used herein, the term “plant parts” includes any part of the plant including the seeds.

Another aspect of the disclosure provides transformed bacterial cells, comprising the nucleic acid molecules of the disclosure and methods of generating the bacterial cells.

Transformation is a process for introducing heterologous DNA into a bacterial cell, plant cell, plant or plant part. Transformed bacterial cells, plant cells, plants, and plant parts are understood to encompass not only the end product of a transformation process, but also transgenic progeny thereof. “Transformed”, “transgenic” and “recombinant” refer to a host organism such as a bacterial strain or a plant into which a heterologous nucleic acid molecule has been introduced. The nucleic acid molecule can be stably integrated into the genome of the host or the nucleic acid molecule can also be present as an extrachromosomal molecule.

Methods of transformation are well known in the art. In one aspect of the present disclosure, transformation comprises introducing into a bacterial cell, plant cell, plant, or plant part an expression construct comprising a nucleic acid molecule of the present disclosure to obtain a transformed bacterial cell, plant cell, plant, or plant part, and then culturing the transformed bacterial cell, plant cell, plant, or plant part. The nucleic acid molecule can be under the regulation of a constitutive or inducible promoter. The method can further comprise inducing or repressing expression of a nucleic acid molecule of a sequence in the plant for a time sufficient to modify the concentration and/or composition in the bacterial cell, plant cell, plant, or plant part.

In one aspect of the disclosure, the transformed bacterial cell, plant cell, plant, or plant part is resistant to infection by pathogenic fungi. In one embodiment, the pathogenic fungi is Fusarium, optionally F. graminearum. In another embodiment, the pathogenic fungi is selected from the group consisting of Aspergillus flavus, Aspergillus niger, Fusarium lateritium, Fusarium avenaceum, Fusarium sporotrichoides, Fusarium graminearum, Paraconiothyrium brasiliense, Penicillium expansum, Nigrospora oryzae and Trichoderma hamatum.

Various plants can be transformed with the genes described herein. In one embodiment, the plant is maize, wheat, sorghum or barley.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present application:

### EXAMPLES

### Example 1

A. Isolation of Bacterial Endophytes from Finger Millet and Antifungal Screening

A total of eight bacterial endophytes were isolated from different tissues of finger millet as described in FIG. 1 (A-C). The overnight culture of bacterial endophytes were used to screen for inhibition of growth of F. graminearum in vitro using the agar diffusion method. The endophytes were screened in three independent replicates. One candidate bacterial endophyte named M6 isolated from millet root was identified to suppress the growth of F. graminearum as illustrated in FIG. 1 (D).

### B. Anti-Fungal Target Spectrum of the Candidate Endophytes

The M6 endophyte that tested positive against F. graminearum was re-screened for activity against a diversity of other crop fungi using the agar diffusion. M6 inhibited the growth of six pathogens. The pathogens inhibited by M6 are Aspergillus flavus, Aspergillus niger, Fusarium lateritium, Fusarium avenaceum, Fusarium sporotrichoides, Fusarium graminearum, Paraconiothyrium brasiliense, Penicillium expansum, Nigrospora oryzae and Trichoderma hamatum.

C. Molecular Identification of Candidate Endophytic Bacteria Using 16S rDNA

Based on 16S rDNA sequence comparisons using BLAST searches to Genbank, the Candidate endophyte was identified as an Enterobacter species. Complete genome sequencing is currently under way.

### D. Microscopic Mechanisms of Action

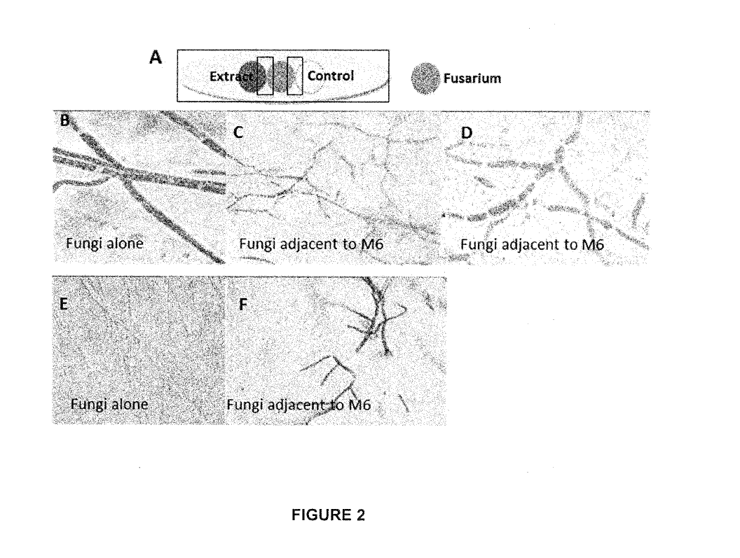

Microscope slides were used to view the in vitro interactions between pathogen and M6 endophyte using neutral red and Evans blue as vitality stains. Pictures were taken using a light microscope. There were 3-4 replicates for each slide. M6 caused disintegration of F. graminearum hyphae as shown in FIG. 2.

### E. Testing in Planta Activity of M6 to Suppress Gibberella Ear Rot in Corn (2 Replicates)

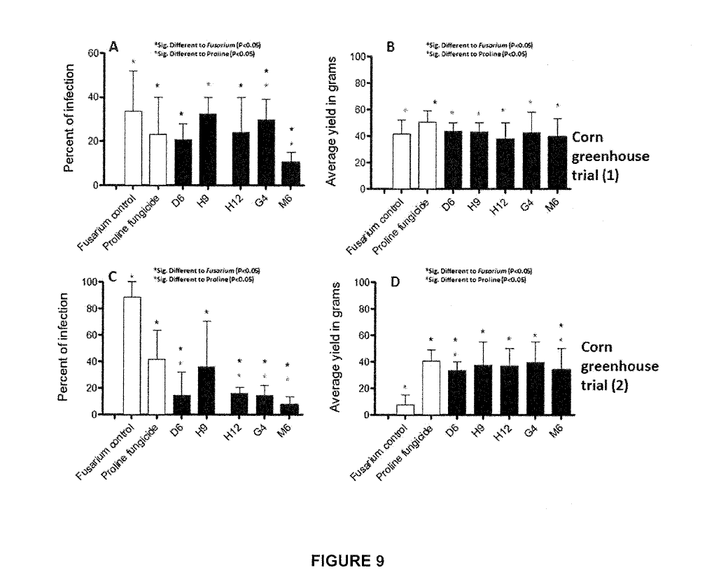

The candidate endophyte M6, was used to test for suppression of Gibberella ear rot disease caused by F. graminearum in maize plants in a greenhouse in two independent replicates (Summer, 2012 and 2013) using randomized block design (FIG. 3). Results were analyzed and compared by Mann-Whitney t-test (P<0.05). M6 treated plants showed remarkable reduction in disease severity compared to Fusarium challenged plants in both the two greenhouse replicates (FIG. 4). The ability of M6 to suppress Gibberella ear rot was compared to that caused by another four potential candidates endophytes (D6, H9, H12 and G4) isolated from diverse corn genotypes. M6 showed to have superior degree of disease suppression when compared to other Candidate endophytes (FIG. 9).

### F. Testing in Planta Activity of M6 to Suppress Fusarium Head Blight in Wheat (2 Replicates)

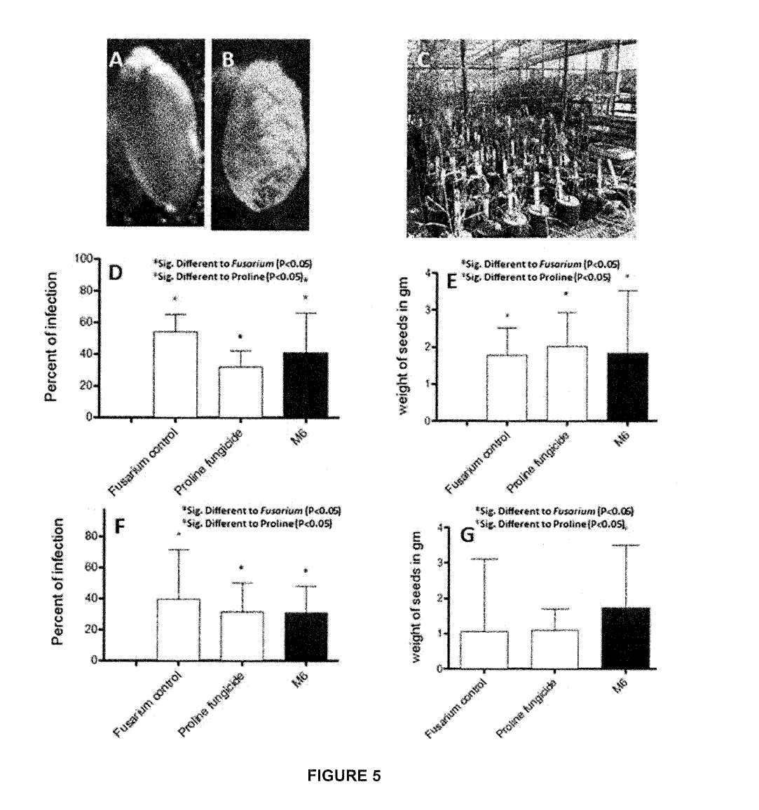

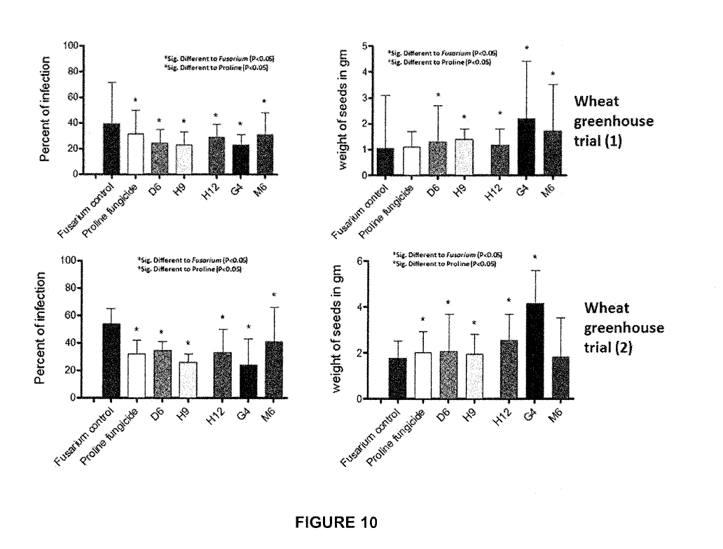

The candidate endophyte M6 was tested for suppression of Fusarium head blight disease caused by F. graminearum in Wheat plants in a greenhouse in a two independent replicates (Summer, 2013). Results were analyzed and compared by Mann-Whitney t-test (P<0.05). M6 treated plants showed reduction in disease severity compared to Fusarium challenged plants as shown in FIGS. 5 and 6. The ability of M6 to suppress Fusarium head blight was compared to that caused by another four potential candidates endophytes (D6, H9, H12 and G4) isolated from diverse corn genotypes (FIG. 10).

### Materials and Methods

A. Isolation of Bacterial Endophytes from Finger Millet and Antifungal Screening

Finger millet seeds were of commercial food grade, originating from Northern India. Plants were grown under semi-hydroponic conditions (on clay Turface MVP, Profile Products, Buffalo Grove, Ill., USA) in 22.5 L pales placed in the field (Arkell Field Station, Arkell, ON, Canada, GPS: 43° 39′ N, 80° 25′ W, and 375 m above sea level) during the summer of 2012 and irrigated with the following nutrient solution: urea (46% N content), superphosphate (16% P205), muriate of potash (60% K20), magnesium Epsom salt (16% MgO, 13% S), and Plant-Prod Chelated Micronutrient Mix (3 g/L, Plant Products, Catalog #10047, Brampton, Canada) consisting of Fe (2.1 ppm), Mn (0.6 ppm), Zn (0.12 ppm), Cu (0.03 ppm), B (0.39 ppm) and Mo (0.018 ppm). Six tissue pool sets (3 sets of: 5 seeds, 5 shoots and 5 root systems from pre-flowering plants) were surface sterilized as follows: samples were washed in 0.1% Triton X-100 detergent for 10 min with shaking; the detergent was decanted, 3% sodium hypochlorite was added (10 min twice for seeds; 20 min twice for roots), followed by rinsing with autoclaved, distilled water, washing with 95% ethanol for 10 min; and finally the samples were washed 5-6 times with autoclaved, distilled water. Effective surface sterilization was ensured by inoculating the last wash on PDA agar plates at 25° C.; all washes showed no growth. Tissues were ground directly in LB liquid media in a sterilized mortar and pestle, then 50 μl suspensions were plated onto 3 types of agar plates [LB, Potato Dextrose Agar (PDA) and Biolog Universal Growth [19] media (Catalog #70101, Biolog, Inc, Hayward, Calif., USA)]. Plates were incubated at 25° C. or 30° C. for 2-7 days. A total of 8 bacterial colonies were selected and purified by repeated culturing on fresh media.

The overnight culture of bacterial endophytes were used to screen for inhibition of growth of Fusarium graminearum in vitro (obtained from Agriculture and Agrifood Canada, Guelph, ON) using the agar diffusion method.

Briefly, the bacterial endophytes were grown in liquid broth (LB) overnight then centrifuged for 5 min, re-suspended in PBS buffer to an OD600=0.4-0.6 (Genesys 20, Thermoscientific). F. graminearum was grown for 24-48 h (25° C., 100 rpm) in liquid PDA media, then mycelia was added to melted, cooled PDA media (1 ml of fungus into 100 ml of media), mixed and poured into Petri dishes (100 mm×15 mm), then allowed to re-solidify. Wells (11 mm diameter) were created in this pathogen-embedded agar by puncturing with sterile glass tubes, into which the endophyte cultures were applied (200 μl per well). The agar plates were incubated at 30° C. for 48 h in darkness. The radius of each zone of inhibition was measured. The fungicides Amphotericin B (Catalog #A2942, Sigma Aldrich, USA) and Nystatin (Catalog #N6261, Sigma Aldrich, USA) were used as positive controls, and LB was used as a negative control. The endophytes were screened in three independent replicates.

### B. Anti-Fungal Target Spectrum of the Candidate Endophytes

The candidate endophyte that tested positive against F. graminearum was re-screened for activity against a diversity of other crop fungi using the agar diffusion method (described above) to characterize the activity spectrum of this endophyte. The crop pathogens tested included: Alternaria alternate, Alternaria arborescens, Aspergillus flavus, Aspergillus niger, Bionectria ochroleuca, Davidiella (Cladosporium) tassiana, Diplodia pinea, Diplodia seriata, Epicoccum nigrum, Fusarium lateritium, Fusarium sporotrichioides, Fusarium avenaceum (Gibberella avenacea, two isolates), Nigrospora oryzae, Nigrospora sphaerica, Paraconiothyrium brasiliense, Penicillium expansum, Penicillium afellutanum, Penicillium olsonii, Rosellinia corticium, Torrubiella confragosa, Trichoderma hamatum and Trichoderma longibrachiatum.

C. Molecular Identification of Candidate Endophytic Bacteria Using 16S rDNA

For molecular taxonomic identification of endophytic bacteria, a standard protocol was used (Johnston-Monje & Raizada, 2011). Bacterial genomic DNA was extracted using genomic DNA extaction kit (GenElute Bacterial Genomic DNA kit, NA2110-1KT, Sigma) and quantified using a Nanodrop machine (Thermo Scientific, USA). The extracted DNA was used to amplify 16S rDNA using PCR. A PCR master mix (20 μl) was made as follows: 50 ng/μl DNA, 2.5 μl Standard Taq Buffer (10×) (New England Biolabs), 0.5 μl of 25 mM dNTP mix, 1 μl of 10 mM 1492r primer with sequence GGTTACCTTGTTACGACTT (SEQ ID NO: 22), 1 μl of 10 mM 799f primer with sequence AACMGGATTAGATACCCKG (SEQ ID NO: 23, 0.25 μl of 50 mM MgCl2, 0.25 μl of Standard Taq (10 U/μl, New England Biolabs), and double distilled water up to 20 μl total. PCR amplification conditions was: 96° C. for 3 min, followed by 35 amplification cycles (94° C. for 30 sec, 48° C. for 30 sec, 72° C. for 1:30 min), and a final extension at 72° C. for 7 min, using a PTC200 DNA Thermal Cycler (MJ Scientific, USA). The PCR products were separated on 1.5% agarose gel at 5V/cm, and the bands were visualized under UV light; 700 bp bands were excised and eluted using a gel purification kit (Illustra GFX 96 PCR Purification kit, GE Healthcare, USA). The purified DNA was sequenced at the Genomic Facility Laboratory at University of Guelph. Bacterial strains were identified based on 16S rDNA sequence comparisons using BLAST searches to Genbank.

### D. Microscopic Mechanisms of Action

Microscope slides were used to view the in vitro interactions between pathogen and M6 endophyte. The slide was coated with a thin layer of PDA, then 50 μl of bacterial endophyte (culture grown overnight in LB incubated at 37° C.) was applied adjacent to 50 μl of F. graminearum (mycelia grown for 24-48 h in potato dextrose broth at 25° C., 100 rpm). The slide was incubated at 25° C. for 24 h then stained with neutral red (Cat. #57993Sigma Aldrich, USA) or Evans blue (Cat. # E2129, Sigma Aldrich, USA) by placing 100 μl of stain on the slide, followed by a 3-5 min incubation at room temperature, then washing 3-4 times with deionized water. Pictures were taken using a light microscope (MZ8, Leica, Wetzlar, Germany). There were 3-4 replicates for each slide.

### E. Testing in Planta Activity of M6 to Suppress Gibberella Ear Rot in Corn (2 Replicates)

The candidate endophyte M6 was used to test for suppression of Gibberella ear rot disease caused by F. graminearum in maize plants in a greenhouse (Summer, 2012). Seeds of a susceptible Ontario maize hybrid (35F40, obtained from Prof. A. Schaafsma and Dr. V. Limay Rios, Ridgetown College) were coated with endophytic innoculant. To prepare endophytic bacterial inoculant, bacteria were grown for 24 hr at 37° C. in liquid PDA media, centrifuged, washed and suspended in PBS buffer to OD600 of 0.5. Thereafter, 500 μl of each bacterial suspension were mixed with 10 ml polyvinyl pyrrolidine (Cat. #9003-39-8, Sigma Aldrich, USA) as a seed-coating agent; the mixture was incubated for 2 hr on a horizontal shaker (Adjustable Tilt Rocker, National labnet company, Mandel Scientific). Coated seeds were germinated over wet paper towels in Petri dishes in the dark for 7 days; uniformly sized seedlings were then transferred into pots containing Turface clay in the greenhouse under the following growth conditions: (28° C./20° C., 16 h:8 h, ≧800 μmol m-2 s-1 at pot level, with high pressure sodium and metal halide lamps with GroLux bulbs) using drip irrigation with modified Hoagland's solution until maturity (Gaudin et al., 2011). For each inoculant treatment or control, there was 20 plants/treatment arranged randomly. One ml of F. graminearum spore suspension (20, 000 spores/ml, supplied Dr. V. Limay Rios, Ridgetown College) was applied twice at 3 day intervals beginning at the silking stage. To ensure high titre of the endophyte, the endophyte was also sprayed simultaneously with the pathogen inoculation. Positive control was seeds coated only with PVP followed by fungicide spraying (PROLINE® 480 SC Foliar Fungicide, Bayer crop science) at the post-silking stage prior to infection with the fungal pathogen. Negative control was seed coated only with PVP then challenged with Fusarium spraying only. At full maturity stage, ears were scored for percent of apparent infection, which is the length of diseased area from the top “infection site” relative to the total length of the ear. The other phenotype measured was total kernel weight at harvesting. The entire experiment was repeated in summer 2013. Results were analyzed and compared by Mann-Whitney t-test (P<0.05).

### F. Testing in Planta Activity of M6 to Suppress Fusarium Head Blight in Wheat (2 Replicates)

The candidate endophyte M6 was used to test for suppression of Fusarium head blight disease caused by F. graminearum in Wheat plants in a greenhouse in a two independent replicates (Summer, 2013). Seeds of a spring wheat (cultivar Quantum, obtained from Prof. A. Schaafsma and Dr. V. Limay Rios, Ridgetown College, Canada) were coated with endophytic innoculant prepared as described above. Coated seeds were germinated over wet paper towels in Petri dishes in the dark for 7 days; uniformly sized seedlings were then transferred into pots containing Turface clay in the greenhouse under the following growth conditions: (28° C./20° C., 16 h:8 h, ≧800 μmol m-2 s-1 at pot level, with high pressure sodium and metal halide lamps with GroLux bulbs) using drip irrigation with modified Hoagland's solution until maturity (Gaudin et al., 2011). For each innoculant treatment or control, there was 20 plants/treatment arranged randomly. One ml of F. graminearum spore suspension (20, 000 spores/ml, supplied Dr. V. Limay Rios, Ridgetown College) was applied twice at 3 day intervals beginning at the silking stage. To ensure high titre of the endophyte, the endophyte was also sprayed simultaneously with the pathogen inoculation. Positive control was seeds coated only with PVP followed by fungicide spraying (PROLINE® 480 SC Foliar Fungicide, Bayer crop science) at the fruiting stage prior to infection with the fungal pathogen. Negative control was seed coated only with PVP then challenged with Fusarium spraying only. At full maturity stage, plants were scored for percent of infected seeds relative to total number of seeds in each plant. The other phenotype measured was yield of individual plants at harvesting. Results were analyzed and compared by Mann-Whitney t-test (P<0.05).

### Example 2

### A. Identifying the Genes Responsible for the Antifungal Activity Using the Transposon Mutagenesis Technique

Tn5 transposon mutagenesis was conducting on M6 using the EZ-Tn5<R6Kyori/KAN-2>Tnp Transposome from Epicentre (FIG. 6 (A-C)). From 4800 knockout mutants tested, ten mutants caused loss of anti F. graminearum activity (Table 1).

### B. In Planta Validation of Candidate Genes

The in planta validation of five of the candidate genes was tested in the greenhouse in two independent replicates. The mutants or wild endophytes were applied as spray only for half of the plants and as seed coat only for the second half to assess the best method for efficient application. In the greenhouse trial 1, three mutants failed to protect the plant from F. graminearum. In the greenhouse trial 2, all the five mutants tested failed to protect the plant from F. graminearum as shown in FIGS. 7 and 8.

### Materials and Methods

### A. Identifying the Genes Responsible for the Antifungal Activity Using Transposon Mutagenesis Technique

### Step 1. Preparation of Competent Cells for a Candidate Bacterial Endophyte

One liter of LB broth was inoculated with 10 ml bacterial culture grown overnight at (37° C. at 250 rpm) until early Log phase (OD600=0.4-0.6). The cells were harvested by chilling for 15 minutes on ice, and then centrifuged in a 4° C. rotor at 4000×g for 15 minutes. The cells were then resuspended in 1 L of 4° C. cold water, centrifuged, resuspended in 0.5 L cold water, centrifuged, re-suspended in 20 mL of cold 10% glycerol, recentrifuged and the pellet re-suspended tin 3 mL of 10% glycerol from which 40 ul aliquots were made and frozen at −80° C.

### Step 2. Transposon Mutagenesis

Tn5 transposon mutagenesis was conducting using the EZ-Tn5<R6Kyori/KAN-2>Tnp Transposome from Epicentre, which has reportedly achieved success for both Gram negative and Gram positive strains. M6 bacterial endophyte strain was transformed using electroporation, using 40 μl competent cells with 1 μl of the EZ-Tn5<R6Kyori/KAN-2>Tnp Transposome as described by the manufacturer. This transposome is a KanR linear vector pre-packaged with transposase protein with an E. coli origin of replication, hence any transformed colonies that gain kanamycin resistance are likely to contain insertions in genomic DNA. The electroporated cells were immediately recovered by adding fresh LB media to 1 ml final volume with gentle mixing by pipetting then incubated at 37° C., 250 rpm for one hour to allow protein expression. 100 μl of cells were plated on solid LB media containing 35 μg/ml kanamycin. Endophytes M6 was previously pre-checked for susceptibility to kanamycin.

### Step 3. Mutant Screening

In this study, 4800 KanR knock out mutants were screened (predicted to contain genomic DNA insertions of the transposon) for loss or gain of antifungal activity using the agar diffusion method as described above. Specifically, only knock out mutants that showed dramatic loss or expansion of the radius of the zone of inhibition of growth of F. graminearum on agar in vitro were scored. Candidate clones were rescreened for phenotype confirmation prior to gene rescue.

### Step 4. Rescue of the Disrupted Gene and Identification

Genomic DNA containing the transposon insertion in E. coli on Kanamycin media was rescued, taking advantage of the E. coli origin of replication within the Tn5 transposon vector and gene encoding KanR, according to the manufacturer's instructions (Epicentre). Briefly, genomic DNA was isolated from candidate mutants, restricted with an enzyme that cuts outside of the transposon vector, then resulting genomic fragments were self-ligated resulting in the genomic fragment containing the transposon becoming a KanR plasmid following subsequent electroporation into E. coli and plating of transformed clones onto kanamycin media. Plasmids containing the Tn5 transposon were sequenced using a transposon-specific read-out primer to identify the open reading frame of the disrupted gene. The candidate genes (operon) responsible for the anti-fungal activity were identified by BLAST homology searching. Ten potential candidate genes were identified in this study.

### B. In Planta Validation of Candidate Genes

The in planta importance of five of the candidate gene was tested in a greenhouse in two independent replicates in which corn plants were inoculated with the wild type and mutant endophyte side-by-side, followed by infection with F. graminearum following the same protocol as the initial greenhouse trials except for mutant or wild endophytes were applied as spray only for half of the plants and as seed coat only for the second half to assess the best method for efficient application.

### C. Statistical Analyses

All statistical analysis was performed using Prism Software version 5 (Graphpad Software, USA). All error bars shown represent the range of data points.

### Example 3

### Transcriptional Analysis in the Presence and Absence of the Pathogen

To test if the candidate genes are inducible by F. graminearum or constitutively expressed, Real-time PCR analysis was conducted using gene specific primers shown in Table 2.

4 ml of endophyte M6 grown overnight were added to 400 ml LB to OD600=0.021 then incubated at 37 with shaking rpm=250 until OD600=0.14 then divided into three 250 ml sterilized flasks each contains 100 ml of the endophyte culture:

1—M6+3 ml YPD media
2—M6+3 ml of 48 hr F. graminearum grown in YPD media incubated at room temperature with slow shaking (rpm=50)
3—M6+3 ml YPD media+0.1 g chitin (company and catalogue number)

Samples were taken from the cultures for RNA extraction after 1, 2, 3 and 4 hours, the OD600 of the cultures at these times were 0.79, 1.14, 1.41 and 1.51.

RNA was extracted following a standard protocol (E.Z.N.A Total RNA kit, catalogue #R6834-01, Omega Bio-tek, USA). To remove any DNA contamination from the extracted RNA, samples were treated with DNase (Promega) following the manufacture's protocol.

TABLE 2
Gene-specific primers used in
Real-time PCR gene expression analysis.

| Mutant ID | Real-time PCR primers |
|---|---|
| m1B3* | B3f 5′-ACCCCGGTCATCTCTGATA |
| | ATG-3′ (SEQ ID NO: 24) |
| | B3r 5′-GCGCGCTGTCTCTTTCATC-3′ |
| | (SEQ ID NO: 25) |
| m1C5* | C5f 5′-GGCGCGCGTTATCATTGTA-3′ |
| | (SEQ ID NO: 26) |
| | C5r 5′-CGCTGCGCACCAACTTTA-3′ |
| | (SEQ ID NO: 27) |
| m2D7 | 2D7f 5′-GTGCTGAAGCGATCTTAG |
| | GG-3′ (SEQ ID NO: 28) |
| | 2D7r 5′-GTTCCATCAGGCTTTTTC |
| | CA-3′ (SEQ ID NO: 29) |
| m5D7* | 5D7f 5′-GGCATAACTTCCTGCGCT |
| | AC-3′ (SEQ ID NO: 30) |
| | 5D7r 5′-CAGTACGCCATCAATCAT |
| | CG-3′ (SEQ ID NO: 31) |
| m9F12* | F12f 5′-CGCTTCGCGATGCTGAAT-3′ |
| | (SEQ ID NO: 32) |
| | F12r 5′-GGCCGAACGCCTTCTCA-3′ |
| | (SEQ ID NO: 33) |
| m4B9 | B9f 5′-TGTTTTATGCTTAAACTGG |
| | CGATT-3′ (SEQ ID NO: 34) |
| | B9r 5′-CGAATGCGGTGGGATATCA-3′ |
| | (SEQ ID NO: 35) |
| m15A12 | A12f 5′-GACGATGCGACCGGTTTT-3′ |
| | (SEQ ID NO: 36) |
| | A12r 5′-AGCCTATCGACCGGATGC |
| | T-3′ (SEQ ID NO: 37) |
| m1H3 | H3f 5′-TGGCGGGAGTCCATCGT-3′ |
| | (SEQ ID NO: 38) |
| | H3r 5′-TGTTCAAGGAGACGCAGCA |
| | T-3′ (SEQ ID NO: 39) |
| m3H2 | H2f 5′-CCGACCTGGACCTCCAAAA-3′ |
| | (SEQ ID NO: 40) |
| | H2r 5′-GCCGCGGTTGTTAACGATA-3′ |
| | (SEQ ID NO: 41) |
| m7D5 | D5f 5′-GGGGACAGTAACGACGAAA |
| | C-3′ (SEQ ID NO: 42) |
| | D52r 5′-CGGCAATCTGTCGATATG |
| | AA-3′ (SEQ ID NO: 43) |
| m10A8 | A8f 5′-GGAGTCAAAACACGGAATT |
| | TACG-3′ (SEQ ID NO: 44) |
| | A8r 5′-ATCTGATAAGCAGGGAAGA |
| | TCTCTTT-3′ (SEQ ID NO: 45) |
| m5B1 | B1f 5′-GCAGTTTGACGCCCTTAAT |
| | GTT-3′ (SEQ ID NO: 46) |
| | B1r 5′-GGCGCAGGAGAGTGCAA-3′ |
| | (SEQ ID NO: 47) |
| m8E1 | E1f 5′-TCAAACGTTCAGCATTGAG |
| | C-3′ (SEQ ID NO: 48) |
| | E1r 5′-TATGCCGGATTAACGACCT |
| | C-3′ (SEQ ID NO: 49) |
| Fusaric | FusEf 5′-GGGGACAGTAACGACGA |
| acid | AAC-3′ (SEQ ID NO: 50) |
| resistance | FusEr 5′-CGGCAATCTGTCGATAT |
| protein | GAA-3′ (SEQ ID NO: 51) |
| gene | |
| Phenazine | Phzf 5′-TACGTTGAAGCCCGTAAA |
| F | GG-3′ (SEQ ID NO: 52) |
| gene | Phzr 5′-AGAAAAAGCGGCTGACAA |
| | AA-3′ (SEQ ID NO: 53) |

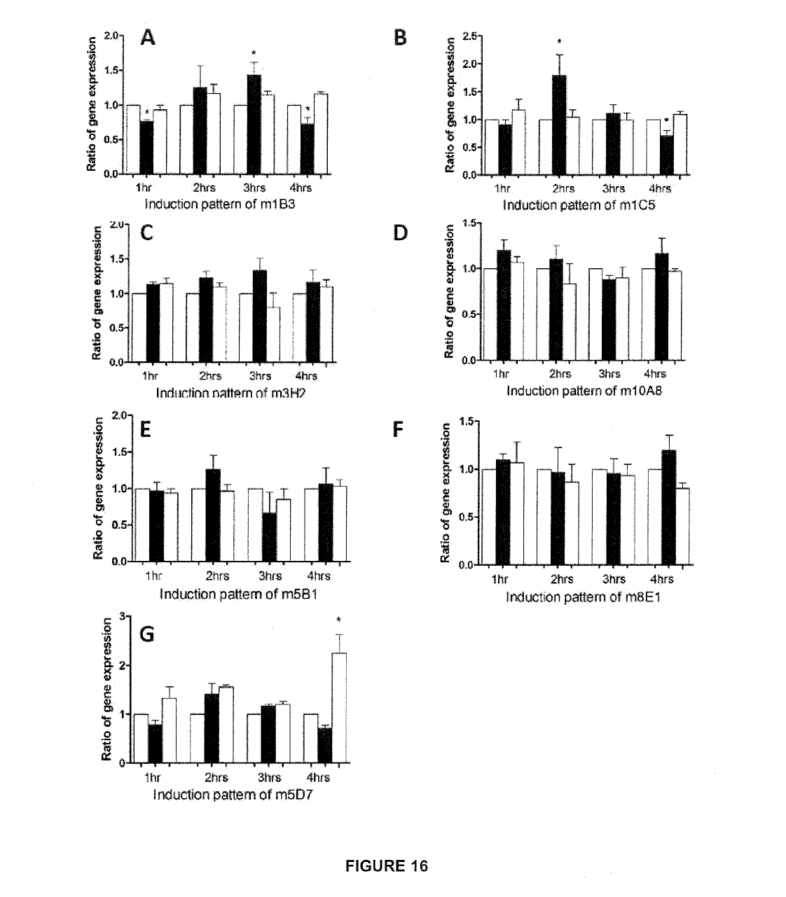

A number of the genes including m2D7, m9F12, m4B9, m15A12, m1H3 and m7D5 were significantly inducible by Fusarium (FIG. 14) while a number of additional genes were minimally inducible by Fusarium (FIG. 16).

### Example 4

### GFP-Tagging for Ecology Study

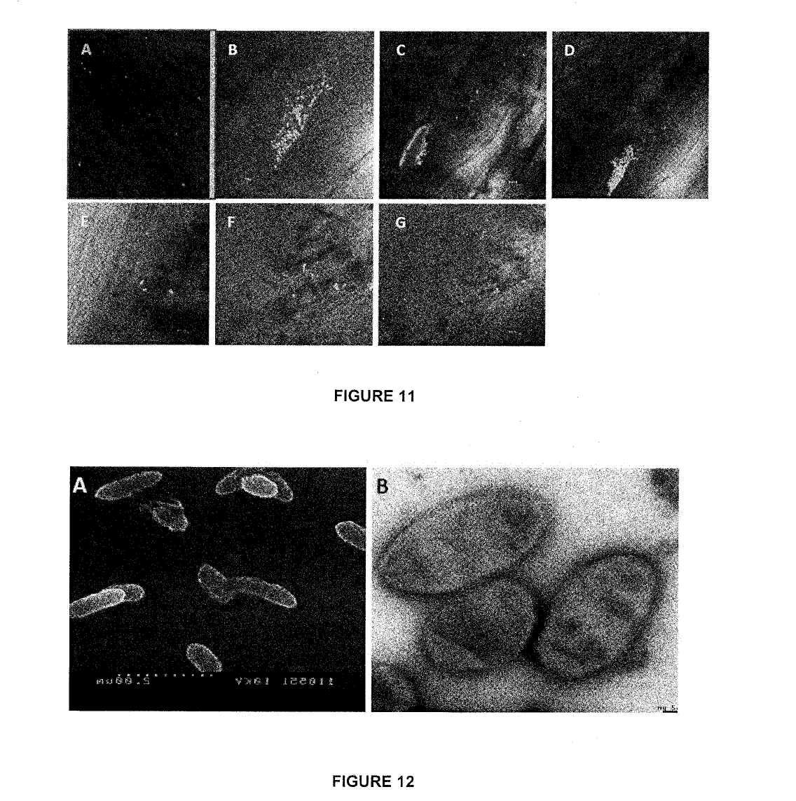

Competent cells were prepared as described above. Then M6 was transformed by GFP-plasmid (pDSK-GFPuv) [20]. The plasmid carrying gfp gene was extracted from GFP-Ecoli following standard protocol (Quantum prep. #732-6100, Bio-Rad, USA) then introduced to bacterial cells by electroporation [21]. Briefly, suspensions of 40 μl of cold competent M6 cells were mixed with 3 μl of plasmid DNA (100 ng/μl) then electroporated at 1.6 KV for 1 s using Bio-Rad Gene Pulser 200/2.0 (Bio-Rad Hercules, USA). After electroporation, cells were incubated for 1 h in 1 ml of LB at 37° C. with shaking. Transformed cells were plated on LB agar containing Kanamycin (35 μg/μl) and incubated for 24 h at 37° C. then the plate was examined for fluorescent colonies using (Illumatool, Montreal Biotech Inc., Canada). Corn and millets seeds were surface as described above and coated with GFP-tagged M6 and then planted first on wetted paper towels for one week then transferred to the greenhouse. Plants were screened by confocal laser scanning microscope at microscopy imaging facility, advanced analysis centre at the University of Guelph (FIG. 11).

### Example 5

### Total Genome Sequencing

DNA was precipitated by adding 1/10 volume of 0.3 sodium acetate, pH 5.2 followed by the addition of 2.5 volumes cold 100% ethanol then kept at −20 for 20 min. The DNA was then centrifuged for 15 min at max speed (Centrifuge 5415D, Eppendorf), supernatant was decanted and the ppt. was re-suspended in 1 ml of 70% ethanol, centrifuged for 10 min at max speed, the supernatant was carefully decanted and the DNA was left to be air-dried.

### Example 6

### Electron Microscopy of M6 Bacteria

Scanning and transmission electron microscopy imaging was conducted to visualize the external appearance of the candidate bacterium following standard protocols as follows.

For scanning electron microscopy; bacterial culture was plated on LB plates, incubated overnight (37° C., without shaking), then scratched and suspended in phosphate buffer (pH 7). Around 2 μl of the suspension was placed on a carbon disc and allowed to dry (one hr). The bacteria was washed with phosphate buffer, fixed with 2% glutaraldehyde (one hr), treated with 1% osmium tetroxide (30 min), then gradually dehydrated using an ethanol series (50%, 70%, 80%, 90% and 100%). The dried bacterial film was coated with gold and examined by SEM (Hitachi S-570 SEM, Hitachi High Technologies, Tokyo, Japan) at the Imaging Facility, Department of Food Science, University of Guelph.

For transmission electron microscopy; bacterial culture was grown overnight in LB medium (37° C., 225 rpm). Thereafter, 5 μl of the culture was pipetted onto a 200 mesh copper grid coated with formvar and carbon. The excess fluid was removed onto a filter and the grid stained for 10 sec with 2% uranyl acetate. The bacterial sample was examined by TEM (FEI Tecnai F20 G2 operating at 200 Kv, Gatan 4K CCD camera) at the Imaging Facility, College of Biological Sciences, University of Guelph.

As shown in FIG. 12, the M6 bacteria exhibit a rod-like shape typical of Enterobacter.

### Example 7

### Suppression of DON Production During Storage

ELISA analysis was conducted to test the accumulation of DON in corn and wheat seeds during storage. Seeds were ground for 40 sec using M2 Stein mill (Fred Stein Lab, Inc. Atchinson, Kans., USA). Ground samples (5 g) were diluted in distilled water (1:5 w/v), shaked vigorously for 3 min using a bench top reciprocal Eberbach shaker (Eberbach Corp, Ann Arbor, Mich.). Thereafter, 2 ml aliquot of the suspension were centrifuged (8000 rpm for 60 sec) and diluted in distilled water when necessary. ELISA analysis was carried out with the EZ-Tox™ DON Test (Diagnostix Ltd., Mississauga, ON, Canada) following the manufacturer's protocol. There were three replicates for each treatment. Results were analyzed and compared by the Mann-Whitney t-test (P<0.05).

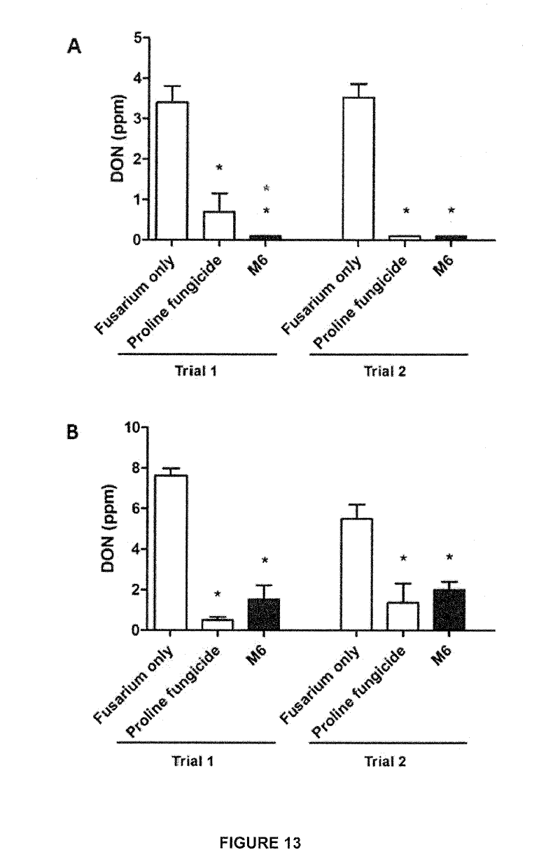

As shown in FIG. 13, a significant reduction in DON mycotoxin accumulation was observed during storage following treatment with the M6 bacterial endopohyte in both corn and wheat seeds.

### Example 8

Bio-Guided Fractionation Combined with LC-MS Analysis to Detect Candidate Antifungal Compounds

In order to detect the presence of potential antifungal compounds as predicted by the candidate genes, Bio-guided fractionation combined with LC-MS analysis was conducted. The candidate endophyte M6 was grown for 48 h on Katznelson and Lochhead liquid medium (Paulus and Gray, J. Biol. Chem. 239, 865 (1964)), harvested by freeze drying, then the lyophilized powder from each strain was extracted by methanol. The methanolic extracts were run on a Luna C18 column with a gradient of 0.1% formic acid and 0.1% formic in acetonitrile. Peaks were analyzed by mass spectroscopy (Agilent 6340 Ion Trap), ESI, positive ion mode. LC-Mass analysis was conducted at the Mass Spectroscopy Facility, McMaster University, Ontario, Canada.

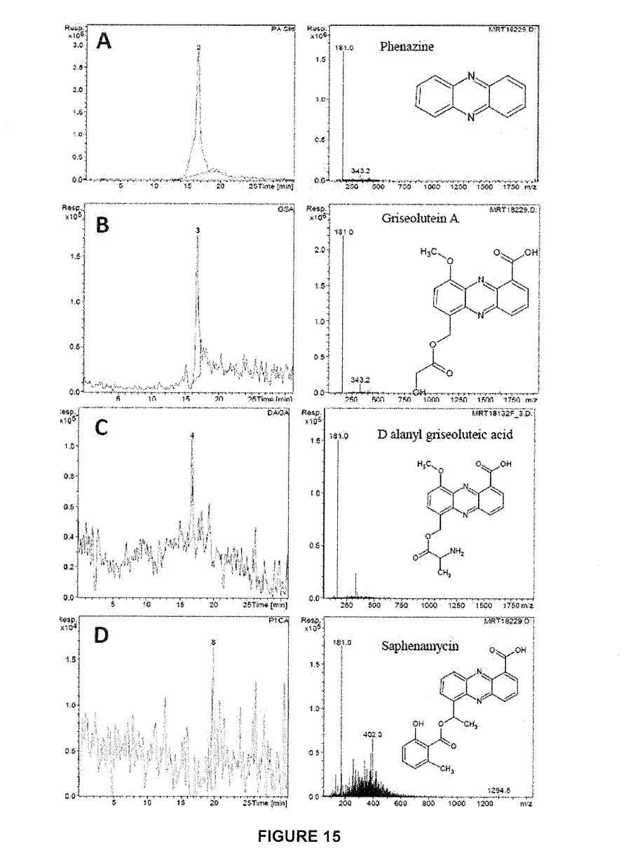

As shown in FIG. 15, compounds detected in the active anti-Fusarium broth include a number of phenazine derivatives.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents, and patent applications are herein incorporated by reference in their entirety to the same extent as if each individual publication, patent or patent application was specifically and individually indicated to be incorporated by reference in its entirety.

### Sequences

16S rDNA Sequence
SEQ ID NO: 1
TAGCTAACGCGTTAAGTCGACCGCCTGGGGAGTACGGCCGCAAGGTTAAAACTCAAATGA
ATTGACGGGGGCCCGCACAAGCGGTGGAGCATGTGGTTTAATTCGATGCAACGCGAAGAA
CCTTACCTACTCTTGACATCCAGAGAACTTANCAGAGATGNNTTGGTGCCTTCGGGAACT
CTGAGACAGGTGCTGCATGGCTGTCGTCAGCTCGTGTTGTGAAATGTTGGGTTAAGTCCC
GCAACGAGCGCAACCCTTATCCTTTGTTGCCAGCGGTNNGGCCGGGAACTCAAAGGAGAC
TGCCAGTGATAAACTGGAGGAAGGTGGGGATGACGTCAAGTCATCATGGCCCTTACGAGT
AGGGCTACACACGTGCTACAATGGCGCATACAAAGAGAAGCGACGACCTCATAAAGTGCG
TCGTAGTCCGGATTGGAGTCTGCAACTCGACTCCATACGGTGAATACGTTCCCGGGCCTT
GTACACACCGCCCGTCACACCATGGGAGTGGGTTGCAAAAGAAGTAGGTAGCTTAACCTT
CGGGAGGGCGCTTACCACTTTGTGATTCATGACTGGGGTGAAGTCGTAACNAAGGT
Gene ID: m1B3
SEQ ID NO: 2
GTGAATACCATTGGCATAAACAGCGAGCCCATCCTGACGCACAGTGGCTTTAGCATTACC
GCCGATACCACTCTTGCCGCAGACAGGCACTATGACGTTATCTATCTTCCTGCCCTGTGG
CGCAATCCTCGTGCAGTGGTCAGACAACAGCCTGAACTCCTGGCATGGCTTAGCGAACAG
GCGGCGCGAGGGACCCGCATCGCGGCCGTCGGAACGGGCTGCTGTTTCCTGGCGGAATCG
GGATTGCTCAACGGGAAACCCGCCACCACCCACTGGCACTACTTCAAACAGTTCTCGCGT
GACTACCCCAACGTAAAATTACAAACCAAACATTTTCTCACGCAGGCCGATAATATTTAC
TGTGCCGCCAGCGTCAAAGCCCTCTCAGATCTGACCATCCATTTCATCGAAACGATATAC
GGGAAACGTGTAGCCACACATACCCAACGGACCTTTTTCCATGAAATTCGGAGTCAGTTT
GATCGCCAGTGTTACAGTGAAGAAAACAAACCCCATCCGGATGAAGATATTGTTCAAATT
CAAATCTGGATAAAAGCCAACTGCGCCTCGGATATATCCATGCAAAATCTTGCCGATATG
GCTGGCATGAGTTTGCGCAACTTTAATCGCCGCTTTAAAAATGCCACCGATATATCCCCC
CTGCAGTATTTATTAACCGCCAGAATTGAATCCGCCATGACAATGCTGCAATCCACCAAT
CTGAGCATTCAGGAGATTGCGAATGCGGTGGGATATCAGGATATTGCGCACTTTAATCGC
CAGTTTAAGCATAAAACAACGGTTTCACCGGGGGATTACCGTAAGACCGTCCGGGCAAAG
ATGTTTAGTGCATAA
Gene ID: m9F12
SEQ ID NO: 3
ATGCCCGCAAAATCATCAGGAAGCGCGTGGGAGCGTTTTGCCGGAGTATTACGTAATGCG
CAAACGGAATGTATTGTTACGACAGCAAAGGGAGCAGAAACGCTAGGCCAGCTATCACTT
CCGTTATCCCCGCTTATTTTTACCTTCGACAAACCAGACACAGCTGCGCTCCCTGCGGGC
TATCGTCTGCATCCTCTTGATCGCACGTTCTCCGGGGCATTTCACCCCGTTCCGGTAGCG
GAAAACGATCTGGCTTTTTTACAGTACACCTCGGGTTCGACGGGCTCTCCGAAGGGCGTG
ATGGTGACCCATGGCAACCTGTGGGCCAACTCGCATGCCATTCACCGCTTTTTCGGCCAT
CACAGCGAAAGCCGGGGCACGATCTGGCTGCCGCATTTTCATGATATGGGGCTGATTGGC
GGGCTACTGCAGCCCGTGTTTGGCGCATTCCCCTGTCGGGTGATGTCGCCCATGATGTTA
ATGAAAAATCCCCTTAACTGGCTCAAACATATTTCTGACTATCAGGCGACGACCTCCGGC
GGCCCTAATTTCGCCTACGATCTGTGCGTGCGCAAGATTGGCAGAGAGCAAGTTGAGGCA
TTAGATCTTTCTCGCTGGGATGTGGCTTTTTGCGGCGCAGAGCCGATTCGGCCCGCCACG
CTACAGCAGTTTAGCGAACACTTTGCGCCCGCAGGGTTTCGGCCCGGCGCGTTCCTGCCC
TGCTACGGCATGGCGGAAACCACGCTCATCGTCACCGGGATGGAGAAAGGACAAGGGCTC
CGCGTTTCCGACGAGGCCGGTACGGTGAGCTGCGGGCAGGCTCTGCCGGATACCGAGGTG
CGTATCGTCGATCCCGATCGCCATCAGCCGCTTGCTGATGGTGAGAGCGGGGAAATATGG
CTGCGTGGTCCGAGCGTTGCCGCAGGATACTGGGACAATGACGCCGCCACACGCGAAACC
TTCCAGGCGTCTCTGGCTGGCCATCCGCACCCGTGGCTGCGCAGCGGCGATATGGGTTTT
CTCCAGTCCGGCCATTTGTACGTCACCGGACGACTCAAAGAGCTGCTGATTATCAACGGT
CAGAACCACTACCCGACGGATATTGAAGAGACGATCCGCCAGGCCGATCCCGCGCTGGCG
GAAGCGACGGTTTGCGTGTTTGCCAGTGAAGACGAGCGCCCGGTTGCGCTGCTTGAGCTG
ATGACGCGCCATAAAAACGATCTCGATATGGCGACGCTGGCACCGTCCGTGACCGCGGCG
GTGGCGGAGCGGCACGGCATCACGCTGGATGAACTGCTCCTTGTCGGGCGAAGGGCAATT
CCCCGCACCACCAGCGGGAAACTACAGCGCACCCGCGCGAAAGCGATGCACCAGCAGGGA
ACCCTGGAAGTAGCCTGGCGCAGCTGCCAGGACGCGTCGAAACCTGTTGAACTCGCGGGG
GAAACCCCACCCGCGCTGGCGGCGCTGATAGCCGGGATAATCAGCAGCGCGATGAACACG
ACGATCGGCGAATCCCAGTGGGACGAGGCGTTTACCGGCTTTGGCATGAGCTCTCTGCAG
GCGGTGGGCGTGATTGGCGAGCTTGAACAGCGGCTGGGCCGCGAGCTCTCTCCCGCGCTG
ATTTATGACTACCCCACCATCAATCGGCTGGCGGCCGCGCTGGGGCAACCCGCTGCGGCC
CGGCCGGTCAGCTCAGCCGTCGCGGAGAGCGCCATTGCGGTGATTGGCATCGGCGTGGAG
CTGCCGGGACATAGCGGCGTGGAGGCGCTGTGGTCGCTGCTGCAGCAGGGCCACAGCACG
ACCGGCGAGATCCCGGCGCACCGCTGGCGTACCTCGTCCCTTGACGGTTTTAACCGTAAA
GGCAGTTTCTTCGACGAGGTCGACGCGTTCGACGCAGGCTACTTCGGCATCTCTCCCCGT
GAGGCCGTCTATATCGATCCGCAGCATCGTCTGCTGTTAGAAACCGTTCAACAGGCGCTA
ACCGATGCCGGCCTTAAGGCGTCCTCCCTGCGCGGTAGCGATACGGCGGTCTTTGTTGGG
ATCAGCGCCAGCGACTACGCGCTGGCCTGCGGCGATAACGTCTCGGCCTACAGCGGCTTA
GGCAACGCGCACAGTATCGCGGCCAACCGAATTTCTTATCTTTATGATTTAAAAGGTCCA
AGCGTCGCCGTCGACACGGCCTGTTCTTCCTCGCTGGTGGCGATAGAGGGGGCAATGCAG
AGCCTGCGGGCCGGACGTTGCGCTCTGGCCATTGCCGGAGGCGTTAATCTGGCGCTGACG
CCACATTTGCAAAAAGTCTTCACCGAAGCCCAGATGCTGGCCCCCGACGGCCGGTGTAAA
ACCTTCGACGCCCGCGCGGATGGCTATGTTCGTGGCGAAGGGTGTGGCGTCGTGGTGCTT
AAGCCGCTTTCACAGGCGCTGGCGGATGGCGATCGGGTTTATGCCACGCTGGTGGCGAGC
GCCGTGAATCAGGACGGCCGCAGCAACGGCATTACCGCGCCAAATGGCCCATCGCAGCAG
GCGGTCATCCTGCAGGCGATGGCGGACGCCGGGTTGGACAGCGACAGCATTGACTATATC
GAAGCGCACGGTACGGGAACCGCGCTTGGCGATCTGATTGAATATCAGGCGCTGGAAGCG
GTGTTTGCGGACCGGAAAAAGACCGCACCTGTCCAGGTGGGTTCGATCAAAACCAACATT
GGCCACCTTGAGGCGGCGGCGGGCGTGCTGGGCGTGGTGAAAACGTCTCTGATGCTGCAC
TTCCGGCAATACGTACCTCACCTCAATTTTCAGCAGAAAAACCCGCATATTGCGGCGATT
AGCCGTCATGTTGAGGTGAGCGGCGCGCAGCCTGCCTCATGGCATGCCGATGGCGAAGCG
CGCTATGCGGGCGTAAGCAGCTTTGGCTTCGGCGGTACCAACGGTCATGTGATTTTGCGC
AGCGCGCCAGCGGTGGAAAAACGCCAGGAGCCCGCTGCGCCGCACGGCCTGCTTCTGGTC
GGTTCACATGATAAAGGGGCGTTTACCCTTCAGCGGGAGGCGGTCAAAAAAGGGTTATCG
ACGTGCCAGGAGAGCGATATTGCCACCTGGTGTCGGCTGGTGAACACCCGCTACGACGCG
GCCCGCTATCGCGGCGTGGCGTATGGCGCGGATCGCTCCCAGCTGGCGGAAAGCCTTGCG
CAGCTCACCGTCTGCAAGGTGGGTAAAGCCCAGCCCCAGGTCTGGCTCTTCCCGGGGCAG
GGCACCCAGCAAATCGGCATGGGTGCCGAGCTGTATCACCATCTGCCGCACTATCGCACC
CAGTTTGACGCGCTGGCGACGACTATTCAGCAGCGCTATCAGATTGATATTACGCAGGCG
CTGTTTGCCCGTGACGACAGCTGGCAGCGCTGCGCCAGAACGTGTCAGCTCTCATTATTT
GCCTGTAGCTACGCGCTTGCTCAGAGCGTGATGCAGTTCGGCCCGCGTCCGGCTGCCGTA
ATGGGGCACAGCCTGGGAGAGTACTGCGCGGCGGTTATCGCTGGCTATCTCTCGCTGGAC
GACGGGCTGGCAATGGTTCATCAGCGCGCGCTGTTGATGTCAGCCCTGACGCAGGAAGGG
GCGATGGCTGTCGTCTTCAGCGGCGAAGCCGACGTCCGTCAGATGATTTCCCCCTGGACG
GGCGACATTGATATTGCCGCATTCAACACGCCGACATTGACCACCATCGCAGGCAGTCGG
GCGGCGATTGACGCCTGCCTTCAGGCCATATCTTCAAAAGGCGGTCACGCCAGAAAAATT
AAAACTGCCAGCGCATTTCACTCCTCGATGATGGATCCGATCCTCGGCGCCTGGCGCGAG
TGGCTGGTCAACAACGTCACCTTCACCCGCGGGACGATCCCGTTTTACAGCAACCTGAAC
GGTGAGGCGTGCGACCGCACCGACGCCGACTACTGGACCCGGCAAATTCGCCAGCCCGTG
AGTTTCCTTCAGGGCGTGCAAAACGTGCTGGCACAGGGTGAGTTCACCTTTATCGATCTG
AGCGCGGACGGTTCGCTGGGCAAATTTGTGACCGCAACTGACCGCCGTCACCGGGTGCTG
GCCGCAGGCGACCGGCGACATGAGTACAAATCACTGCTGACGCTGCTGGGTACGCTGTGG
CAGCAAGGGCACGACATCAACTGGAGCGGGCTGTACCACGCGACCACGCGGGAGGCGCTA
ACCCTGCCCGCCATCCAGTTCTGCCGCAAACGTTACTGGCTGGCGGGTGAGACGCCAGCG
CAGACCCCATCTGCAAAAGAGGACGCTATGTCAAATCAACACCATTTAGCCGCTGAAATA
AAAGCGATTATTGCCGGTTTTCTTGAGGCGGATCCCGCCGCGCTTGACGACTCTCTGCCG
TTCCTGGAAATGGGGGCGGACTCGCTGGTGCTGCTGGATGCCATCAATACCATTAAAGAC
CGCTTTGGCGTAGCCATCCCGGTGCGGGCGCTGTTTGAAGAGCTCAATACGCTGGACGCG
GTGATCGGATATGTGGTGGAGCACGCGCAGCCGGCGGCTTCGCTCACCACCCCGGAAACC
GCCGGCCTGGCGGCACAGCCTGTCGCGGCACCGCAGGGTACCAGCAGGCCGGTGCCTGAT
ACGGTTCAGGATCTGATTGCCCGCCAGCTGGAGCTCATGTCCCAGCAGCTAAATTTGCTT
AACGGCACGGCGCAGGCTCTCCCGATGCCAGCCGCACCCGCGACGCCGGACGTTATCGCG
CCTGCGCCCGTCGTGGCCCCGACCGCACCGGTGAAGGCCAGCGCGCACAACAGCTGGTTT
AAAAAAGAGACCAAAAAGGTCTCCCTCGGCGCTGAGCGCGACCAGCATCTGGCGCAACTC
ACCGAACGGTTTGTCGATAAAACGGGCGGGTCAAAACGCAATGCCCAGCAATACCGCGCC
GTGCTCGCGGATAACCGGGCCTCTGCGGGCTTTCGTTTATCCACCAAAGAGATGCTTTAC
CCCTTAGTGGGAGAGCGCTCTCAGGGTTCGCGCATCTGGGATGTCGATGGCAACGAGTAT
ATTGATTTTACGATGGGGTTTGGCGCTAACCTGCTGGGACATGCGCCGGACTGCGTGCAG
CAGGCCGTTGCCGACCAGCTGGCGCGGGGCATGCAGATTGGTCCGCAAAGCGCCCTGGCG
GGCGAGGTGGCAACGCTTATCAGCGAGCTGACCGGCCAGCAGCGCGTGGCATTTTGTAAC
TCCGGCTCCGAAGCGGTAATGAGCGCCGTGCGCCTGGCGCGGGCCGTGACGGGGAAAAAT
AAAGTCGCGCTGTTTAGCGGCTCGTATCATGGCGTGTTTGACGGCATTCTGGGGCGACAG
CAGGGGGGAGAAACCCCCGAGCGCGCGACGCCGATTGCCGCAGGCACGCCGCCATCGCTG
GTGGACGACCTGCTGGTGCTCGACTACGGCAGCGAAGAGAGCCTGGCGCTTATCGCCCGC
TATGCCGCCGAGTTGGCGGTGGTGATCGTCGAACCGGTGCAGAGCCGTTATCCCGATCAT
CAGCCACGCGACTATCTGCATACCCTGCGTGAACTGACGACAACCCACAACATTGCGCTG
ATGTTTGACGAGGTGATCACCGGTTTCCGTCTGGCTGCCGGCGGCGCGCAGGCGTATTAC
GGCGTTCAGGCGGATATCGCCTCCTACGGAAAGATTGTCGGCGGCGGTATGCCGATCGGC
GTGATTGCAGGCTCTGCGCGCTTTATGGACAGTATCGACGGCGGATTCTGGCAGTATGGC
GATGACTCCTGGCCGCAGGCTGAACTGATCTTCTTTGCCGGTACGTTCTCCAAGCATCCG
CTGACCATGGCGGCAAGCAAAGCCGTGCTGGAGTACATCAAAACGCATCCGGCGCTGTAT
GACGACATCAACCAAAAAACCGCGCGTCTGGCGATGTCGCTAAATACCTGGTTCTCAGCG
ACCGGCACGCCGATTGAGATCGTCTCAGCCGGCAGCCTGTTCCGTTTCAAATTCAACGGT
AACTACGACATCCTCTTCCACCACCTGATGCTGCGCGGCATTTTCATCTGGGAAGGGCGT
AACTGTTTTGTCTCGGTCGCGCACGCGGATGAGGATATCGATCGGTTTATTGCGGCGGTG
AAAGAGAGCGTTAACGCCATGCGCGTGGACGGCTTCTTTGGGGAGGCGGGATTGCGCCCC
GACACGCGTTATGCCGTTGCAGACAGCCAGCAGCGTTTTCTGCAGCTGGCCGCGCAGGAT
GAAAGCGGGCTGTTGGCCGGGACGATCGGCGGCGTGATCGAAACGCCGCGCAATGTAGAT
GGCGACATCATGCGCGCCGCCTGGCAACTGCTGTGCGAGCGCCATGACGCGCTGCGTATG
CAATTCACCGACGCGGGTGACCTTCAGGTGGCCCTGGCGCCCATCGTCGATATCTGCGAG
GAAAACGCTGCGCCTGCCGCCTGTCTCGCTGAATTTGCTGCGCGTCCTTTTGATCTTACC
GCCACGCCGCTGGCTCGCCTGCTGCTGGTCCGCCACGAGGGGAAAACCACGCTCGCCATC
TCGGCACATCATACGGTGGCGGACGGCTGGTCGTTCATGGTGATGCTGCGCGAGCTGTTG
CATCTTTACGATGCCCTGGCGTCGGGTAAACGTCCTGACCTGGCGGCAGCAGCAAGCTAT
CTGCAGGCTATCCGCGCGCAGAACATCGTCAGCGAGGCTGAGCTACCTGCGCGTCTTGCG
GCGCTTCCAGCGCGCCGGGTAACGCCGGAAGTTCTGAGCGTGCAAGATCCCTCCCTGACG
TATCAGGGGCAGCGGCTGGTACAGCGCCTGTCGTATCCGGGCCTGACCGGGCAGCTTCGC
AAAGCCTCCGCCGAACTGCGGGTGACCCGCTTCGCGATGCTGAATGCGCTCTTTACCCTG
ACGCTTGAGAAGGCGTTCGGCCATTCCCCGGTGCCGGTCGGCGTGCCGGATGCCGGGCGG
GACTTCGGGCAGGGGGACGCGCTTGTCGGGCAGTGCGTCAGGCTGTTGCCGCTATGTATC
GACAGCGCTGCCTGCGCGTCGGTGTCCGGGGTCGCCAGGGCAATCCATGACGGTATCCTC
GCGCAGCGCGGCGAGCCCGCACTGCCGTCACGCTGCTTCCACGGTCAGGATGCGCCGCTT
CCGCTGCTGGCGACCTTTAACGTCGAGCCGCATGCTCCGCTGGCTGAGATGCGTCAGTGG
GAAGCCAGCCTGTCGCTGCTCCCGATCGGCGCCGTCGAATTCCCGCTGATGGTTAACATT
CTCGAGACGAAAGAGGGGCTGTCCGTCGAGCTGGATTACCAGATGCGCTATTTCACCGAG
GAACGAGCCCGCGCGCTACTGGAGCACTTCCTGAAGGCGATAACCGTGCTGGCCGAGCAG
GGAGAAGAGGCTGTTGAAGCATTATTCTCGTCGTCAGAGGCGCTGGCCGCATCATGA
Gene ID: m15A12
SEQ ID NO: 4
ATGTCATGGCAATACTTCAAACAGACTTACCTGGTTAAGTTCTGGTCACCTGTCCCGGCC
GTTATCGCGGCAGGCATTCTCTCTACCTACTATTTCGGCATCACCGGCACCTTCTGGGCC
GTGACCGGCGAGTTCACCCGCTGGGGTGGTCAATTGCTACAGCTCGCAGGCGTACATGCC
GAGGAGTGGGGTTACTTTAAACTCATCCATCTGGACGGCACACCGCTCACCCGCATCGAC
GGGATGATGATCGTCGGCATGTTCGGCGGCTGTTTCGCGGCGGCGCTGTGGGCAAACAAC
GTTAAGTTGCGGATGCCCAAAAGCCGCATTCGTATAATGCAGGCGGTGGGCGGCGGTATC
ATTGCCGGGTTTGGCGCCCGTCTGGCGATGGGCTGTAACCTGGCCGCTTTCTTTACGGGG
ATTCCTCAGTTTTCGCTTCACGCCTGGTTCTTTGCTGTCGCCACGGCCATCGGCTCTTAC
TTCGGTGCAAAATTCACCCTGCTACCGCTGTTCCGCATTCCGGTGAAAATGACAAAAGTC
AGCGCAGCGTCTCCGTTAACCCAGAAGCCGGACCAGGCGCGTCGTCGTTTCCGCCTCGGT
ATGCTGGTCTTTTTGGCTATGGTCGCCTGGGCGCTTTGCACTGCGATGAATCAGCCCAAA
CTCGGCCTGGCGATGCTGTTCGGCGTGGGTTTTGGTCTGCTCATTGAACGCGCGCAGATC
TGCTTTACCTCCGCGTTTCGCGATATGTGGATCACCGGCCGTACGATGATGGCAAAGGCG
ATTATCGCCGGGATGGCGGTCAGCGCCATCGGCATCTTCAGCTATGTTCAACTGGGCGTC
GAACCGAAAATCATGTGGGCTGGCCCTAACGCCGTGATCGGCGGCCTGCTGTTTGGCTTC
GGGATCGTGCTGGCTGGCGGGTGTGAAACCGGCTGGATGTACCGCGCCGTCGAAGGCCAG
GTACACTACTGGTGGGTGGGTCTGGGAAATGTTATCGGCTCGACGATCCTGGCGTACTTC
TGGGACGATCTCTCCCCGGCGCTGGCCACGAGCTGGGATAAGGTCAACCTGCTGAGCACC
TTCGGCCCCCTCGGCGGCCTGCTGGTCACCTACGCCCTGCTGCTGGTGGCTTTTTTACTG
GTTGTCGCACAGGAGAAACGCTTCTTCCGCCGCGCAAGTGTTAAAACAGAAACCCAGGAG
AATGCTGCATGA
Gene ID: m1C5
SEQ ID NO: 5
ATGAAAAGAACCTATCTCTACAGCATGCTGGCGCTCTGCGTGAGTGCCGCGTGCCATGCA
GAAACGTATCCGGCACCCATTGGCCCGTCTCAGTCAGACTTCGGCGGCGTCGGTTTGCTG
CAAACGCCCACCGCGCGGATGGCGCGCGAAGGGGAAATTAGCCTTAACTACCGTGATAAC
GATCAGTATCGTTACTACTCGGCGTCGGTGCAGCTGTTCCCGTGGCTTGAAACCACGCTG
CGCTACACCGACGTGCGTACGAAACAGTACAGCAGCGTTGATGCGTTCTCCGGCGACCAG
ACCTACAAAGATAAAGCCTTCGACGTCAAGCTGCGCCTGTGGGAAGAGAGCTACTGGATG
CCGCAGGTGTCCGTGGGCGCCAAAGATATCGGTGGTACCGGTCTGTTTGATGCTGAATAC
ATCGTGGCCAGTAAAGCCTGGGGGCCGTTCGACTTCTCGCTCGGCCTGGGATGGGGCTAC
CTGGGCACTGGCGGTAACGTGAAAAATCCGTTTTGCTCCTACAGCGATAAATACTGCTAC
CGCGATAACAGCTATAAGAAAGCGGGTTCCATCAACGGTGACCAGATGTTCCACGGTCCG
GCATCGCTGTTTGGCGGCGTGGAGTATCAAACGCCCTGGCAGCCATTACGCCTGAAGCTG
GAATATGAAGGGAATGACTACTCGCAGGACTTCGCCGGGAAGATTGAGCAGAAGAGCAAG
TTTAACGTCGGCGCCATTTATCGCGTCACCGACTGGGCCGACGTTAACCTCAGCTACGAG
CGCGGCAACACCGTGATGTTTGGCTTCACGCTGCGCACCAACTTTAACGATATGCGGCCA
CACTACAATGATAACGCGCGCCCTGCATACCAGCCGGAGCCGCAGGATGCGATTCTGCAG
CACTCCGTGGTGGCAAACCAGCTGACGCTGCTGAAATACAATGCCGGCCTGGCGGATCCG
AAAATTCAGGTGAAAGGCGATACGCTGTACGTGACCGGCGAGCAGGTGAAATACCGCGAC
TCGCGCGAAGGGATCGAACGCGCTAACCGGATCGTAATGAACGATCTGCCGGAGGGGATC
CGCACGATCCGCGTGACGGAAAACCGCCTTAACCTGCCGCAGGTGACGACGGAAACGGAC
GTTGCCAGCCTCAAGCGCCATCTGGAAGGTGAACCGCTCGGGCATGAAACCGAGCTGGTG
CAAAAACGCGTAGAACCGATCGTGCCGGAGACCACCGAGCAGGGCTGGTATATCGACAAA
TCGCGCTTCGATTTCCATATCGATCCGGTGCTGAACCAGTCCGTCGGCGGGCCGGAAAAC
TTCTACATGTATCAGCTGGGCGTCATGGCGACGGCGGATCTGTGGCTTACCGACCACCTG
CTGACCACCGGTAGCCTGTTCGGCAACATCGCTAATAACTACGACAAGTTCAACTACACC
AACCCGCCAAAAGACTCACAGCTGCCGCGCGTGCGTACTCGCGTGCGTGAATACGTGCAG
AACGATGCTTACGTGAATAACCTGCAGGCCAACTATTTCCAGTACTTCGGCAATGGCTTC
TACGGCCAGGTGTACGGCGGGTATCTGGAAACCATGTACGGCGGCGCGGGGGCGGAAGTG
CTTTATCGTCCTGTCGACAGCAACTGGGCGTTCGGGGTTGATGCCAACTACGTCAAGCAG
CGTGACTGGCGCAGCGCGCAGGACATGATGAAGTTCACCGACTACAGCGTCAAAACGGGC
CATCTGACCGCCTACTGGACGCCGTCGTTCGCGCCTGACGTGCTGGTGAAAGCCAGCGTT
GGTCAGTACCTGGCGGGCGATAAGGGCGGTACGCTGGATATCTCTAAACACTTCGACAGC
GGCGTCGTGGTGGGCGGCTATGCCACCATCACCAACGTTTCGCCGGACGAATACGGGGAA
GGGGACTTCACCAAAGGGGTCTACGTGTCGATTCCGCTGGATCTGTTCTCGTCAGGCCCA
ACCCGCAGCCGTGCGGCAGTAGGCTGGACGCCGCTGACGCGTGACGGGGGTCAACAGCTT
GGACGTAAGTTCCAGCTGTATGACATGACGAGCGATAAGAACATTAACTTCCGCTGA
Gene ID: m3H2
SEQ ID NO: 6
ATGAACAAAAGAACATTACTCAGTGTTCTTATTGCGGGCGCATGTGTTGCACCGTTTATG
GCTCAGGCAACCCTGCTGCAGGCCAGCAGCGAACCTTACACCCTTAAGGCCAGCGATCTG
CAGAAGAAAGAGCAGGAGTTAACCAACTTCCCGCTGATGGCTTCGGTGAAGTCAACCATC
CGCACGCTGGACAACAGCCTGGTGGAACAAATTGAGCCTGGTAAATCGACAAACCCGGAA
AACGTTAAGCGCGTGGAAGGGATTATCAAGGCCAGCGACTGGGATTATCTCTTCCCACTG
CGTGCGCCGGAATATACGTACAGCAACTTCCTGAAAGCGGTCGGTAAATTCCCGGCACTG
TGCCAGACCTATACCGATGGTCGTAACAGCGATGCCATCTGCCGTAAGTCTCTGGCAACC
ATGTTCGCGCACTTCGCGCAGGAAACTGGCGGCCACGAGTCCTGGCGTCCGGAAGCCGAG
TGGCGTCAGGCGCTGGTTTACGTGCGTGAGATGGGCTGGAGCGAAGGTCAGAAGGGCGGA
TATAACGGCGAATGTAACCCGGATGTCTGGCAGGGGCAGACCTGGCCGTGCGGTAAAGAC
AAAGATGGCGATTTCGTTAGCTATTTTGGCCGCGGTGCGAAACAGCTCTCCTATAACTAC
AACTACGGCCCGTTCTCTGAAGCGATGTACGGCGACGTCCGCGTACTGCTGGACAAACCT
GAGCTGGTGGCGGACACCTGGCTGAACCTTGCTTCCGCGATCTTCTTCTTCGCCTACCCG
CAGCCGCCAAAACCAAGCATGCTGCAGGTTATCGACGGCACATGGCAGCCGAACGATCAC
GATAAGGCGAATGGTCTGGTACCGGGCTTCGGAGTGACCACGCAGATCATCAACGGCGGC
GTGGAGTGTGGCGGCCCGACTGAGATCGCCCAGTCTCAAAACCGTATCAAATACTACAAA
GAGTTCGCCAACTACCTGAAAGTGCCTGTTCCGTCTAACGAAGTGCTGGGCTGCGCCAAC
ATGAAGCAGTTCGACGAAGGCGGTGCTGGCGCGCTGAAGATCTACTGGGAACAGGACTGG
GGATGGAGCGCGGATACCCCGTCAGGCCAGACCTACTCTTGCCAGCTGGTGGGTTACCAG
ACGCCATTCAGCGCCTTTAAAGAGGGTGACTACACCAAATGCGTGAAGCATTTCTTCAAT
GTGAACGTGGTGGGTGAAGACGGGACTTCCGACGGCGGTAGCGTCACGCCAGCCCCGACG
CCGACCCCTGTCGACCCAACGGACGAAGGCAACACCACGCCGGTGCCAGACGATAACACC
CCGGCTCCGGACGATAATACGCCAGCGCCGGTAAACCATGCGCCGGTAGCAAAAATCGCC
GGTCCGGTGGGTGCGGTTGAAGCGGGTAAATCAGTTTCTCTGAACGCGTCTGGCTCGACC
GACGAAGACGGTAACCACCTGACTTATACCTGGACGGCTCCGAACGGCCAGACCGTAAGC
GGCGAAGATAAAGCGATTATTACCTTCAATGCGCCGGAAGTGGCAGCGGCGACGCAGTAC
CCGATCAACCTTACCGTCAGTGACGGCGAGCTGAGCAGCACCACCAGCTATACGCTGAAC
GTACAGGCTAAGCAGACCAACGGCGGCCAGACCGGGACTTACCCGACCTGGACCTCCAAA
ACCAAATGGAAAGCAGGCGATATCGTTAACAACCGCGGCCAGCTGTTCCAGTGCAAACCT
TATCCGTACAGCGGCTGGTGCAATAACGCGCCATCCTACTACGAGCCAGGTAAAGGGATT
GCATGGCAGGACGCGTGGACTGCGCTGTAA
Gene ID: m5D7
SEQ ID NO: 7
ATGGACTTAACCCAGCTTGAAATGTTTAACGCCGTCGCGCTGACGGGCAGCATCACCCAG
GCGGCGCAGAAGGTGCATCGCGTGCCGTCCAACCTGACGACCCGCATCCGCCAGCTGGAA
GCCGATCTTGGCGTTGAGCTGTTTATTCGTGAGAACCAGCGTTTGCGCTTATCTCCCGCC
GGGCATAACTTCCTGCGCTACAGCAGGCAGATCCTCGCCCTGGTGGATGAAGCGCGCATG
GTCGTCGCGGGTGATGAGCCGCAGGGGTTATTTGCCCTCGGCGCGCTGGAAAGCACCGCC
GCGGTGCGCATTCCCGAAACGCTGGCGCAGTTTAACCAGCGCTATCCGCGCATTCAGTTT
GCCCTTTCTACCGGGCCTTCCGGGACGATGATTGATGGCGTACTGGAGGGCACCTTAAGC
GCCGCCTTTGTCGACGGGCCGCTGTCGCACCCGGAGCTGGAGGGCATGCCGGTCTACCGG
GAAGAGATGATGCTAGTCACGCCTGCCGGGCACGCCGAGGTTGCACGCGCCACGCAGGTT
AGCGGCAGCGACGTTTACGCATTTCGCGCGAACTGCTCGTACCGTCGACATCTGGAAAGC
TGGTTTCATGCGGACAGAGCCACGCCTGGCCGCATTCATGAGATGGAGTCCTACCACGGC
ATGCTCGCCTGCGTCATTGCGGGCGCGGGCATCGCGCTGATGCCGCGCTCGATGCTGGAG
AGTATGCCGGGACATCATCAGGTTGAAGCCTGGCCGCTGGCGGAAAACTGGCGCTGGCTT
ACAACCTGGCTGGTGTGGCGGCGCGGGGCGATGACCCGCCAGCTGGAAGCTTTTATAGCG
CTGCTAAACGAACGTCTTCAACCAGCGCCTTCTCCATAA
Gene ID: m4B9
SEQ ID NO: 8
ATGGTCTGGATTGATTACGCCATCATTGCGGTGATTGGTTTTTCCTGTCTGGTTAGCCTG
ATCCGTGGCTTTGTTCGTGAAGCGTTATCGCTGGTGACTTGGGGTTGTGCTTTCTTTGTC
GCCAGTCATTACTACACTTACCTGTCTGTCTGGTTCACGGGCTTTGAAGATGAACTGGTC
CGAAATGGAATCGCTATCGCGGTGCTGTTTATCGCAACGCTGATTGTCGGCGCTATCGTG
AATTACGTGATAGGTCAGCTGGTCGAGAAAACCGGTCTGTCAGGAACGGACAGGGTACTC
GGGATCTGTTTCGGGGCGTTGCGAGGCGTGCTGATTGTGGCCGCGATCCTGTTCTTCCTG
GATACCTTTACCGGGTTCTCCAAAAGCGAAGACTGGCAGAAATCGCAGCTCATTCCAGAG
TTCAGCTTCATCATCAGATGGTTCTTTGACTATCTGCAAAGCTCGTCGAGTTTTTTGCCC
AGGGCATAA
Gene ID: m1H3
SEQ ID NO: 9
ATGACGAGCCGTAAACCTGCCCATCTTTTACTGGTGGATGACGATCCCGGGCTGTTAAAG
CTGCTGGGGATGCGTCTGGTGAGTGAAGGCTACAGCGTCGTGACCGCCGAAAGCGGGCTG
GAGGGGCTGAAGATCCTCACCCGCGAGAAAATCGATCTGGTGATAAGCGACCTGCGGATG
GACGAAATGGATGGCCTGCAGCTGTTCGCGGAGATCCAGAGGCAGCAGCCGGGTATGCCC
GTGATCATTCTGACGGCGCACGGGTCGATCCCGGATGCGGTTGCCGCGACGCAGCAGGGG
GTCTTCAGCTTCCTGACCAAGCCGGTGGACAAAGACGCGCTGTATAAGGCTATCGACAGC
GCGCTGGAACATGCCGCCCCGGCGGGGGATGAAGCGTGGCGGGAGTCCATCGTCACCCGC
AGCCCCATTATGCTGCGTCTCCTTGAACAGGCCCGGATGGTGGCGCAGTCCGACGTCAGC
GTGCTCATCAACGGCCAGAGCGGAACCGGGAAAGAGATCCTGGCCCAGGCGATCCATAAC
GCCAGCCCGCGCAGTAAAAATGCCTTTATCGCCATTAACTGCGGCGCGCTTCCGGAACAG
CTTCTCGAATCTGAACTGTTTGGTCATGCCCGCGGCGCATTCACCGGCGCGGTGAGCAGT
CGGGAAGGGCTGTTCCAGGCGGCGGAAGGCGGCACGCTGTTCCTGGACGAGATTGGCGAC
ATGCCCGCGCCGCTGCAGGTCAAACTGCTGCGCGTATTGCAGGAGCGAAAAGTTCGCCCG
CTGGGCAGCAACCGCGATATCGATATTAACGTGCGCATTATTTCCGCCACCCACCGCGAC
TTGCCAAAAGTGATGGCCCGCAACGAGTTTCGCGAAGATCTCTACTACCGTCTGAACGTG
GTGAATCTGAAGATCCCTGCGCTGGCGGAGCGCGCGGAAGACATTCCGCTGCTGGCGAAT
CATCTTCTGCGCCAGGCGGCCGATCGTCATAAACCGTTTGTGCGCGCGTTTTCCACCGAC
GCGATGAAGCGGCTGATGGCCGCAGGCTGGCCGGGTAACGTGCGCCAGCTGGTGAACGTG
ATTGAGCAGTGCGTGGCGCTGACCTCCTCACCGGTAATCAGCGATGCGCTTGTAGAGCAG
GCGCTGGAAGGAGAAAACACGGCGCTGCCGACGTTTGCGGAAGCGCGGAATCAGTTCGAG
CTGAACTATCTGCGCAAGCTATTGCAGATCACCAAAGGCAACGTGACCCACGCGGCGCGC
ATGGCCGGACGCAACCGCACCGAGTTCTACAAGCTGCTGTCGCGCCACGAGCTGGAAGCA
AACGATTTTAAAGAGTAA
Gene ID: m10A8
SEQ ID NO: 10
ATGACCCACAACATTTCTCTCAGAAATAGGGTGTGCGATGAACAAAAGTTTGTCATCTCA
GCCTGCGGTTTTACGTTCCAGGGATATCGTCTGTTCCTTAACCAGTACGGAATACAGGCT
TCGCATATTCATTTTGATGGGGATGAGGCATCGCAACAGGATATGAAAAATATCCTTATA
AATCAGAATGCTCATGTTGTGGTGTTTCTCGGAAAAGGTATCTTAAGTCTCCTGGAGAGC
CTGAAGCGACTGGCGTCCGTGCTCAATGCGTTGCCCGTTATTCGACGCGTCACGCTGTAT
GGCGACATACCGGATGGCTGGCTATATCGCACCCTGGGCAGTCTTTTAAATAATAGTTAT
CAATTATCATTGATTCGACTAGCCCGCGTTTCGGATGTCGTCACTGGTTCTCATACGCAC
CATCATGTATTTAAGGAACGTTCGTACTTATTACGCGATCGCTACAGGGATAATTCTTCG
CAAGACAACGTCAAGTGGCTAACAAAAAGAGAGATTGACGTTTTATTAAATTTCTACCGC
GGCATGTCCGTAAAAGAAATGTGCGATGAAATGGGACTATCTAATAAAACGGTTTATACC
CACCGTAAGGAAGGCGTGCTGAAATTACGCTTAATTAAGCGGTGGCTACACGATTCGCAC
AATATCAATGCGGAAAGAAGTATCAAGCGGCGGAGTCAAAACACGGAATTTACGGATAAG
GAAGCAGAGATTTTTAATGCATTATATAAAAAAGAGATCTTCCCTGCTTATCAGATCATT
ACCGATCGTGACAAAAAAGGCGTAGGCTTTGAGATACTGATCCGCTGGAACAAAAACGGT
AAAATTGTCAAGCCGACCAGTTTTCTCACGGATATTTCAAATCATGAGATATGGTTGAAA
ATTACCGCGCTAGTTATTCATGCCGCCGTGTCGGGAATTAATAAGTATAATGGTAAATAC
TATTTTTCTGTGAATATTCCACCTCGCCTGGCCTCCGGAAATGCATTGCCTGATATGGCA
AGGAAAGCTATCGACATGCTGCTCAAACCGCAGTGGGCCGAGAAGCTGGTCTTTGAATTC
GCAGAAGATATTGACGTGACGAAGGACAAAGGGATCCCAGAAACCATGCGGCATTTGCGT
AATACAGGGTGTCGATTGTTTCTGGATGACTGCTTCTCTAATCACCAAACCATGTTCCCG
GTGCGGCAGGTGCATTTTGATGGACTCAAGCTGGATCGGGATATCGTTGAGCATTTTGTG
GCAAACGACAATGACTATAACCTGATCAAAGCGATACAGATTTATAGCGACATGACCGGA
ACGGACTGTATTGCAGAGGGAGTAGACAGTGAGGAAAAATTTGAAAAATTAGTCGCGCTG
GGCGTCAAAAACTTTCAGGGATATTATTTATCGCGAGCCGTGAAAGAGGACGAGTTGGAT
CGCATGGTCAGAATTTTTAGTTAA
Gene ID: m2D7
SEQ ID NO: 11
ATGAAGCCTGAAGAGTTCCGCGCTGATGCCAAACGCCCGTTAACCGGCGAAGAGTATTTA
AAAAGCCTGCAGGACGGTCGTGAGATTTATATCTACGGCGAGCGCGTCAAAGACGTCACC
ACCCATCCGGCATTTCGCAACGCGGCGGCCTCCATCGCGCAGATGTACGACGCGCTGCAC
AAGCCTGACATGCAGGACGCGCTCTGCTGGGGCACCGACACCGGCAGCGGCGGCTATACC
CACAAGTTTTTCCGCGTGGCGAAAAGTGCCGACGACCTGCGCCAGCAGCGCGACGCCATC
GCCGAGTGGTCGCGCCTGAGCTACGGCTGGATGGGCCGCACGCCGGACTACAAAGCCGCG
TTCGGCTGCGCGCTCGGCGCCAACCCGGCCTTCTACGGCCAGTTCGAACAGAACGCCCGT
AACTGGTACACGCGCATTCAGGAAACCGGCCTGTACTTTAACCACGCCATCGTTAACCCG
CCGATCGACCGTCACAAGCCGGCGGACGAGGTGAAAGACGTCTACATCAAGCTGGAGAAA
GAGACCGACGCCGGGATTATCGTCAGCGGCGCGAAAGTGGTAGCCACCAACTCCGCCCTG
ACCCACTACAACATGATCGGCTTCGGCTCCGCGCAGGTGATGGGCGAAAACCCGGACTTC
GCGCTGATGTTCGTCGCGCCGATGGATGCCGAAGGCGTGAAGCTGATCTCCCGCGCCTCT
TACGAAATGGTGGCAGGCGCAACCGGATCCCCGTACGACTACCCGCTCTCCAGCCGCTTT
GATGAGAACGACGCGATCCTGGTGATGGATCACGTGCTGATCCCGTGGGAAAATGTGCTG
ATCTACCGCGATTTCGACCGCTGCCGTCGCTGGACGATGGAAGGCGGTTTCGCGCGGATG
TACCCGCTGCAGGCCTGCGTGCGTCTGGCGGTGAAGCTCGACTTCATCACCGCCCTGCTG
AAGAAATCCCTGGAGTGCACCGGCACCCTGGAGTTCCGCGGCGTACAGGCGGATCTCGGC
GAAGTGGTGGCCTGGCGTAACATGTTCTGGGCGCTGAGCGACTCCATGTGTTCCGAAGCC
ACCCCGTGGGTCAACGGCGCGTATCTGCCGGATCACGCCGCGCTGCAAACCTACCGCGTG
ATGGCGCCGATGGCCTACGCCAAGATCAAGAACATCATCGAGCGTAACGTGACGTCCGGT
CTGATCTACCTGCCGTCCAGCGCCCGGGATCTGAACAACCCGCAGATCGACCAGTATCTG
GCGAAATACGTGCGCGGCTCCAACGGGATGGATCACGTCGAACGCATCAAGATTTTGAAG
CTGATGTGGGATGCCATCGGCAGCGAGTTTGGCGGTCGTCACGAGCTATACGAAATCAAC
TACTCCGGCAGCCAGGATGAGATCCGCCTGCAGTGTCTGCGCCAGGCGCAGAGCTCCGGC
AACATGGACAAAATGATGGCGATGGTCGACCGCTGCATGTCCGAATACGACCAGCACGGC
TGGACCGTACCGCACCTGCACAACAACAGCGATATCAACATGCTCGACAAGCTGCTGAAA
TAG
Gene ID: m1B3
SEQ ID NO: 12
V N T I G I N S E P I L T H S G F S I T A D T T L A A D R H
Y D V I Y L P A L W R N P R A V V R Q Q P E L L A W L S E Q
A A R G T R I A A V G T G C C F L A E S G L L N G K P A T T
H W H Y F K Q F S R D Y P N V K L Q T K H F L T Q A D N I Y
C A A S V K A L S D L T I H F I E T I Y G K R V A T H T Q R
T F F H E I R S Q F D R Q C Y S E E N K P H P D E D I V Q I
Q I W I K A N C A S D I S M Q N L A D M A G M S L R N F N R
R F K N A T D I S P L Q Y L L T A R I E S A M T M L Q S T N
L S I Q E I A N A V G Y Q D I A H F N R Q F K H K T T V S P
G D Y R K I V R A K M F S A Stop
Gene ID: m9F12
SEQ ID NO: 13
M P A K S S G S A W E R F A G V L R N A Q T E C I V T T A K
G A E T L G Q L S L P L S P L I F T F D K P D T A A L P A G
Y R L H P L D R T F S G A F H P V P V A E N D L A F L Q Y T
S G S T G S P K G V M V T H G N L W A N S H A I H R F F G H
H S E S R G T I W L P H F H D M G L I G G L L Q P V F G A F
P C R V M S P M M L M K N P L N W L K H I S D Y Q A T T S G
G P N F A Y D L C V R K I G R E Q V E A L D L S R W D V A F
C G A E P I R P A T L Q Q F S E H F A P A G F R P G A F L P
C Y G M A E T T L I V T G M E K G Q G L R V S D E A G T V S
C G Q A L P D T E V R I V D P D R H Q P L A D G E S G E I W
L R G P S V A A G Y W D N D A A T R E T F Q A S L A G H P H
P W L R S G D M G F L Q S G H L Y V T G R L K E L L I I N G
Q N H Y P T D I E E T I R Q A D P A L A E A T V C V F A S E
D E R P V A L L E L M T R H K N D L D M A T L A P S V T A A
V A E R H G I T L D E L L L V G R R A I P R T T S G K L Q R
T R A K A M H Q Q G T L E V A W R S C Q D A S K P V E L A G
E T P P A L A A L I A G I I S S A M N T T I G E S Q W D E A
F T G F G M S S L Q A V G V I G E L E Q R L G R E L S P A L
I Y D Y P T I N R L A A A L G Q P A A A R P V S S A V A E S
A I A V I G I G V E L P G H S G V E A L W S L L Q Q G H S T
T G E I P A H R W R T S S L D G F N R K G S F F D E V D A F
D A G Y F G I S P R E A V Y I D P Q H R L L L E T V Q Q A L
T D A G L K A S S L R G S D T A V F V G I S A S D Y A L A C
G D N V S A Y S G L G N A H S I A A N R I S Y L Y D L K G P
S V A V D T A C S S S L V A I E G A M Q S L R A G R C A L A
I A G G V N L A L T P H L Q K V F T E A Q M L A P D G R C K
T F D A R A D G Y V R G E G C G V V V L K P L S Q A L A D G
D R V Y A T L V A S A V N Q D G R S N G I T A P N G P S Q Q
A V I L Q A M A D A G L D S D S I D Y I E A H G T G T A L G
D L I E Y Q A L E A V F A D R K K T A P V Q V G S I K T N I
G H L E A A A G V L G V V K T S L M L H F R Q Y V P H L N F
Q Q K N P H I A A I S R H V E V S G A Q P A S W H A D G E A
R Y A G V S S F G F G G T N G H V I L R S A P A V E K R Q E
P A A P H G L L L V G S H D K G A F T L Q R E A V K K G L S
T C Q E S D I A T W C R L V N T R Y D A A R Y R G V A Y G A
D R S Q L A E S L A Q L T V C K V G K A Q P Q V W L F P G Q
G T Q Q I G M G A E L Y H H L P H Y R T Q F D A L A T T I Q
Q R Y Q I D I T Q A L F A R D D S W Q R C A R T C Q L S L F
A C S Y A L A Q S V M Q F G P R P A A V M G H S L G E Y C A
A V I A G Y L S L D D G L A M V H Q R A L L M S A L T Q E G
A M A V V F S G E A D V R Q M I S P W T G D I D I A A F N T
P T L T T I A G S R A A I D A C L Q A I S S K G G H A R K I
K T A S A F H S S M M D P I L G A W R E W L V N N V T F T R
G T I P F Y S N L N G E A C D R T D A D Y W T R Q I R Q P V
S F L Q G V Q N V L A Q G E F T F I D L S A D G S L G K F V
T A T D R R H R V L A A G D R R H E Y K S L L T L L G T L W
Q Q G H D I N W S G L Y H A T T R E A L T L P A I Q F C R K
R Y W L A G E T P A Q T P S A K E D A M S N Q H H L A A E I
K A I I A G F L E A D P A A L D D S L P F L E M G A D S L V
L L D A I N T I K D R F G V A I P V R A L F E E L N T L D A
V I G Y V V E H A Q P A A S L T T P E T A G L A A Q P V A A
P Q G T S R P V P D T V Q D L I A R Q L E L M S Q Q L N L L
N G T A Q A L P M P A A P A T P D V I A P A P V V A P T A P
V K A S A H N S W F K K E T K K V S L G A E R D Q H L A Q L
T E R F V D K T G G S K R N A Q Q Y R A V L A D N R A S A G
F R L S T K E M L Y P L V G E R S Q G S R I W D V D G N E Y
I D F T M G F G A N L L G H A P D C V Q Q A V A D Q L A R
G M Q I G P Q S A L A G E V A T L I S E L T G Q Q R V A F C
N S G S E A V M S A V R L A R A V T G K N K V A L F S G S Y
H G V F D G I L G R Q Q G G E T P E R A T P I A A G T P P S
L V D D L L V L D Y G S E E S L A L I A R Y A A E L A V V I
V E P V Q S R Y P D H Q P R D Y L H T L R E L T T T H N I A
L M F D E V I T G F R L A A G G A Q A Y Y G V Q A D I A S Y
G K I V G G G M P I G V I A G S A R F M D S I D G G F W Q Y
G D D S W P Q A E L I F F A G T F S K H P L T M A A S K A V
L E Y I K T H P A L Y D D I N Q K T A R L A M S L N T W F S
A T G T P I E I V S A G S L F R F K F N G N Y D I L F H H
L M L R G I F I W E G R N C F V S V A H A D E D I D R F I A
A V K E S V N A M R V D G F F G E A G L R P D T R Y A V A D
S Q Q R F L Q L A A Q D E S G L L A G T I G G V I E T P R N
V D G D I M R A A W Q L L C E R H D A L R M Q F T D A G D L
Q V A L A P I V D I C E E N A A P A A C L A E F A A R P F D
L T A T P L A R L L L V R H E G K T T L A I S A H H T V A D
G W S F M V M L R E L L H L Y D A L A S G K R P D L A A A A
S Y L Q A I R A Q N I V S E A E L P A R L A A L P A R R V T
P E V L S V Q D P S L T Y Q G Q R L V Q R L S Y P G L T G Q
L R K A S A E L R V T R F A M L N A L F T L T L E K A F G H
S P V P V G V P D A G R D F G Q G D A L V G Q C V R L L P L
C I D S A A C A S V S G V A R A I H D G I L A Q R G E P A L
P S R C F H G Q D A P L P L L A T F N V E P H A P L A E M R
Q W E A S L S L L P I G A V E F P L M V N I L E T K E G L S
V E L D Y Q M R Y F T E E R A R A L L E H F L K A I T V L A
E Q G E E A V E A L F S S S E A L A A S Stop
Gene ID: m15A12
SEQ ID NO: 14
M S W Q Y F K Q T Y L V K F W S P V P A V I A A G I L S T Y
Y F G I T G T F W A V T G E F T R W G G Q L L Q L A G V H A
E E W G Y F K L I H L D G T P L T R I D G M M I V G M F G G
C F A A A L W A N N V K L R M P K S R I R I M Q A V G G G I
I A G F G A R L A M G C N L A A F F T G I P Q F S L H A W F
F A V A T A I G S Y F G A K F T L L P L F R I P V K M T K V
S A A S P L T Q K P D Q A R R R F R L G M L V F L A M V A W
A L C T A M N Q P K L G L A M L F G V G F G L L I E R A Q I
C F T S A F R D M W I T G R T M M A K A I I A G M A V S A I
G I F S Y V Q L G V E P K I M W A G P N A V I G G L L F G F
G I V L A G G C E T G W M Y R A V E G Q V H Y W W V G L G N
V I G S T I L A Y F W D D L S P A L A T S W D K V N L L S T
F G P L G G L L V T Y A L L L V A F L L V V A Q E K R F F R
R A S V K T E T Q E N A A Stop
Gene ID: m1C5
SEQ ID NO: 15
M K R T Y L Y S M L A L C V S A A C H A E T Y P A P I G P S
Q S D F G G V G L L Q T P T A R M A R E G E I S L N Y R D N
D Q Y R Y Y S A S V Q L F P W L E T T L R Y T D V R T K Q Y
S S V D A F S G D Q T Y K D K A F D V K L R L W E E S Y
W M P Q V S V G A K D I G G T G L F D A E Y I V A S K A W G
P F D F S L G L G W G Y L G T G G N V K N P F C S Y S D K Y
C Y R D N S Y K K A G S I N G D Q M F H G P A S L F G G V E
Y Q T P W Q P L R L K L E Y E G N D Y S Q D F A G K I E Q K
S K F N V G A I Y R V T D W A D V N L S Y E R G N T V M F G
F T L R T N F N D M R P H Y N D N A R P A Y Q P E P Q D A I
L Q H S V V A N Q L T L L K Y N A G L A D P K I Q V K G D T
L Y V T G E Q V K Y R D S R E G I E R A N R I V M N D L P E
G I R T I R V T E N R L N L P Q V T T E T D V A S L K R H L
E G E P L G H E T E L V Q K R V E P I V P E T T E Q G W Y I
D K S R F D F H I D P V L N Q S V G G P E N F Y M Y Q L G
V M A T A D L W L T D H L L T T G S L F G N I A N N Y D K F
N Y T N P P K D S Q L P R V R T R V R E Y V Q N D A Y V N N
L Q A N Y F Q Y F G N G F Y G Q V Y G G Y L E T M Y G G A G
A E V L Y R P V D S N W A F G V D A N Y V K Q R D W R S A Q
D M M K F T D Y S V K T G H L T A Y W T P S F A P D V L V K
A S V G Q Y L A G D K G G T L D I S K H F D S G V V V G G Y
A T I T N V S P D E Y G E G D F T K G V Y V S I P L D L F S
S G P T R S R A A V G W T P L T R D G G Q Q L G R K F Q L Y
D M T S D K N I N F R Stop
Gene ID: m3H2
SEQ ID NO: 16
M N K R T L L S V L I A G A C V A P F M A Q A T L L Q A S S
E P Y T L K A S D L Q K K E Q E L T N F P L M A S V K S T I
R T L D N S L V E Q I E P G K S T N P E N V K R V E G I I K
A S D W D Y L F P L R A P E Y T Y S N F L K A V G K F P A L
C Q T Y T D G R N S D A I C R K S L A T M F A H F A Q E T G
G H E S W R P E A E W R Q A L V Y V R E M G W S E G Q K G G
Y N G E C N P D V W Q G Q T W P C G K D K D G D F V S Y F G
R G A K Q L S Y N Y N Y G P F S E A M Y G D V R V L L D K P
E L V A D T W L N L A S A I F F F A Y P Q P P K P S M L Q V
I D G T W Q P N D H D K A N G L V P G F G V T T Q I I N G G
V E C G G P T E I A Q S Q N R I K Y Y K E F A N Y L K V P V
P S N E V L G C A N M K Q F D E G G A G A L K I Y W E Q D W
G W S A D T P S G Q T Y S C Q L V G Y Q T P F S A F K E G D
Y T K C V K H F F N V N V V G E D G T S D G G S V T P A P T
P T P V D P T D E G N T T P V P D D N T P A P D D N T P A P
V N H A P V A K I A G P V G A V E A G K S V S L N A S G S T
D E D G N H L T Y T W T A P N G Q T V S G E D K A I I I F N
A P E V A A A T Q Y P I N L T V S D G E L S S T T S Y T L N
V Q A K Q T N G G Q T G T Y P T W T S K T K W K A G D I V N
N R G Q L F Q C K P Y P Y S G W C N N A P S Y Y E P G K G I
A W Q D A W T A L Stop
Gene ID: m5D7
SEQ ID NO: 17
M D L T Q L E M F N A V A L T G S I T Q A A Q K V H R V P S
N L T T R I R Q L E A D L G V E L F I R E N Q R L R L S P A
G H N F L R Y S R Q I L A L V D E A R M V V A G D E P Q G L
F A L G A L E S T A A V R I P E T L A Q F N Q R Y P R I Q F
A L S T G P S G T M I D G V L E G T L S A A F V D G P L S H
P E L E G M P V Y R E E M M L V T P A G H A E V A R A T Q V
S G S D V Y A F R A N C S Y R R H L E S W F H A D R A T P G
R I H E M E S Y H G M L A C V I A G A G I A L M P R S M L E
S M P G H H Q V E A W P L A E N W R W L T T W L V W R R G
A M T R Q L E A F I A L L N E R L Q P A P S P Stop
Gene ID: m4B9
SEQ ID NO: 18
M V W I D Y A I I A V I G F S C L V S L I R G F V R E A L S
L V T W G C A F F V A S H Y Y T Y L S V W F T G F E D E L V
R N G I A I A V L F I A T L I V G A I V N Y V I G Q L V E K
T G L S G T D R V L G I C F G A L R G V L I V A A I L F F L
D T F T G F S K S E D W Q K S Q L I P E F S F I I R W F F D
Y L Q S S S S F L P R A Stop
Gene ID: m1H3
SEQ ID NO: 19
M T S R K P A H L L L V D D D P G L L K L L G M R L V S E G
Y S V V T A E S G L E G L K I L T R E K I D L V I S D L
R M D E M D G L Q L F A E I Q R Q Q P G M P V I I L T A H G
S I P D A V A A T Q Q G V F S F L T K P V D K D A L Y K A I
D S A L E H A A P A G D E A W R E S I V T R S P I M L R L L
E Q A R M V A Q S D V S V L I N G Q S G T G K E I L A Q A I
H N A S P R S K N A F I A I N C G A L P E Q L L E S E L F G
H A R G A F T G A V S S R E G L F Q A A E G G T L F L D E I
G D M P A P L Q V K L L R V L Q E R K V R P L G S N R D I D
I N V R I I S A T H R D L P K V M A R N E F R E D L Y Y R L
N V V N L K I P A L A E R A E D I P L L A N H L L R Q A A D
R H K P F V R A F S T D A M K R L M A A G W P G N V R Q L V
N V I E Q C V A L T S S P V I S D A L V E Q A L E G E N T A
L P I F A E A R N Q F E L N Y L R K L L Q I T K G N V T H A
A R M A G R N R T E F Y K L L S R H E L E A N D F K E Stop
Gene ID: m10A8
SEQ ID NO: 20
M T H N I S L R N R V C D E Q K F V I S A C G F T F Q G Y R
L F L N Q Y G I Q A S H I H F D G D E A S Q Q D M K N I L I
N Q N A H V V V F L G K G I L S L L E S L K R L A S V L N A
L P V I R R V T L Y G D I P D G W L Y R T L G S L L N N S Y
Q L S L I R L A R V S D V V T G S H T H H H V F K E R S Y L
L R D R Y R D N S S Q D N V K W L T K R E I D V L L N F Y R
G M S V K E M C D E M G L S N K T V Y T H R K E G V L K L R
L I K R W L H D S H N I N A E R S I K R R S Q N T E F T D K
E A E I F N A L Y K K E I F P A Y Q I I T D R D K K G V G F
E I L I R W N K N G K I V K P T S F L T D I S N H E I W L K
I T A L V I H A A V S G I N K Y N G K Y Y F S V N I P P R L
A S G N A L P D M A R K A I D M L L K P Q W A E K L V F E F
A E D I D V T K D K G I P E T M R H L R N T G C R L F L D D
C F S N H Q T M F P V R Q V H F D G L K L D R D I V E H F V
A N D N D Y N L I K A I Q I Y S D M T G T D C I A E G V D S
E E K F E K L V A L G V K N F Q G Y Y L S R A V K E D E L D
R M V R I F S
Gene ID: m2D7
SEQ ID NO: 21
M K P E E F R A D A K R P L T G E E Y L K S L Q D G R E I Y
I Y G E R V K D V T T H P A F R N A A A S I A Q M Y D A L H
K P D M Q D A L C W G T D T G S G G Y T H K F F R V A K S A
D D L R Q Q R D A I A E W S R L S Y G W M G R T P D Y K A A
F G C A L G A N P A F Y G Q F E Q N A R N W Y T R I Q E T G
L Y F N H A I V N P P I D R H K P A D E V K D V Y I K L E K
E T D A G I I V S G A K V V A T N S A L T H Y N M I G F G S
A Q V M G E N P D F A L M F V A P M D A E G V K L I S R A S
Y E M V A G A T G S P Y D Y P L S S R F D E N D A I L V M D
H V L I P W E N V L I Y R D F D R C R R W T M E G G F A
R M Y P L Q A C V R L A V K L D F I T A L L K K S L E C T G
T L E F R G V Q A D L G E V V A W R N M F W A L S D S M C S
E A T P W V N G A Y L P D H A A L Q T Y R V M A P M A Y A K
I K N I I E R N V T S G L I Y L P S S A R D L N N P Q I D Q
Y L A K Y V R G S N G M D H V E R I K I L K L M W D A I G S
E F G G R H E L Y E I N Y S G S Q D E I R L Q C L R Q A Q S
S G N M D K M M A M V D R C M S E Y D Q H G W T V P H L H N
N S D I N M L D K L L K Stop

### REFERENCES

1. National Research Council (1996) Lost Crops of Africa: Volume I: Grains: Finger Millet. The National Academies Press. pp. 39-58.
2. Hilu K W, Wet J M Jd (1976) Domestication of Eleusine coracana. Economic Botany 30: 199-208.
3. Munimbazi C, Bullerman L B (1996) Molds and mycotoxins in foods from Burundi. Journal of Food Protection 59: 869-875.
4. Sutton J C (1982) Epidemiology of wheat head blight and maize ear rot caused by Fusarium graminearum. Canadian Journal of Plant Pathology 4: 195-209.
5. Saleh A A, Esele J, Logrieco A, Ritieni A, Leslie J F (2012) Fusarium verticillioides from finger millet in Uganda. Food Additives & Contaminants: Part A 29: 1762-1769.
6. Pall B, Lakhani J (1991) Seed mycoflora of ragi, Eleusine coracana (L.) Gaertn. Research and Development Reporter 8: 78-79.
7. Amata R, Burgess L, Summerell B, Bullock S, Liew E, et al. (2010) An emended description of Fusarium brevicatenulatum and F. pseudoanthophilum based on isolates recovered from millet in Kenya. Fungal Diversity 43: 11-25.
8. Penugonda S, Girisham S, Reddy S (2010) Elaboration of mycotoxins by seed-borne fungi of finger millet (Eleusine coracana L.). Int J Biotech Mol Biol Res 1: 62-64.
9. Ramana M V, Nayaka S C, Balakrishna K, Murali H, Batra H (2012) A novel PCR-DNA probe for the detection of fumonisin-producing Fusarium species from major food crops grown in southern India. Mycology 3: 167-174.
10. Adipala E (1992) Seed-borne fungi of finger millet. E Afr Agricult Forest J 57: 173-176.
11. Chandrashekar A, Satyanarayana K (2006) Disease and pest resistance in grains of sorghum and millets. Journal of Cereal Science 44: 287-304.
12. Siwela M, Taylor J, de Milliano W A, Duodu K G (2010) Influence of phenolics in finger millet on grain and malt fungal load, and malt quality. Food Chemistry 121: 443-449.
13. Johnston-Monje D, Raizada M N (2011) Conservation and Diversity of Seed Associated Endophytes in across Boundaries of Evolution, Ethnography and Ecology. PLoS ONE 6: e20396.
14. Mousa W K, Raizada M N (2013) The diversity of anti-microbial secondary metabolites produced by fungal endophytes: An interdisciplinary perspective. Frontiers in Microbiology 4:65
15. Haas D, Defago G (2005) Biological control of soil-borne pathogens by fluorescent pseudomonads. Nat Rev Micro 3: 307-319.
16. Waller F, Achatz B, Baltruschat H, Fodor J, Becker K, et al. (2005) The endophytic fungus Piriformospora indica reprograms barley to salt-stress tolerance, disease resistance, and higher yield. Proceedings of the National Academy of Sciences of the United States of America 102: 13386-13391.
17. Johnston-Monje D, Raizada M. N. (2011) Integration of biotechnologies—Plant and Endophyte Relationships Nutrient Management. In: Murray Moo-Young, editor, Comprehensive Biotechnology, Second Edition, Volume 4, pp. 713-727.
18. O'Donnell K, Rooney A P, Proctor R H, Brown D W, McCormick S P, et al. (2013) Phylogenetic analyses of support a middle cretaceous origin for a Glade comprising all agriculturally and medically important Fusaria. Fungal Genet Biol 52: 20-31.
19. Khalil O A K, de Faria Oliveira O M M, Vellosa J C R, de Quadros A U, Dalposso L M, et al. (2012) Curcumin antifungal and antioxidant activities are increased in the presence of ascorbic acid. Food Chemistry 133: 1001-1005.
20. Wang K, Kang L, Anand A, Lazarovits G, Mysore K S (2007) Monitoring in planta bacterial infection at both cellular and whole-plant levels using the green fluorescent protein variant GFPuv. New Phytologist 174: 212-223.
21. Calvin N, Hanawalt P (1988) High-efficiency transformation of bacterial cells by electroporation. Journal of Bacteriology 170: 2796-2801.

1.-12. (canceled)

13. A synthetic combination comprising a purified bacterial population in association with a plurality of seeds of an agricultural plant, wherein the purified bacterial population comprises an endophyte that is heterologous to the seeds and comprises a 16S rRNA nucleic acid sequence at least 96% sequence identical to the sequence set forth in SEQ ID NO:1, and wherein the endophyte is present in the synthetic combination in an amount effective to provide a benefit to the seeds or seedlings or the plants derived from the seeds or seedlings.

14. The synthetic combination of claim 13, wherein the endophyte is an Enterobacter species.

15. The synthetic combination of claim 13, wherein the endophyte inhibits the growth of at least one fungal pathogen.

16.-20. (canceled)

21. The synthetic combination of claim 13, wherein the bacteria comprises at least one gene that is induced as a result of contact with Fusarium mycelium.

22. The synthetic combination of claim 21, wherein the at least one gene has at least 80% sequence identity with a nucleic acid sequence selected from any one of SEQ ID NOs: 2-11.

23. The synthetic combination of claim 13, wherein the benefit is selected from the group consisting of decreased ear rot, decreased kernel rot, decreased head blight, improved growth, increased mass, increased grain yield, and decreased levels of deoxynivalenol.

24. The synthetic combination of claim 13, wherein the agricultural plant is a cereal.

25. The synthetic combination of claim 24, wherein the cereal is maize, wheat, sorghum or barley.

25.-29. (canceled)

30. A method of preventing or inhibiting fungal growth on a plant, comprising contacting the surface of a plurality of seeds or seedlings with a formulation comprising a purified bacterial population that comprises an endophyte that is heterologous to the seeds or seedlings and comprises a 16S rRNA nucleic acid sequence at least 96% sequence identical to the sequence set forth in SEQ ID NO:1, and wherein the endophyte is present in the synthetic combination in an amount effective to prevent or inhibit fungal growth on the plants derived from the seeds or seedlings.

31. The method of claim 30, wherein the endophyte is an Enterobacter species.

32. The method of claim 30, wherein the endophyte inhibits the growth of at least one fungal pathogen.

33.-37. (canceled)

38. The method of claim 30, wherein the endophyte comprises at least one gene that is induced as a result of contact with Fusarium mycelium.

39. The method of claim 38, wherein the at least one gene has at least 80% sequence identity with a nucleic acid sequence selected from any one of SEQ ID NOs: 2-11.

40. The method of claim 30, wherein the agricultural plant is a cereal.

41. The method of claim 40, wherein the cereal is maize, wheat, sorghum or barley.

42.-56. (canceled)

57. A recombinant construct comprising a nucleic acid sequence that has at least 80% sequence identity with a nucleic acid sequence selected from any one of SEQ ID NOs: 2-11, or the complement thereof.

58. (canceled)

59. A transformed bacterial cell, plant cell, plant or plant part expressing a nucleic acid molecule comprising a nucleic acid sequence that has at least 80% sequence identity with a nucleic acid sequence selected from any one of SEQ ID NOs: 2-11, or the complement thereof, wherein said bacterial cell, plant cell, plant or plant part is resistant to fungal infection.

60. (canceled)

61. The transformed bacterial cell, plant cell, plant or plant part of claim 59, wherein said bacterial cell, plant cell, plant or plant part is resistant to fungal infection by Fusarium, optionally F. graminearum.

62.-75. (canceled)


SEQUENCE LISTING (53 sequences)

SEQ ID NO: 1 - DNA, 596 bp, Enterobacter Species
  misc_feature (152)..(152): n is a, c, g, or t
  misc_feature (161)..(162): n is a, c, g, or t
  misc_feature (278)..(279): n is a, c, g, or t
  misc_feature (591)..(591): n is a, c, g, or t
tagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttancagagatgnnttggtgccttcgggaactctgagacaggtgctgcatggctgtcgtcagctcgtgttgtgaaatgttgggttaagtcccgcaacgagcgcaacccttatcctttgttgccagcggtnnggccgggaactcaaaggagactgccagtgataaactggaggaaggtggggatgacgtcaagtcatcatggcccttacgagtagggctacacacgtgctacaatggcgcatacaaagagaagcgacgacctcataaagtgcgtcgtagtccggattggagtctgcaactcgactccatacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacnaaggt

SEQ ID NO: 2 - DNA, 855 bp, Enterobacter Species
gtgaataccattggcataaacagcgagcccatcctgacgcacagtggctttagcattaccgccgataccactcttgccgcagacaggcactatgacgttatctatcttcctgccctgtggcgcaatcctcgtgcagtggtcagacaacagcctgaactcctggcatggcttagcgaacaggcggcgcgagggacccgcatcgcggccgtcggaacgggctgctgtttcctggcggaatcgggattgctcaacgggaaacccgccaccacccactggcactacttcaaacagttctcgcgtgactaccccaacgtaaaattacaaaccaaacattttctcacgcaggccgataatatttactgtgccgccagcgtcaaagccctctcagatctgaccatccatttcatcgaaacgatatacgggaaacgtgtagccacacatacccaacggacctttttccatgaaattcggagtcagtttgatcgccagtgttacagtgaagaaaacaaaccccatccggatgaagatattgttcaaattcaaatctggataaaagccaactgcgcctcggatatatccatgcaaaatcttgccgatatggctggcatgagtttgcgcaactttaatcgccgctttaaaaatgccaccgatatatcccccctgcagtatttattaaccgccagaattgaatccgccatgacaatgctgcaatccaccaatctgagcattcaggagattgcgaatgcggtgggatatcaggatattgcgcactttaatcgccagtttaagcataaaacaacggtttcaccgggggattaccgtaagaccgtccgggcaaagatgtttagtgcataa

SEQ ID NO: 3 - DNA, 7437 bp, Enterobacter Species
atgcccgcaaaatcatcaggaagcgcgtgggagcgttttgccggagtattacgtaatgcgcaaacggaatgtattgttacgacagcaaagggagcagaaacgctaggccagctatcacttccgttatccccgcttatttttaccttcgacaaaccagacacagctgcgctccctgcgggctatcgtctgcatcctcttgatcgcacgttctccggggcatttcaccccgttccggtagcggaaaacgatctggcttttttacagtacacctcgggttcgacgggctctccgaagggcgtgatggtgacccatggcaacctgtgggccaactcgcatgccattcaccgctttttcggccatcacagcgaaagccggggcacgatctggctgccgcattttcatgatatggggctgattggcgggctactgcagcccgtgtttggcgcattcccctgtcgggtgatgtcgcccatgatgttaatgaaaaatccccttaactggctcaaacatatttctgactatcaggcgacgacctccggcggccctaatttcgcctacgatctgtgcgtgcgcaagattggcagagagcaagttgaggcattagatctttctcgctgggatgtggctttttgcggcgcagagccgattcggcccgccacgctacagcagtttagcgaacactttgcgcccgcagggtttcggcccggcgcgttcctgccctgctacggcatggcggaaaccacgctcatcgtcaccgggatggagaaaggacaagggctccgcgtttccgacgaggccggtacggtgagctgcgggcaggctctgccggataccgaggtgcgtatcgtcgatcccgatcgccatcagccgcttgctgatggtgagagcggggaaatatggctgcgtggtccgagcgttgccgcaggatactgggacaatgacgccgccacacgcgaaaccttccaggcgtctctggctggccatccgcacccgtggctgcgcagcggcgatatgggttttctccagtccggccatttgtacgtcaccggacgactcaaagagctgctgattatcaacggtcagaaccactacccgacggatattgaagagacgatccgccaggccgatcccgcgctggcggaagcgacggtttgcgtgtttgccagtgaagacgagcgcccggttgcgctgcttgagctgatgacgcgccataaaaacgatctcgatatggcgacgctggcaccgtccgtgaccgcggcggtggcggagcggcacggcatcacgctggatgaactgctccttgtcgggcgaagggcaattccccgcaccaccagcgggaaactacagcgcacccgcgcgaaagcgatgcaccagcagggaaccctggaagtagcctggcgcagctgccaggacgcgtcgaaacctgttgaactcgcgggggaaaccccacccgcgctggcggcgctgatagccgggataatcagcagcgcgatgaacacgacgatcggcgaatcccagtgggacgaggcgtttaccggctttggcatgagctctctgcaggcggtgggcgtgattggcgagcttgaacagcggctgggccgcgagctctctcccgcgctgatttatgactaccccaccatcaatcggctggcggccgcgctggggcaacccgctgcggcccggccggtcagctcagccgtcgcggagagcgccattgcggtgattggcatcggcgtggagctgccgggacatagcggcgtggaggcgctgtggtcgctgctgcagcagggccacagcacgaccggcgagatcccggcgcaccgctggcgtacctcgtcccttgacggttttaaccgtaaaggcagtttcttcgacgaggtcgacgcgttcgacgcaggctacttcggcatctctccccgtgaggccgtctatatcgatccgcagcatcgtctgctgttagaaaccgttcaacaggcgctaaccgatgccggccttaaggcgtcctccctgcgcggtagcgatacggcggtctttgttgggatcagcgccagcgactacgcgctggcctgcggcgataacgtctcggcctacagcggcttaggcaacgcgcacagtatcgcggccaaccgaatttcttatctttatgatttaaaaggtccaagcgtcgccgtcgacacggcctgttcttcctcgctggtggcgatagagggggcaatgcagagcctgcgggccggacgttgcgctctggccattgccggaggcgttaatctggcgctgacgccacatttgcaaaaagtcttcaccgaagcccagatgctggcccccgacggccggtgtaaaaccttcgacgcccgcgcggatggctatgttcgtggcgaagggtgtggcgtcgtggtgcttaagccgctttcacaggcgctggcggatggcgatcgggtttatgccacgctggtggcgagcgccgtgaatcaggacggccgcagcaacggcattaccgcgccaaatggcccatcgcagcaggcggtcatcctgcaggcgatggcggacgccgggttggacagcgacagcattgactatatcgaagcgcacggtacgggaaccgcgcttggcgatctgattgaatatcaggcgctggaagcggtgtttgcggaccggaaaaagaccgcacctgtccaggtgggttcgatcaaaaccaacattggccaccttgaggcggcggcgggcgtgctgggcgtggtgaaaacgtctctgatgctgcacttccggcaatacgtacctcacctcaattttcagcagaaaaacccgcatattgcggcgattagccgtcatgttgaggtgagcggcgcgcagcctgcctcatggcatgccgatggcgaagcgcgctatgcgggcgtaagcagctttggcttcggcggtaccaacggtcatgtgattttgcgcagcgcgccagcggtggaaaaacgccaggagcccgctgcgccgcacggcctgcttctggtcggttcacatgataaaggggcgtttacccttcagcgggaggcggtcaaaaaagggttatcgacgtgccaggagagcgatattgccacctggtgtcggctggtgaacacccgctacgacgcggcccgctatcgcggcgtggcgtatggcgcggatcgctcccagctggcggaaagccttgcgcagctcaccgtctgcaaggtgggtaaagcccagccccaggtctggctcttcccggggcagggcacccagcaaatcggcatgggtgccgagctgtatcaccatctgccgcactatcgcacccagtttgacgcgctggcgacgactattcagcagcgctatcagattgatattacgcaggcgctgtttgcccgtgacgacagctggcagcgctgcgccagaacgtgtcagctctcattatttgcctgtagctacgcgcttgctcagagcgtgatgcagttcggcccgcgtccggctgccgtaatggggcacagcctgggagagtactgcgcggcggttatcgctggctatctctcgctggacgacgggctggcaatggttcatcagcgcgcgctgttgatgtcagccctgacgcaggaaggggcgatggctgtcgtcttcagcggcgaagccgacgtccgtcagatgatttccccctggacgggcgacattgatattgccgcattcaacacgccgacattgaccaccatcgcaggcagtcgggcggcgattgacgcctgccttcaggccatatcttcaaaaggcggtcacgccagaaaaattaaaactgccagcgcatttcactcctcgatgatggatccgatcctcggcgcctggcgcgagtggctggtcaacaacgtcaccttcacccgcgggacgatcccgttttacagcaacctgaacggtgaggcgtgcgaccgcaccgacgccgactactggacccggcaaattcgccagcccgtgagtttccttcagggcgtgcaaaacgtgctggcacagggtgagttcacctttatcgatctgagcgcggacggttcgctgggcaaatttgtgaccgcaactgaccgccgtcaccgggtgctggccgcaggcgaccggcgacatgagtacaaatcactgctgacgctgctgggtacgctgtggcagcaagggcacgacatcaactggagcgggctgtaccacgcgaccacgcgggaggcgctaaccctgcccgccatccagttctgccgcaaacgttactggctggcgggtgagacgccagcgcagaccccatctgcaaaagaggacgctatgtcaaatcaacaccatttagccgctgaaataaaagcgattattgccggttttcttgaggcggatcccgccgcgcttgacgactctctgccgttcctggaaatgggggcggactcgctggtgctgctggatgccatcaataccattaaagaccgctttggcgtagccatcccggtgcgggcgctgtttgaagagctcaatacgctggacgcggtgatcggatatgtggtggagcacgcgcagccggcggcttcgctcaccaccccggaaaccgccggcctggcggcacagcctgtcgcggcaccgcagggtaccagcaggccggtgcctgatacggttcaggatctgattgcccgccagctggagctcatgtcccagcagctaaatttgcttaacggcacggcgcaggctctcccgatgccagccgcacccgcgacgccggacgttatcgcgcctgcgcccgtcgtggccccgaccgcaccggtgaaggccagcgcgcacaacagctggtttaaaaaagagaccaaaaaggtctccctcggcgctgagcgcgaccagcatctggcgcaactcaccgaacggtttgtcgataaaacgggcgggtcaaaacgcaatgcccagcaataccgcgccgtgctcgcggataaccgggcctctgcgggctttcgtttatccaccaaagagatgctttaccccttagtgggagagcgctctcagggttcgcgcatctgggatgtcgatggcaacgagtatattgattttacgatggggtttggcgctaacctgctgggacatgcgccggactgcgtgcagcaggccgttgccgaccagctggcgcggggcatgcagattggtccgcaaagcgccctggcgggcgaggtggcaacgcttatcagcgagctgaccggccagcagcgcgtggcattttgtaactccggctccgaagcggtaatgagcgccgtgcgcctggcgcgggccgtgacggggaaaaataaagtcgcgctgtttagcggctcgtatcatggcgtgtttgacggcattctggggcgacagcaggggggagaaacccccgagcgcgcgacgccgattgccgcaggcacgccgccatcgctggtggacgacctgctggtgctcgactacggcagcgaagagagcctggcgcttatcgcccgctatgccgccgagttggcggtggtgatcgtcgaaccggtgcagagccgttatcccgatcatcagccacgcgactatctgcataccctgcgtgaactgacgacaacccacaacattgcgctgatgtttgacgaggtgatcaccggtttccgtctggctgccggcggcgcgcaggcgtattacggcgttcaggcggatatcgcctcctacggaaagattgtcggcggcggtatgccgatcggcgtgattgcaggctctgcgcgctttatggacagtatcgacggcggattctggcagtatggcgatgactcctggccgcaggctgaactgatcttctttgccggtacgttctccaagcatccgctgaccatggcggcaagcaaagccgtgctggagtacatcaaaacgcatccggcgctgtatgacgacatcaaccaaaaaaccgcgcgtctggcgatgtcgctaaatacctggttctcagcgaccggcacgccgattgagatcgtctcagccggcagcctgttccgtttcaaattcaacggtaactacgacatcctcttccaccacctgatgctgcgcggcattttcatctgggaagggcgtaactgttttgtctcggtcgcgcacgcggatgaggatatcgatcggtttattgcggcggtgaaagagagcgttaacgccatgcgcgtggacggcttctttggggaggcgggattgcgccccgacacgcgttatgccgttgcagacagccagcagcgttttctgcagctggccgcgcaggatgaaagcgggctgttggccgggacgatcggcggcgtgatcgaaacgccgcgcaatgtagatggcgacatcatgcgcgccgcctggcaactgctgtgcgagcgccatgacgcgctgcgtatgcaattcaccgacgcgggtgaccttcaggtggccctggcgcccatcgtcgatatctgcgaggaaaacgctgcgcctgccgcctgtctcgctgaatttgctgcgcgtccttttgatcttaccgccacgccgctggctcgcctgctgctggtccgccacgaggggaaaaccacgctcgccatctcggcacatcatacggtggcggacggctggtcgttcatggtgatgctgcgcgagctgttgcatctttacgatgccctggcgtcgggtaaacgtcctgacctggcggcagcagcaagctatctgcaggctatccgcgcgcagaacatcgtcagcgaggctgagctacctgcgcgtcttgcggcgcttccagcgcgccgggtaacgccggaagttctgagcgtgcaagatccctccctgacgtatcaggggcagcggctggtacagcgcctgtcgtatccgggcctgaccgggcagcttcgcaaagcctccgccgaactgcgggtgacccgcttcgcgatgctgaatgcgctctttaccctgacgcttgagaaggcgttcggccattccccggtgccggtcggcgtgccggatgccgggcgggacttcgggcagggggacgcgcttgtcgggcagtgcgtcaggctgttgccgctatgtatcgacagcgctgcctgcgcgtcggtgtccggggtcgccagggcaatccatgacggtatcctcgcgcagcgcggcgagcccgcactgccgtcacgctgcttccacggtcaggatgcgccgcttccgctgctggcgacctttaacgtcgagccgcatgctccgctggctgagatgcgtcagtgggaagccagcctgtcgctgctcccgatcggcgccgtcgaattcccgctgatggttaacattctcgagacgaaagaggggctgtccgtcgagctggattaccagatgcgctatttcaccgaggaacgagcccgcgcgctactggagcacttcctgaaggcgataaccgtgctggccgagcagggagaagaggctgttgaagcattattctcgtcgtcagaggcgctggccgcatcatga

SEQ ID NO: 4 - DNA, 1212 bp, Enterobacter Species
atgtcatggcaatacttcaaacagacttacctggttaagttctggtcacctgtcccggccgttatcgcggcaggcattctctctacctactatttcggcatcaccggcaccttctgggccgtgaccggcgagttcacccgctggggtggtcaattgctacagctcgcaggcgtacatgccgaggagtggggttactttaaactcatccatctggacggcacaccgctcacccgcatcgacgggatgatgatcgtcggcatgttcggcggctgtttcgcggcggcgctgtgggcaaacaacgttaagttgcggatgcccaaaagccgcattcgtataatgcaggcggtgggcggcggtatcattgccgggtttggcgcccgtctggcgatgggctgtaacctggccgctttctttacggggattcctcagttttcgcttcacgcctggttctttgctgtcgccacggccatcggctcttacttcggtgcaaaattcaccctgctaccgctgttccgcattccggtgaaaatgacaaaagtcagcgcagcgtctccgttaacccagaagccggaccaggcgcgtcgtcgtttccgcctcggtatgctggtctttttggctatggtcgcctgggcgctttgcactgcgatgaatcagcccaaactcggcctggcgatgctgttcggcgtgggttttggtctgctcattgaacgcgcgcagatctgctttacctccgcgtttcgcgatatgtggatcaccggccgtacgatgatggcaaaggcgattatcgccgggatggcggtcagcgccatcggcatcttcagctatgttcaactgggcgtcgaaccgaaaatcatgtgggctggccctaacgccgtgatcggcggcctgctgtttggcttcgggatcgtgctggctggcgggtgtgaaaccggctggatgtaccgcgccgtcgaaggccaggtacactactggtgggtgggtctgggaaatgttatcggctcgacgatcctggcgtacttctgggacgatctctccccggcgctggccacgagctgggataaggtcaacctgctgagcaccttcggccccctcggcggcctgctggtcacctacgccctgctgctggtggcttttttactggttgtcgcacaggagaaacgcttcttccgccgcgcaagtgttaaaacagaaacccaggagaatgctgcatga

SEQ ID NO: 5 - DNA, 2097 bp, Enterobacter Species
atgaaaagaacctatctctacagcatgctggcgctctgcgtgagtgccgcgtgccatgcagaaacgtatccggcacccattggcccgtctcagtcagacttcggcggcgtcggtttgctgcaaacgcccaccgcgcggatggcgcgcgaaggggaaattagccttaactaccgtgataacgatcagtatcgttactactcggcgtcggtgcagctgttcccgtggcttgaaaccacgctgcgctacaccgacgtgcgtacgaaacagtacagcagcgttgatgcgttctccggcgaccagacctacaaagataaagccttcgacgtcaagctgcgcctgtgggaagagagctactggatgccgcaggtgtccgtgggcgccaaagatatcggtggtaccggtctgtttgatgctgaatacatcgtggccagtaaagcctgggggccgttcgacttctcgctcggcctgggatggggctacctgggcactggcggtaacgtgaaaaatccgttttgctcctacagcgataaatactgctaccgcgataacagctataagaaagcgggttccatcaacggtgaccagatgttccacggtccggcatcgctgtttggcggcgtggagtatcaaacgccctggcagccattacgcctgaagctggaatatgaagggaatgactactcgcaggacttcgccgggaagattgagcagaagagcaagtttaacgtcggcgccatttatcgcgtcaccgactgggccgacgttaacctcagctacgagcgcggcaacaccgtgatgtttggcttcacgctgcgcaccaactttaacgatatgcggccacactacaatgataacgcgcgccctgcataccagccggagccgcaggatgcgattctgcagcactccgtggtggcaaaccagctgacgctgctgaaatacaatgccggcctggcggatccgaaaattcaggtgaaaggcgatacgctgtacgtgaccggcgagcaggtgaaataccgcgactcgcgcgaagggatcgaacgcgctaaccggatcgtaatgaacgatctgccggaggggatccgcacgatccgcgtgacggaaaaccgccttaacctgccgcaggtgacgacggaaacggacgttgccagcctcaagcgccatctggaaggtgaaccgctcgggcatgaaaccgagctggtgcaaaaacgcgtagaaccgatcgtgccggagaccaccgagcagggctggtatatcgacaaatcgcgcttcgatttccatatcgatccggtgctgaaccagtccgtcggcgggccggaaaacttctacatgtatcagctgggcgtcatggcgacggcggatctgtggcttaccgaccacctgctgaccaccggtagcctgttcggcaacatcgctaataactacgacaagttcaactacaccaacccgccaaaagactcacagctgccgcgcgtgcgtactcgcgtgcgtgaatacgtgcagaacgatgcttacgtgaataacctgcaggccaactatttccagtacttcggcaatggcttctacggccaggtgtacggcgggtatctggaaaccatgtacggcggcgcgggggcggaagtgctttatcgtcctgtcgacagcaactgggcgttcggggttgatgccaactacgtcaagcagcgtgactggcgcagcgcgcaggacatgatgaagttcaccgactacagcgtcaaaacgggccatctgaccgcctactggacgccgtcgttcgcgcctgacgtgctggtgaaagccagcgttggtcagtacctggcgggcgataagggcggtacgctggatatctctaaacacttcgacagcggcgtcgtggtgggcggctatgccaccatcaccaacgtttcgccggacgaatacggggaaggggacttcaccaaaggggtctacgtgtcgattccgctggatctgttctcgtcaggcccaacccgcagccgtgcggcagtaggctggacgccgctgacgcgtgacgggggtcaacagcttggacgtaagttccagctgtatgacatgacgagcgataagaacattaacttccgctga

SEQ ID NO: 6 - DNA, 1830 bp, Enterobacter Species
atgaacaaaagaacattactcagtgttcttattgcgggcgcatgtgttgcaccgtttatggctcaggcaaccctgctgcaggccagcagcgaaccttacacccttaaggccagcgatctgcagaagaaagagcaggagttaaccaacttcccgctgatggcttcggtgaagtcaaccatccgcacgctggacaacagcctggtggaacaaattgagcctggtaaatcgacaaacccggaaaacgttaagcgcgtggaagggattatcaaggccagcgactgggattatctcttcccactgcgtgcgccggaatatacgtacagcaacttcctgaaagcggtcggtaaattcccggcactgtgccagacctataccgatggtcgtaacagcgatgccatctgccgtaagtctctggcaaccatgttcgcgcacttcgcgcaggaaactggcggccacgagtcctggcgtccggaagccgagtggcgtcaggcgctggtttacgtgcgtgagatgggctggagcgaaggtcagaagggcggatataacggcgaatgtaacccggatgtctggcaggggcagacctggccgtgcggtaaagacaaagatggcgatttcgttagctattttggccgcggtgcgaaacagctctcctataactacaactacggcccgttctctgaagcgatgtacggcgacgtccgcgtactgctggacaaacctgagctggtggcggacacctggctgaaccttgcttccgcgatcttcttcttcgcctacccgcagccgccaaaaccaagcatgctgcaggttatcgacggcacatggcagccgaacgatcacgataaggcgaatggtctggtaccgggcttcggagtgaccacgcagatcatcaacggcggcgtggagtgtggcggcccgactgagatcgcccagtctcaaaaccgtatcaaatactacaaagagttcgccaactacctgaaagtgcctgttccgtctaacgaagtgctgggctgcgccaacatgaagcagttcgacgaaggcggtgctggcgcgctgaagatctactgggaacaggactggggatggagcgcggataccccgtcaggccagacctactcttgccagctggtgggttaccagacgccattcagcgcctttaaagagggtgactacaccaaatgcgtgaagcatttcttcaatgtgaacgtggtgggtgaagacgggacttccgacggcggtagcgtcacgccagccccgacgccgacccctgtcgacccaacggacgaaggcaacaccacgccggtgccagacgataacaccccggctccggacgataatacgccagcgccggtaaaccatgcgccggtagcaaaaatcgccggtccggtgggtgcggttgaagcgggtaaatcagtttctctgaacgcgtctggctcgaccgacgaagacggtaaccacctgacttatacctggacggctccgaacggccagaccgtaagcggcgaagataaagcgattattaccttcaatgcgccggaagtggcagcggcgacgcagtacccgatcaaccttaccgtcagtgacggcgagctgagcagcaccaccagctatacgctgaacgtacaggctaagcagaccaacggcggccagaccgggacttacccgacctggacctccaaaaccaaatggaaagcaggcgatatcgttaacaaccgcggccagctgttccagtgcaaaccttatccgtacagcggctggtgcaataacgcgccatcctactacgagccaggtaaagggattgcatggcaggacgcgtggactgcgctgtaa

SEQ ID NO: 7 - DNA, 879 bp, Enterobacter Species
atggacttaacccagcttgaaatgtttaacgccgtcgcgctgacgggcagcatcacccaggcggcgcagaaggtgcatcgcgtgccgtccaacctgacgacccgcatccgccagctggaagccgatcttggcgttgagctgtttattcgtgagaaccagcgtttgcgcttatctcccgccgggcataacttcctgcgctacagcaggcagatcctcgccctggtggatgaagcgcgcatggtcgtcgcgggtgatgagccgcaggggttatttgccctcggcgcgctggaaagcaccgccgcggtgcgcattcccgaaacgctggcgcagtttaaccagcgctatccgcgcattcagtttgccctttctaccgggccttccgggacgatgattgatggcgtactggagggcaccttaagcgccgcctttgtcgacgggccgctgtcgcacccggagctggagggcatgccggtctaccgggaagagatgatgctagtcacgcctgccgggcacgccgaggttgcacgcgccacgcaggttagcggcagcgacgtttacgcatttcgcgcgaactgctcgtaccgtcgacatctggaaagctggtttcatgcggacagagccacgcctggccgcattcatgagatggagtcctaccacggcatgctcgcctgcgtcattgcgggcgcgggcatcgcgctgatgccgcgctcgatgctggagagtatgccgggacatcatcaggttgaagcctggccgctggcggaaaactggcgctggcttacaacctggctggtgtggcggcgcggggcgatgacccgccagctggaagcttttatagcgctgctaaacgaacgtcttcaaccagcgccttctccataa

SEQ ID NO: 8 - DNA, 489 bp, Enterobacter Species
atggtctggattgattacgccatcattgcggtgattggtttttcctgtctggttagcctgatccgtggctttgttcgtgaagcgttatcgctggtgacttggggttgtgctttctttgtcgccagtcattactacacttacctgtctgtctggttcacgggctttgaagatgaactggtccgaaatggaatcgctatcgcggtgctgtttatcgcaacgctgattgtcggcgctatcgtgaattacgtgataggtcagctggtcgagaaaaccggtctgtcaggaacggacagggtactcgggatctgtttcggggcgttgcgaggcgtgctgattgtggccgcgatcctgttcttcctggatacctttaccgggttctccaaaagcgaagactggcagaaatcgcagctcattccagagttcagcttcatcatcagatggttctttgactatctgcaaagctcgtcgagttttttgcccagggcataa

SEQ ID NO: 9 - DNA, 1338 bp, Enterobacter Species
atgacgagccgtaaacctgcccatcttttactggtggatgacgatcccgggctgttaaagctgctggggatgcgtctggtgagtgaaggctacagcgtcgtgaccgccgaaagcgggctggaggggctgaagatcctcacccgcgagaaaatcgatctggtgataagcgacctgcggatggacgaaatggatggcctgcagctgttcgcggagatccagaggcagcagccgggtatgcccgtgatcattctgacggcgcacgggtcgatcccggatgcggttgccgcgacgcagcagggggtcttcagcttcctgaccaagccggtggacaaagacgcgctgtataaggctatcgacagcgcgctggaacatgccgccccggcgggggatgaagcgtggcgggagtccatcgtcacccgcagccccattatgctgcgtctccttgaacaggcccggatggtggcgcagtccgacgtcagcgtgctcatcaacggccagagcggaaccgggaaagagatcctggcccaggcgatccataacgccagcccgcgcagtaaaaatgcctttatcgccattaactgcggcgcgcttccggaacagcttctcgaatctgaactgtttggtcatgcccgcggcgcattcaccggcgcggtgagcagtcgggaagggctgttccaggcggcggaaggcggcacgctgttcctggacgagattggcgacatgcccgcgccgctgcaggtcaaactgctgcgcgtattgcaggagcgaaaagttcgcccgctgggcagcaaccgcgatatcgatattaacgtgcgcattatttccgccacccaccgcgacttgccaaaagtgatggcccgcaacgagtttcgcgaagatctctactaccgtctgaacgtggtgaatctgaagatccctgcgctggcggagcgcgcggaagacattccgctgctggcgaatcatcttctgcgccaggcggccgatcgtcataaaccgtttgtgcgcgcgttttccaccgacgcgatgaagcggctgatggccgcaggctggccgggtaacgtgcgccagctggtgaacgtgattgagcagtgcgtggcgctgacctcctcaccggtaatcagcgatgcgcttgtagagcaggcgctggaaggagaaaacacggcgctgccgacgtttgcggaagcgcggaatcagttcgagctgaactatctgcgcaagctattgcagatcaccaaaggcaacgtgacccacgcggcgcgcatggccggacgcaaccgcaccgagttctacaagctgctgtcgcgccacgagctggaagcaaacgattttaaagagtaa

SEQ ID NO: 10 - DNA, 1464 bp, Enterobacter Species
atgacccacaacatttctctcagaaatagggtgtgcgatgaacaaaagtttgtcatctcagcctgcggttttacgttccagggatatcgtctgttccttaaccagtacggaatacaggcttcgcatattcattttgatggggatgaggcatcgcaacaggatatgaaaaatatccttataaatcagaatgctcatgttgtggtgtttctcggaaaaggtatcttaagtctcctggagagcctgaagcgactggcgtccgtgctcaatgcgttgcccgttattcgacgcgtcacgctgtatggcgacataccggatggctggctatatcgcaccctgggcagtcttttaaataatagttatcaattatcattgattcgactagcccgcgtttcggatgtcgtcactggttctcatacgcaccatcatgtatttaaggaacgttcgtacttattacgcgatcgctacagggataattcttcgcaagacaacgtcaagtggctaacaaaaagagagattgacgttttattaaatttctaccgcggcatgtccgtaaaagaaatgtgcgatgaaatgggactatctaataaaacggtttatacccaccgtaaggaaggcgtgctgaaattacgcttaattaagcggtggctacacgattcgcacaatatcaatgcggaaagaagtatcaagcggcggagtcaaaacacggaatttacggataaggaagcagagatttttaatgcattatataaaaaagagatcttccctgcttatcagatcattaccgatcgtgacaaaaaaggcgtaggctttgagatactgatccgctggaacaaaaacggtaaaattgtcaagccgaccagttttctcacggatatttcaaatcatgagatatggttgaaaattaccgcgctagttattcatgccgccgtgtcgggaattaataagtataatggtaaatactatttttctgtgaatattccacctcgcctggcctccggaaatgcattgcctgatatggcaaggaaagctatcgacatgctgctcaaaccgcagtgggccgagaagctggtctttgaattcgcagaagatattgacgtgacgaaggacaaagggatcccagaaaccatgcggcatttgcgtaatacagggtgtcgattgtttctggatgactgcttctctaatcaccaaaccatgttcccggtgcggcaggtgcattttgatggactcaagctggatcgggatatcgttgagcattttgtggcaaacgacaatgactataacctgatcaaagcgatacagatttatagcgacatgaccggaacggactgtattgcagagggagtagacagtgaggaaaaatttgaaaaattagtcgcgctgggcgtcaaaaactttcagggatattatttatcgcgagccgtgaaagaggacgagttggatcgcatggtcagaatttttagttaa

SEQ ID NO: 11 - DNA, 1563 bp, Enterobacter Species
atgaagcctgaagagttccgcgctgatgccaaacgcccgttaaccggcgaagagtatttaaaaagcctgcaggacggtcgtgagatttatatctacggcgagcgcgtcaaagacgtcaccacccatccggcatttcgcaacgcggcggcctccatcgcgcagatgtacgacgcgctgcacaagcctgacatgcaggacgcgctctgctggggcaccgacaccggcagcggcggctatacccacaagtttttccgcgtggcgaaaagtgccgacgacctgcgccagcagcgcgacgccatcgccgagtggtcgcgcctgagctacggctggatgggccgcacgccggactacaaagccgcgttcggctgcgcgctcggcgccaacccggccttctacggccagttcgaacagaacgcccgtaactggtacacgcgcattcaggaaaccggcctgtactttaaccacgccatcgttaacccgccgatcgaccgtcacaagccggcggacgaggtgaaagacgtctacatcaagctggagaaagagaccgacgccgggattatcgtcagcggcgcgaaagtggtagccaccaactccgccctgacccactacaacatgatcggcttcggctccgcgcaggtgatgggcgaaaacccggacttcgcgctgatgttcgtcgcgccgatggatgccgaaggcgtgaagctgatctcccgcgcctcttacgaaatggtggcaggcgcaaccggatccccgtacgactacccgctctccagccgctttgatgagaacgacgcgatcctggtgatggatcacgtgctgatcccgtgggaaaatgtgctgatctaccgcgatttcgaccgctgccgtcgctggacgatggaaggcggtttcgcgcggatgtacccgctgcaggcctgcgtgcgtctggcggtgaagctcgacttcatcaccgccctgctgaagaaatccctggagtgcaccggcaccctggagttccgcggcgtacaggcggatctcggcgaagtggtggcctggcgtaacatgttctgggcgctgagcgactccatgtgttccgaagccaccccgtgggtcaacggcgcgtatctgccggatcacgccgcgctgcaaacctaccgcgtgatggcgccgatggcctacgccaagatcaagaacatcatcgagcgtaacgtgacgtccggtctgatctacctgccgtccagcgcccgggatctgaacaacccgcagatcgaccagtatctggcgaaatacgtgcgcggctccaacgggatggatcacgtcgaacgcatcaagattttgaagctgatgtgggatgccatcggcagcgagtttggcggtcgtcacgagctatacgaaatcaactactccggcagccaggatgagatccgcctgcagtgtctgcgccaggcgcagagctccggcaacatggacaaaatgatggcgatggtcgaccgctgcatgtccgaatacgaccagcacggctggaccgtaccgcacctgcacaacaacagcgatatcaacatgctcgacaagctgctgaaatag

SEQ ID NO: 12 - protein, 284 aa, Enterobacter Species
VNTIGINSEPILTHSGFSITADTTLAADRHYDVIYLPALWRNPRAVVRQQPELLAWLSEQAARGTRIAAVGTGCCFLAESGLLNGKPATTHWHYFKQFSRDYPNVKLQTKHFLTQADNIYCAASVKALSDLTIHFIETIYGKRVATHTQRTFFHEIRSQFDRQCYSEENKPHPDEDIVQIQIWIKANCASDISMQNLADMAGMSLRNFNRRFKNATDISPLQYLLTARIESAMTMLQSTNLSIQEIANAVGYQDIAHFNRQFKHKTTVSPGDYRKTVRAKMFSA

SEQ ID NO: 13 - protein, 2478 aa, Enterobacter Species
MPAKSSGSAWERFAGVLRNAQTECIVTTAKGAETLGQLSLPLSPLIFTFDKPDTAALPAGYRLHPLDRTFSGAFHPVPVAENDLAFLQYTSGSTGSPKGVMVTHGNLWANSHAIHRFFGHHSESRGTIWLPHFHDMGLIGGLLQPVFGAFPCRVMSPMMLMKNPLNWLKHISDYQATTSGGPNFAYDLCVRKIGREQVEALDLSRWDVAFCGAEPIRPATLQQFSEHFAPAGFRPGAFLPCYGMAETTLIVTGMEKGQGLRVSDEAGTVSCGQALPDTEVRIVDPDRHQPLADGESGEIWLRGPSVAAGYWDNDAATRETFQASLAGHPHPWLRSGDMGFLQSGHLYVTGRLKELLIINGQNHYPTDIEETIRQADPALAEATVCVFASEDERPVALLELMTRHKNDLDMATLAPSVTAAVAERHGITLDELLLVGRRAIPRTTSGKLQRTRAKAMHQQGTLEVAWRSCQDASKPVELAGETPPALAALIAGIISSAMNTTIGESQWDEAFTGFGMSSLQAVGVIGELEQRLGRELSPALIYDYPTINRLAAALGQPAAARPVSSAVAESAIAVIGIGVELPGHSGVEALWSLLQQGHSTTGEIPAHRWRTSSLDGFNRKGSFFDEVDAFDAGYFGISPREAVYIDPQHRLLLETVQQALTDAGLKASSLRGSDTAVFVGISASDYALACGDNVSAYSGLGNAHSIAANRISYLYDLKGPSVAVDTACSSSLVAIEGAMQSLRAGRCALAIAGGVNLALTPHLQKVFTEAQMLAPDGRCKTFDARADGYVRGEGCGVVVLKPLSQALADGDRVYATLVASAVNQDGRSNGITAPNGPSQQAVILQAMADAGLDSDSIDYIEAHGTGTALGDLIEYQALEAVFADRKKTAPVQVGSIKTNIGHLEAAAGVLGVVKTSLMLHFRQYVPHLNFQQKNPHIAAISRHVEVSGAQPASWHADGEARYAGVSSFGFGGTNGHVILRSAPAVEKRQEPAAPHGLLLVGSHDKGAFTLQREAVKKGLSTCQESDIATWCRLVNTRYDAARYRGVAYGADRSQLAESLAQLTVCKVGKAQPQVWLFPGQGTQQIGMGAELYHHLPHYRTQFDALATTIQQRYQIDITQALFARDDSWQRCARTCQLSLFACSYALAQSVMQFGPRPAAVMGHSLGEYCAAVIAGYLSLDDGLAMVHQRALLMSALTQEGAMAVVFSGEADVRQMISPWTGDIDIAAFNTPTLTTIAGSRAAIDACLQAISSKGGHARKIKTASAFHSSMMDPILGAWREWLVNNVTFTRGTIPFYSNLNGEACDRTDADYWTRQIRQPVSFLQGVQNVLAQGEFTFIDLSADGSLGKFVTATDRRHRVLAAGDRRHEYKSLLTLLGTLWQQGHDINWSGLYHATTREALTLPAIQFCRKRYWLAGETPAQTPSAKEDAMSNQHHLAAEIKAIIAGFLEADPAALDDSLPFLEMGADSLVLLDAINTIKDRFGVAIPVRALFEELNTLDAVIGYVVEHAQPAASLTTPETAGLAAQPVAAPQGTSRPVPDTVQDLIARQLELMSQQLNLLNGTAQALPMPAAPATPDVIAPAPVVAPTAPVKASAHNSWFKKETKKVSLGAERDQHLAQLTERFVDKTGGSKRNAQQYRAVLADNRASAGFRLSTKEMLYPLVGERSQGSRIWDVDGNEYIDFTMGFGANLLGHAPDCVQQAVADQLARGMQIGPQSALAGEVATLISELTGQQRVAFCNSGSEAVMSAVRLARAVTGKNKVALFSGSYHGVFDGILGRQQGGETPERATPIAAGTPPSLVDDLLVLDYGSEESLALIARYAAELAVVIVEPVQSRYPDHQPRDYLHTLRELTTTHNIALMFDEVITGFRLAAGGAQAYYGVQADIASYGKIVGGGMPIGVIAGSARFMDSIDGGFWQYGDDSWPQAELIFFAGTFSKHPLTMAASKAVLEYIKTHPALYDDINQKTARLAMSLNTWFSATGTPIEIVSAGSLFRFKFNGNYDILFHHLMLRGIFIWEGRNCFVSVAHADEDIDRFIAAVKESVNAMRVDGFFGEAGLRPDTRYAVADSQQRFLQLAAQDESGLLAGTIGGVIETPRNVDGDIMRAAWQLLCERHDALRMQFTDAGDLQVALAPIVDICEENAAPAACLAEFAARPFDLTATPLARLLLVRHEGKTTLAISAHHTVADGWSFMVMLRELLHLYDALASGKRPDLAAAASYLQAIRAQNIVSEAELPARLAALPARRVTPEVLSVQDPSLTYQGQRLVQRLSYPGLTGQLRKASAELRVTRFAMLNALFTLTLEKAFGHSPVPVGVPDAGRDFGQGDALVGQCVRLLPLCIDSAACASVSGVARAIHDGILAQRGEPALPSRCFHGQDAPLPLLATFNVEPHAPLAEMRQWEASLSLLPIGAVEFPLMVNILETKEGLSVELDYQMRYFTEERARALLEHFLKAITVLAEQGEEAVEALFSSSEALAAS

SEQ ID NO: 14 - protein, 403 aa, Enterobacter Species
MSWQYFKQTYLVKFWSPVPAVIAAGILSTYYFGITGTFWAVTGEFTRWGGQLLQLAGVHAEEWGYFKLIHLDGTPLTRIDGMMIVGMFGGCFAAALWANNVKLRMPKSRIRIMQAVGGGIIAGFGARLAMGCNLAAFFTGIPQFSLHAWFFAVATAIGSYFGAKFTLLPLFRIPVKMTKVSAASPLTQKPDQARRRFRLGMLVFLAMVAWALCTAMNQPKLGLAMLFGVGFGLLIERAQICFTSAFRDMWITGRTMMAKAIIAGMAVSAIGIFSYVQLGVEPKIMWAGPNAVIGGLLFGFGIVLAGGCETGWMYRAVEGQVHYWWVGLGNVIGSTILAYFWDDLSPALATSWDKVNLLSTFGPLGGLLVTYALLLVAFLLVVAQEKRFFRRASVKTETQENAA

SEQ ID NO: 15 - protein, 698 aa, Enterobacter Species
MKRTYLYSMLALCVSAACHAETYPAPIGPSQSDFGGVGLLQTPTARMAREGEISLNYRDNDQYRYYSASVQLFPWLETTLRYTDVRTKQYSSVDAFSGDQTYKDKAFDVKLRLWEESYWMPQVSVGAKDIGGTGLFDAEYIVASKAWGPFDFSLGLGWGYLGTGGNVKNPFCSYSDKYCYRDNSYKKAGSINGDQMFHGPASLFGGVEYQTPWQPLRLKLEYEGNDYSQDFAGKIEQKSKFNVGAIYRVTDWADVNLSYERGNTVMFGFTLRTNFNDMRPHYNDNARPAYQPEPQDAILQHSVVANQLTLLKYNAGLADPKIQVKGDTLYVTGEQVKYRDSREGIERANRIVMNDLPEGIRTIRVTENRLNLPQVTTETDVASLKRHLEGEPLGHETELVQKRVEPIVPETTEQGWYIDKSRFDFHIDPVLNQSVGGPENFYMYQLGVMATADLWLTDHLLTTGSLFGNIANNYDKFNYTNPPKDSQLPRVRTRVREYVQNDAYVNNLQANYFQYFGNGFYGQVYGGYLETMYGGAGAEVLYRPVDSNWAFGVDANYVKQRDWRSAQDMMKFTDYSVKTGHLTAYWTPSFAPDVLVKASVGQYLAGDKGGTLDISKHFDSGVVVGGYATITNVSPDEYGEGDFTKGVYVSIPLDLFSSGPTRSRAAVGWTPLTRDGGQQLGRKFQLYDMTSDKNINFR

SEQ ID NO: 16 - protein, 609 aa, Enterobacter Species
MNKRTLLSVLIAGACVAPFMAQATLLQASSEPYTLKASDLQKKEQELTNFPLMASVKSTIRTLDNSLVEQIEPGKSTNPENVKRVEGIIKASDWDYLFPLRAPEYTYSNFLKAVGKFPALCQTYTDGRNSDAICRKSLATMFAHFAQETGGHESWRPEAEWRQALVYVREMGWSEGQKGGYNGECNPDVWQGQTWPCGKDKDGDFVSYFGRGAKQLSYNYNYGPFSEAMYGDVRVLLDKPELVADTWLNLASAIFFFAYPQPPKPSMLQVIDGTWQPNDHDKANGLVPGFGVTTQIINGGVECGGPTEIAQSQNRIKYYKEFANYLKVPVPSNEVLGCANMKQFDEGGAGALKIYWEQDWGWSADTPSGQTYSCQLVGYQTPFSAFKEGDYTKCVKHFFNVNVVGEDGTSDGGSVTPAPTPTPVDPTDEGNTTPVPDDNTPAPDDNTPAPVNHAPVAKIAGPVGAVEAGKSVSLNASGSTDEDGNHLTYTWTAPNGQTVSGEDKAIITFNAPEVAAATQYPINLTVSDGELSSTTSYTLNVQAKQTNGGQTGTYPTWTSKTKWKAGDIVNNRGQLFQCKPYPYSGWCNNAPSYYEPGKGIAWQDAWTAL

SEQ ID NO: 17 - protein, 292 aa, Enterobacter Species
MDLTQLEMFNAVALTGSITQAAQKVHRVPSNLTTRIRQLEADLGVELFIRENQRLRLSPAGHNFLRYSRQILALVDEARMVVAGDEPQGLFALGALESTAAVRIPETLAQFNQRYPRIQFALSTGPSGTMIDGVLEGTLSAAFVDGPLSHPELEGMPVYREEMMLVTPAGHAEVARATQVSGSDVYAFRANCSYRRHLESWFHADRATPGRIHEMESYHGMLACVIAGAGIALMPRSMLESMPGHHQVEAWPLAENWRWLTTWLVWRRGAMTRQLEAFIALLNERLQPAPSP

SEQ ID NO: 18 - protein, 162 aa, Enterobacter Species
MVWIDYAIIAVIGFSCLVSLIRGFVREALSLVTWGCAFFVASHYYTYLSVWFTGFEDELVRNGIAIAVLFIATLIVGAIVNYVIGQLVEKTGLSGTDRVLGICFGALRGVLIVAAILFFLDTFTGFSKSEDWQKSQLIPEFSFIIRWFFDYLQSSSSFLPRA

SEQ ID NO: 19 - protein, 445 aa, Enterobacter Species
MTSRKPAHLLLVDDDPGLLKLLGMRLVSEGYSVVTAESGLEGLKILTREKIDLVISDLRMDEMDGLQLFAEIQRQQPGMPVIILTAHGSIPDAVAATQQGVFSFLTKPVDKDALYKAIDSALEHAAPAGDEAWRESIVTRSPIMLRLLEQARMVAQSDVSVLINGQSGTGKEILAQAIHNASPRSKNAFIAINCGALPEQLLESELFGHARGAFTGAVSSREGLFQAAEGGTLFLDEIGDMPAPLQVKLLRVLQERKVRPLGSNRDIDINVRIISATHRDLPKVMARNEFREDLYYRLNVVNLKIPALAERAEDIPLLANHLLRQAADRHKPFVRAFSTDAMKRLMAAGWPGNVRQLVNVIEQCVALTSSPVISDALVEQALEGENTALPTFAEARNQFELNYLRKLLQITKGNVTHAARMAGRNRTEFYKLLSRHELEANDFKE

SEQ ID NO: 20 - protein, 487 aa, Enterobacter Species
MTHNISLRNRVCDEQKFVISACGFTFQGYRLFLNQYGIQASHIHFDGDEASQQDMKNILINQNAHVVVFLGKGILSLLESLKRLASVLNALPVIRRVTLYGDIPDGWLYRTLGSLLNNSYQLSLIRLARVSDVVTGSHTHHHVFKERSYLLRDRYRDNSSQDNVKWLTKREIDVLLNFYRGMSVKEMCDEMGLSNKTVYTHRKEGVLKLRLIKRWLHDSHNINAERSIKRRSQNTEFTDKEAEIFNALYKKEIFPAYQIITDRDKKGVGFEILIRWNKNGKIVKPTSFLTDISNHEIWLKITALVIHAAVSGINKYNGKYYFSVNIPPRLASGNALPDMARKAIDMLLKPQWAEKLVFEFAEDIDVTKDKGIPETMRHLRNTGCRLFLDDCFSNHQTMFPVRQVHFDGLKLDRDIVEHFVANDNDYNLIKAIQIYSDMTGTDCIAEGVDSEEKFEKLVALGVKNFQGYYLSRAVKEDELDRMVRIFS

SEQ ID NO: 21 - protein, 520 aa, Enterobacter Species
MKPEEFRADAKRPLTGEEYLKSLQDGREIYIYGERVKDVTTHPAFRNAAASIAQMYDALHKPDMQDALCWGTDTGSGGYTHKFFRVAKSADDLRQQRDAIAEWSRLSYGWMGRTPDYKAAFGCALGANPAFYGQFEQNARNWYTRIQETGLYFNHAIVNPPIDRHKPADEVKDVYIKLEKETDAGIIVSGAKVVATNSALTHYNMIGFGSAQVMGENPDFALMFVAPMDAEGVKLISRASYEMVAGATGSPYDYPLSSRFDENDAILVMDHVLIPWENVLIYRDFDRCRRWTMEGGFARMYPLQACVRLAVKLDFITALLKKSLECTGTLEFRGVQADLGEVVAWRNMFWALSDSMCSEATPWVNGAYLPDHAALQTYRVMAPMAYAKIKNIIERNVTSGLIYLPSSARDLNNPQIDQYLAKYVRGSNGMDHVERIKILKLMWDAIGSEFGGRHELYEINYSGSQDEIRLQCLRQAQSSGNMDKMMAMVDRCMSEYDQHGWTVPHLHNNSDINMLDKLLK

SEQ ID NO: 22 - DNA, 19 bp, Artificial Sequence
  Synthetic
ggttaccttgttacgactt

SEQ ID NO: 23 - DNA, 19 bp, Artificial Sequence
  Syntheric
  misc_feature (4)..(4): m = a or c
  misc_feature (22)..(22): l = g or t
  misc_feature (22)..(22): k = g or t
aacmggattagataccckg

SEQ ID NO: 24 - DNA, 22 bp, Artificial Sequence
  Synthetic
accccggtcatctctgataatg

SEQ ID NO: 25 - DNA, 19 bp, Artificial Sequence
  Synthetic
gcgcgctgtctctttcatc

SEQ ID NO: 26 - DNA, 19 bp, Artificial Sequence
  Synthetic
ggcgcgcgttatcattgta

SEQ ID NO: 27 - DNA, 18 bp, Artificial Sequence
  Synthetic
cgctgcgcaccaacttta

SEQ ID NO: 28 - DNA, 20 bp, Artificial Sequence
  Synthetic
gtgctgaagcgatcttaggg

SEQ ID NO: 29 - DNA, 20 bp, Artificial Sequence
  Synthetic
gttccatcaggctttttcca

SEQ ID NO: 30 - DNA, 20 bp, Artificial Sequence
  Synthetic
ggcataacttcctgcgctac

SEQ ID NO: 31 - DNA, 20 bp, Artificial Sequence
  Synthetic
cagtacgccatcaatcatcg

SEQ ID NO: 32 - DNA, 18 bp, Artificial Sequence
  Synthetic
cgcttcgcgatgctgaat

SEQ ID NO: 33 - DNA, 17 bp, Artificial Sequence
  Synthetic
ggccgaacgccttctca

SEQ ID NO: 34 - DNA, 24 bp, Artificial Sequence
  Synthetic
tgttttatgcttaaactggcgatt

SEQ ID NO: 35 - DNA, 19 bp, Artificial Sequence
  Synthetic
cgaatgcggtgggatatca

SEQ ID NO: 36 - DNA, 18 bp, Artificial Sequence
  Synthetic
gacgatgcgaccggtttt

SEQ ID NO: 37 - DNA, 19 bp, Artificial Sequence
  Synthetic
agcctatcgaccggatgct

SEQ ID NO: 38 - DNA, 17 bp, Artificial Sequence
  Synthetic
tggcgggagtccatcgt

SEQ ID NO: 39 - DNA, 20 bp, Artificial Sequence
  Synthetic
tgttcaaggagacgcagcat

SEQ ID NO: 40 - DNA, 19 bp, Artificial Sequence
  Synthetic
ccgacctggacctccaaaa

SEQ ID NO: 41 - DNA, 19 bp, Artificial Sequence
  Synthetic
gccgcggttgttaacgata

SEQ ID NO: 42 - DNA, 20 bp, Artificial Sequence
  Synthetic
ggggacagtaacgacgaaac

SEQ ID NO: 43 - DNA, 20 bp, Artificial Sequence
  Synthetic
cggcaatctgtcgatatgaa

SEQ ID NO: 44 - DNA, 23 bp, Artificial Sequence
  Synthetic
ggagtcaaaacacggaatttacg

SEQ ID NO: 45 - DNA, 26 bp, Artificial Sequence
  Synthetic
atctgataagcagggaagatctcttt

SEQ ID NO: 46 - DNA, 22 bp, Artificial Sequence
  Synthetic
gcagtttgacgcccttaatgtt

SEQ ID NO: 47 - DNA, 17 bp, Artificial Sequence
  Synthetic
ggcgcaggagagtgcaa

SEQ ID NO: 48 - DNA, 20 bp, Artificial Sequence
  Synthetic
tcaaacgttcagcattgagc

SEQ ID NO: 49 - DNA, 20 bp, Artificial Sequence
  Synthetic
tatgccggattaacgacctc

SEQ ID NO: 50 - DNA, 20 bp, Artificial Sequence
  Synthetic
ggggacagtaacgacgaaac

SEQ ID NO: 51 - DNA, 20 bp, Artificial Sequence
  Synthetic
cggcaatctgtcgatatgaa

SEQ ID NO: 52 - DNA, 20 bp, Artificial Sequence
  Synethic
tacgttgaagcccgtaaagg

SEQ ID NO: 53 - DNA, 20 bp, Artificial Sequence
  Synthetic
agaaaaagcggctgacaaaa